SEQUENCE LISTING (2 sequences)

SEQ ID NO: 1 - protein, 71 aa, synthetic construct
MGDRRKEMDKVYRTAYKRITSTPDKEKRKEVVKEATEQLRRIAKDEEEKKKAAYMISFLKTLGLEHHHHHH

SEQ ID NO: 2 - protein, 21 aa, synthetic construct
MSHHHHHHHHSENLYFQSGGG

(19) United States
(12) Patent Application Publication
(10) Pub. No.: US 2026/0265696 A1
(43) Pub. Date: Sep. 10, 2026

(54) HUMAN IPSC DERIVED AMELOBLASTS AND USES THEREOF

(71) Applicant: UNIVERSITY OF WASHINGTON, Seattle, WA (US)

(72) Inventors: Hannele RUOHOLA-BAKER, Seattle, WA (US); Ammar ALGHADEER, Seattle, WA (US); Hai ZHANG, Seattle, WA (US); Julie MATHIEU, Seattle, WA (US); Sesha HANSON-DRURY, Seattle, WA (US); Yan Ting ZHAO, Seattle, WA (US); Devon EHNES, Seattle, WA (US); Yuliang WANG, Seattle, WA (US); David BAKER, Seattle, WA (US)

(73) Assignee: UNIVERSITY OF WASHINGTON, Seattle, WA (US)

(21) Appl. No.: 18/723,167

(22) PCT Filed: Dec. 20, 2022
(86) PCT No.: PCT/US2022/053517

§ 371 Date: Jun. 21, 2024

Related U.S. Application Data
(60) Provisional application No. 63/294,165, filed on Dec. 28, 2021.

(51) Int. Cl. C12N 5/077; A61K 35/32; A61K 38/17

(52) U.S. Cl. CPC C12N 5/0654; A61K 35/32; A61K 38/1709; C12N 2501/11; C12N 2501/13; C12N 2501/15; C12N 2501/155; C12N 2501/41; C12N 2501/415; C12N 2506/45

(57) ABSTRACT

Enamel forms during early development, before the tooth breaks through the gum, and it covers the visible portion of the tooth. During development, epithelial cells within the enamel organ differentiate into ameloblasts, which produce the hardened, high mineral-content enamel. The technology as described herein relates to discovery of methods for generating ameloblasts or organoids thereof in vitro and compositions that utilize the generated ameloblasts for restoring enamel products.

### CROSS REFERENCE TO RELATED APPLICATIONS

The application claims benefit under 35 U.S.C. § 119(e) of U.S. Provisional Application No. 63/294,165, filed Dec. 28, 2021, the contents of which are incorporated herein by reference in its entirety.

### GOVERNMENT SUPPORT PARAGRAPH

This invention was made with government support under Grant Nos. U01HL099997 and UO1HL099993, awarded by the National Heart, Lung, and Blood Institute and Grant Nos. 1P01GM081619 and R01GM083867 and R01GM097372 and R01GM97372-0351, awarded by the National Institutes of Health. The government has certain rights in the invention.

### TECHNICAL FIELD

The technology described herein relates to the generation of ameloblasts and uses thereof.

### BACKGROUND

There is much interest in the dental field for methods of maintaining or repairing damage to enamel caused by demineralization of teeth due to dental plaques. The cells that produce enamel (e.g., ameloblasts) are not present in adults, thus reversing enamel loss is not always possible.

Enamel forms during early development, before the tooth breaks through the gum, and it covers the visible portion of the tooth. During development, epithelial cells within the enamel organ differentiate into ameloblasts, which produce the hardened, high mineral-content enamel. More specifically, the enamel organ appears naturally in vivo as an aggregate of cells in histologic sections of the developing tooth, and it includes the inner enamel epithelium, where ameloblasts reside, the outer enamel epithelium, the stratum intermedium, and the stellate reticulum. The dental organ is comprised of the enamel organ and mesenchyme.

### SUMMARY

The methods and compositions described herein are based, in part, on the discovery of methods for generating ameloblasts or organoids thereof in vitro from induced pluripotent stem cells (iPSCs). Also provided herein are compositions comprising in vitro-derived ameloblasts for administration or transplantation into a subject to induce enamel production to treat enamel loss or demineralization. Also provided herein, are enamel products produced using the ameloblast or ameloblast organoids produced as described herein.

Accordingly, provided herein in one aspect is a method of preparing an ameloblast culture, the method comprising, in order, a) contacting, in culture, an induced pluripotent stem cell (iPSC) with an activator of the Hedgehog pathway; b) adding Bone Morphogenetic Protein 4 (BMP4) to the culture of (a); c) adding an inhibitor of BMP type I receptors, a Wnt activator, Epidermal Growth Factor (EGF) and Neurotrophin-4 (NT4) to the culture of (b) and incubating to form oral epithelium cells; and d) adding BMP4 and transforming growth factor β1 (TGF-β1) to the culture of (c) and incubating to form ameloblasts.

In one embodiment of this aspect and all other aspects described herein, the ameloblasts express ameloblastin.

In another embodiment of this aspect and all other aspects described herein, the cells are human.

In another embodiment of this aspect and all other aspects described herein the iPSCs are seeded on tissue culture plates coated with an extracellular matrix composition.

In another embodiment of this aspect and all other aspects described herein, the extracellular matrix composition comprises a natural or a synthetic extracellular matrix composition.

In another embodiment of this aspect and all other aspects described herein, the iPSCs are grown to confluence prior to step (a).

In another embodiment of this aspect and all other aspects described herein, the iPSCs are cultured to confluence in mTeSR1 stem cell medium.

In another embodiment of this aspect and all other aspects described herein, the Hedgehog activator is smoothened agonist (SAG).

In another embodiment of this aspect and all other aspects described herein, confluent iPS cells are switched to medium comprising the Hedgehog activator at day zero of differentiation.

In another embodiment of this aspect and all other aspects described herein, SAG is added at 200 nM to 1 μM.

In another embodiment of this aspect and all other aspects described herein, SAG is added at 400 nM.

In another embodiment of this aspect and all other aspects described herein, addition of BMP4 step (b) is performed at day 3 of differentiation.

In another embodiment of this aspect and all other aspects described herein, addition of BMP4 step (b) adds BMP4 at 100 μM to 750 μM.

In another embodiment of this aspect and all other aspects described herein, addition of BMP4 step (b) adds BMP4 at 150 μM.

In another embodiment of this aspect and all other aspects described herein, cells are incubated with BMP4 of step (b) from day 3 until day 7 of differentiation.

In another embodiment of this aspect and all other aspects described herein, step (c) addition of an inhibitor of BMP type I receptors, a Wnt activator, EGF and NT4 is performed at day 8 of differentiation.

In another embodiment of this aspect and all other aspects described herein, the inhibitor of BMP type I receptors is LDN-193189.

In another embodiment of this aspect and all other aspects described herein, the LDN-193189 is added at 100 nM to 5 μM.

In another embodiment of this aspect and all other aspects described herein, LDN-193189 is added at 1 μM.

In another embodiment of this aspect and all other aspects described herein, the Wnt activator is a GSK-3 inhibitor.

In another embodiment of this aspect and all other aspects described herein, the GSK-3 inhibitor is CHIR99021.

In another embodiment of this aspect and all other aspects described herein, CHIR99021 is added at 0.5 μM to 50 μM.

In another embodiment of this aspect and all other aspects described herein, the CHIR99021 is added at 5 μM.

In another embodiment of this aspect and all other aspects described herein, EGF is added at 50 μM to 5 nM.

In another embodiment of this aspect and all other aspects described herein, EGF is added at 500 μM.

In another embodiment of this aspect and all other aspects described herein, NT4 is added at 350 nM to 35 μM.

In another embodiment of this aspect and all other aspects described herein, NT4 is added at 3.5 μM.

In another embodiment of this aspect and all other aspects described herein, step (d) addition of BMP4 and TGF-β1 is performed at day 10 of differentiation.

In another embodiment of this aspect and all other aspects described herein, step (d) addition of BMP4 and TGF-β1 is performed when expression of one or more of PITX2, TBX1 and TP63 is detected in the differentiating culture.

In another embodiment of this aspect and all other aspects described herein, the BMP-4 added at step (d) is added at 30 pM to 3 nM.

In another embodiment of this aspect and all other aspects described herein, the BMP-4 added at step (d) is added at 300 pM.

In another embodiment of this aspect and all other aspects described herein, the TGF-β1 is added at 80 nM to 8 μM.

In another embodiment of this aspect and all other aspects described herein, the TGF-β1 is added at 800 nM.

In another embodiment of this aspect and all other aspects described herein, the incubating of step (d) is to day 16 or more of differentiation.

Another aspect provided herein relates to a cultured organoid comprising in an in-vitro-differentiated ameloblast.

In one embodiment of this aspect and all other aspects described herein, the ameloblast is differentiated from an iPS cell.

In another embodiment of this aspect and all other aspects described herein, the ameloblast is human.

In another embodiment of this aspect and all other aspects described herein, ameloblastin is secreted into a lumen in the organoid.

Also provided herein in another aspect is a tooth comprising a dental repair composition comprising ameloblastin produced by an in vitro-differentiated cell.

Another aspect provided herein relates to a tooth comprising a dental repair composition comprising enamel produced by an in vitro-differentiated cell.

In one embodiment of this aspect and all other aspects described herein, the dental repair composition further comprises calcium phosphate or hydroxyapatite.

In another embodiment of this aspect and all other aspects described herein, the dental repair composition further comprises one or more of amelogenin and enamelin.

Also provided herein, in another aspect, is a dental repair composition comprising ameloblastin produced by an in vitro-differentiated cell.

Another aspect provided herein relates to a dental repair composition comprising enamel produced by an in vitro-differentiated cell.

In one embodiment of this aspect and all other aspects described herein, the dental repair composition comprises hydroxyapatite or calcium phosphate.

In another embodiment of this aspect and all other aspects described herein, the repair dental composition further comprises one or more of amelogenin and enamelin.

In another embodiment of this aspect and all other aspects described herein, the in vitro-differentiated cell is an ameloblast differentiated from an iPS cell.

### BRIEF DESCRIPTION OF DRAWINGS

FIGS. 1A-1F: Human tooth and salivary gland exhibit stepwise developmental processes (FIG. 1A, ID). The oral epithelium will give rise to the epithelial components of teeth and salivary glands, while the condensed dental ectomesenchyme (DEM, colored in grey) will give rise to the mesenchymal component of these tissues. TG: tooth germ, DF: dental follicle, DP: dental papilla, P-de: pre-dentin, De: dentin, En: enamel matrix. (FIG. 1B, 1E) Human fetal tooth germs and salivary glands were dissected in a stage-specific manner from human fetal jaw tissue. The young fetal jaws, 9-11 gestational weeks were segmented into anterior segments (dotted box, which span from canine-to-canine region), and posterior jaw segment pairs (dotted box) and sequenced independently. For older fetal jaws, 12-22 gestational weeks, individual tooth germs and submandibular salivary glands were dissected. A more detailed look at the dissection process can be found in FIG. 7B and FIG. 9B. (FIG. 1C, IF)

FIGS. 1G-1H: Density plots of the clustered sci-RNA-seq data highlight the location of each tissue type in the same UMAP coordinate in FIG. 1G. The UMAP graph (FIG. 1G) yielded 20 annotated clusters from all sequenced data. (FIG. 1H) Immunofluorescence staining of developing toothgerms tissue sections with anti-Krt5 that specifically marks the dental epithelial morphology throughout the developmental stages. Counterstained with the nuclear staining DAPI. Abbreviations: incisal edge (IE), cervical loop (CL).

FIGS. 1I-1N: To establish expression of known odontoblast and ameloblast markers in the tissue, immunofluorescence was performed on human fetal toothgerm at 20 gw using dentin sialophosphoprotein (DSPP) and ameloblastin (AMBN), respectively (FIGS. 1J-1K) higher magnification in (FIGS. 1M-1N). Simplified illustration in (FIGS. 1I, IL). As expected, ameloblasts marked by AMBN (FIGS. 1J, 1M), and odontoblasts by DSPP (FIGS. 1K, IN). Scale bars: 50 μm.

FIGS. 2A-2B: (FIG. 2A) UMAP graph of subclustered molar and incisor tooth germ type dental mesenchyme derived cells from the total dataset identified 5 transcriptionally unique clusters including dental ectomesenchyme (DEM), preodontoblast (POB), odontoblast (OB), subodontoblast progenitor (SOBP) and subodontoblast (SOB). (FIG. 2B) A custom heatmap was generated to identify the marker genes specific to each cluster, the top associated GO-terms to characterize cluster function, and calculated Age Score per cluster.

FIGS. 2C-2H: Comparison of the incisor and molar tooth germ types timepoint at which OB first appear (FIG. 2C), proportions of cell type populations (FIG. 2D), and developmental scores calculated from expression profile of DEM and OB markers to determine differentiation state (FIG. 2E). (FIG. 2F) UMAP graph of subclustered incisor only dental mesenchyme derived cells identified the same 5 transcriptionally unique clusters. (FIG. 2G) Pseudotime trajectory analysis for incisor dental mesenchyme derived cells indicates two lineages giving rise to OB. (FIG. 2H) Simplified OB differentiation trajectory tree illustrating main OB lineage, of DEM giving rise to POB, which then gives rise to OB; and SOB lineage, of SOBP giving rise to SOB, which transitions through a POB-like state before giving rise to OB.

FIG. 21 shows a diagram of the developing dental mesenchyme derived cell types of the incisor tooth germ. At 9-11 gestational weeks only the DEM are present. By 12-13 gestational weeks, the population is mixed with SOBP, which is maintained until 14-16 gestational weeks. By 17-19 gestational weeks, both progenitor sources are no longer present and have given rise to POB and SOB. Finally, at 20-22 gestational weeks, the dental mesenchyme tissue mostly consists of OB, POB and SOB.

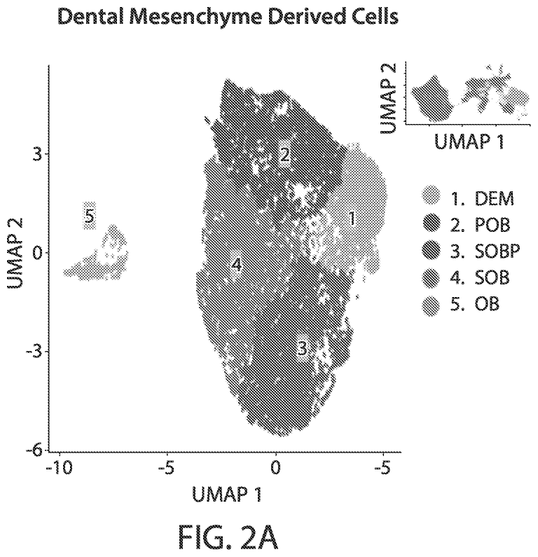

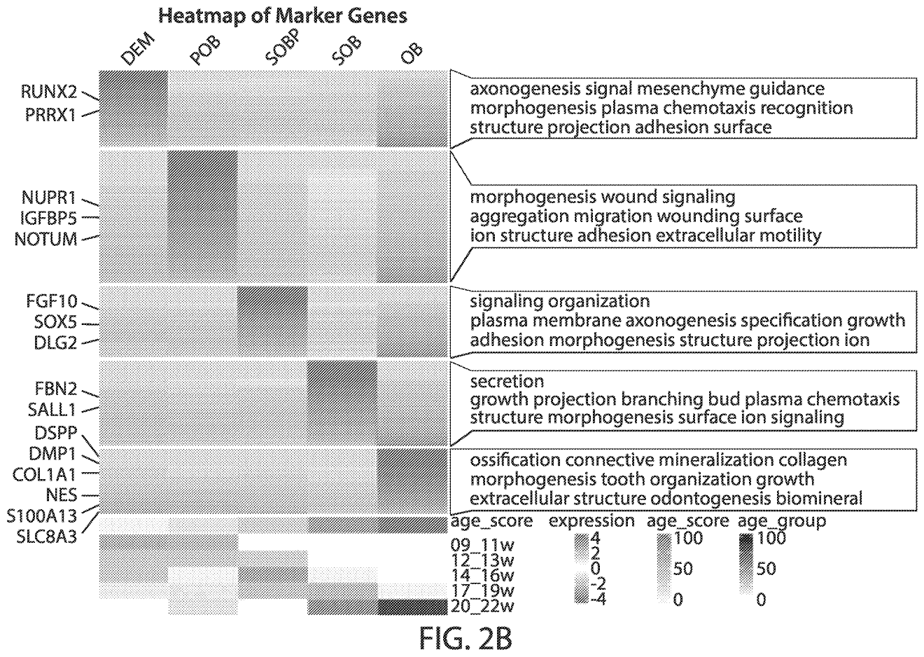

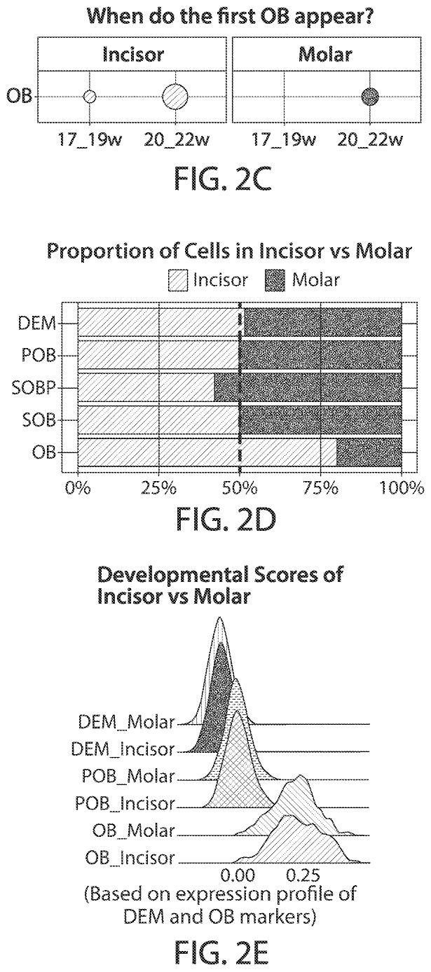

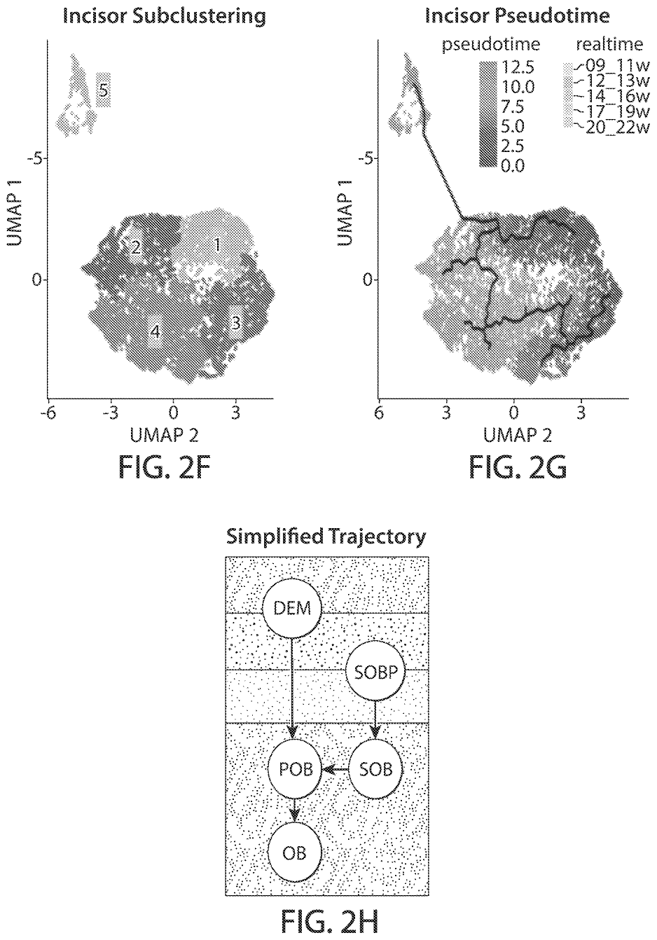

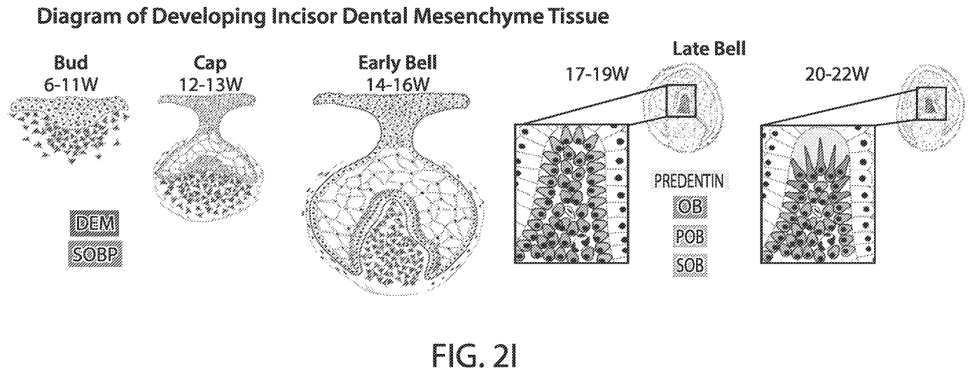

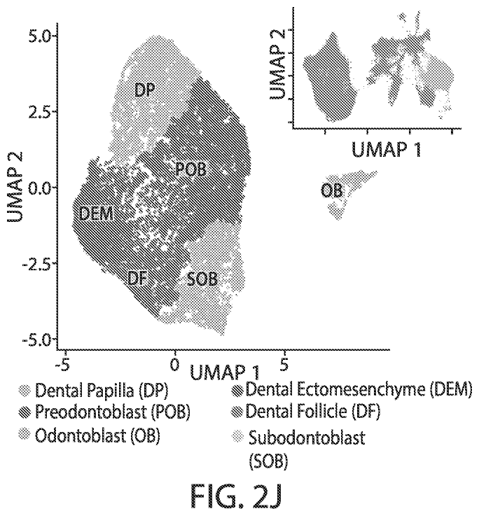

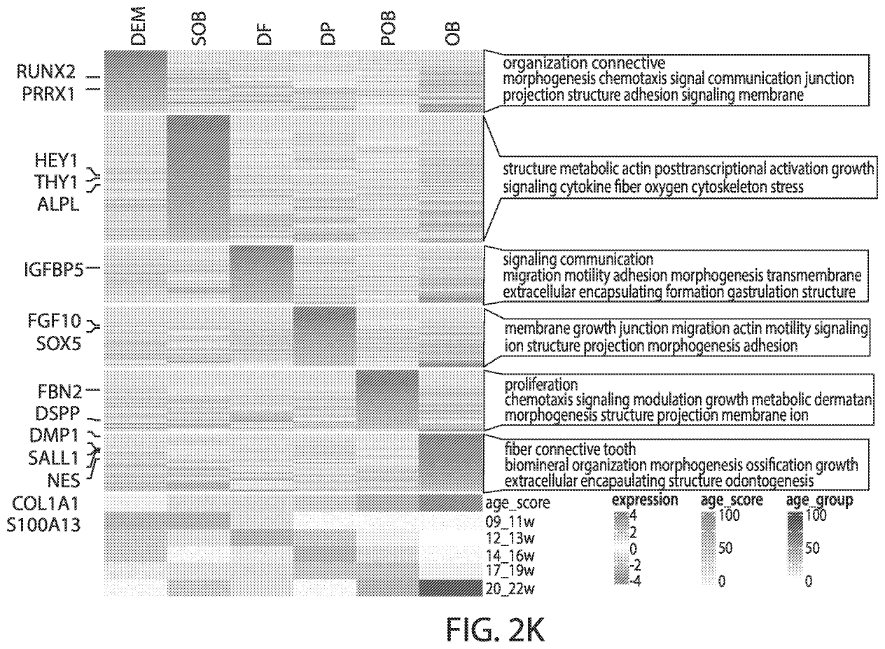

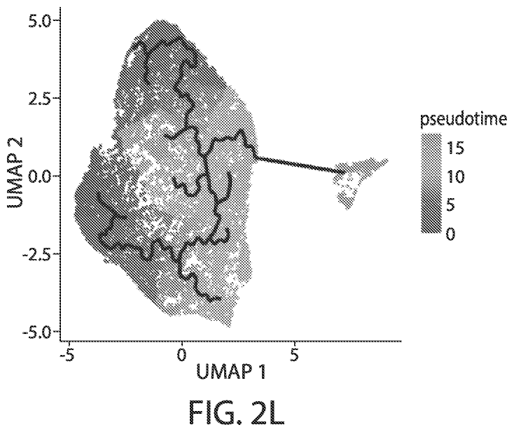

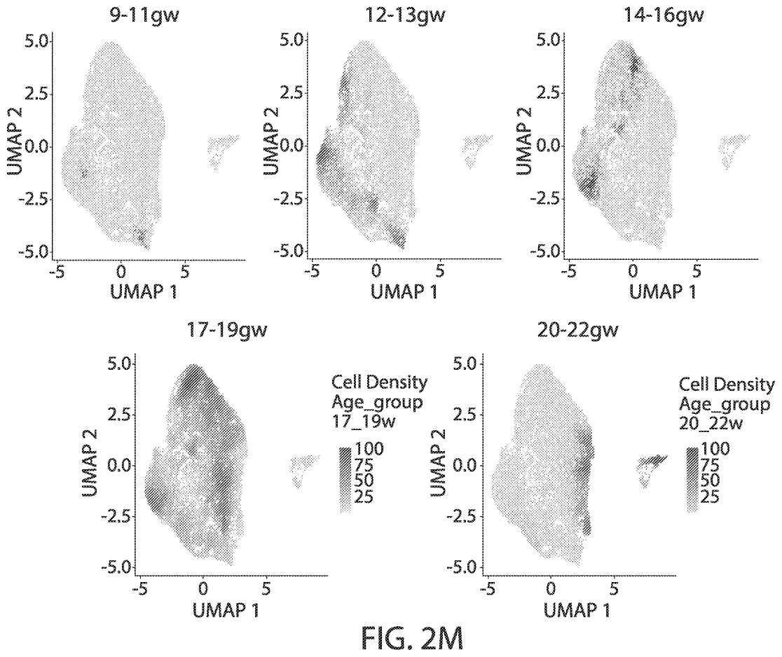

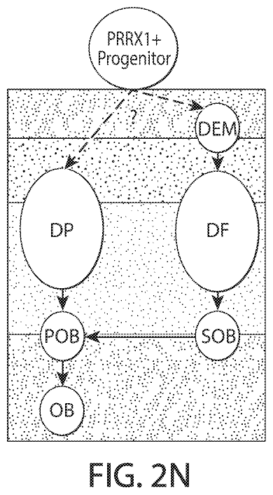

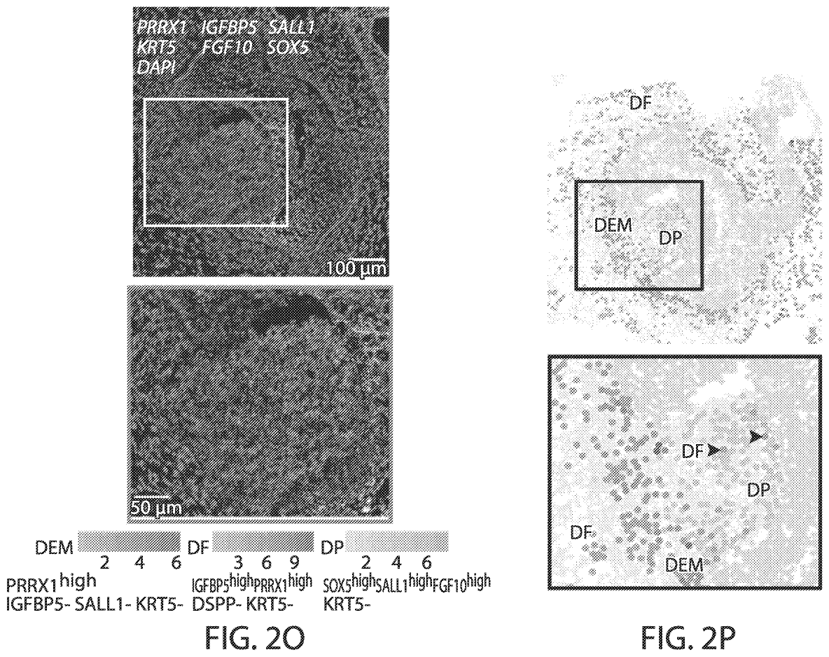

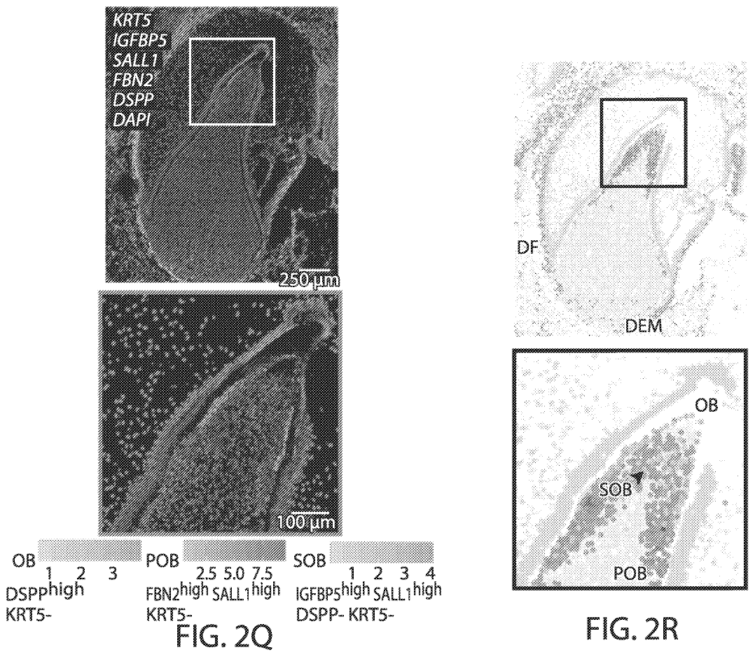

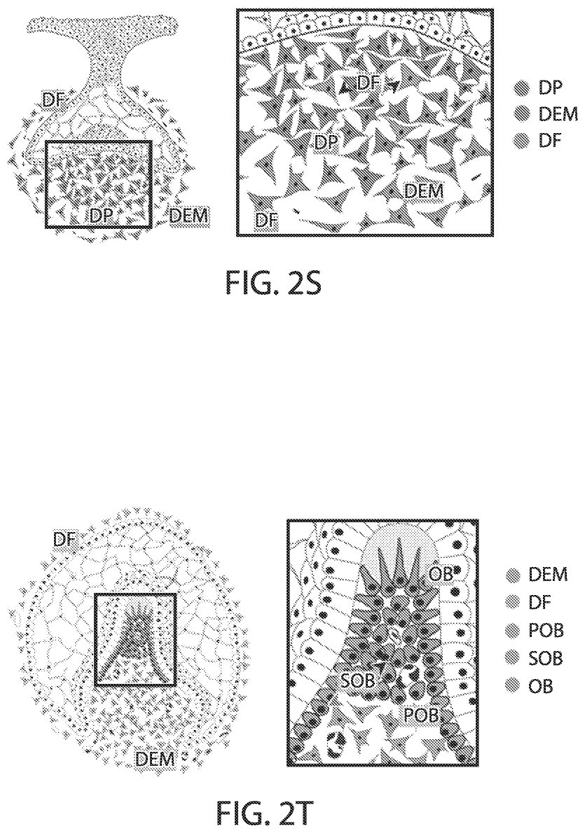

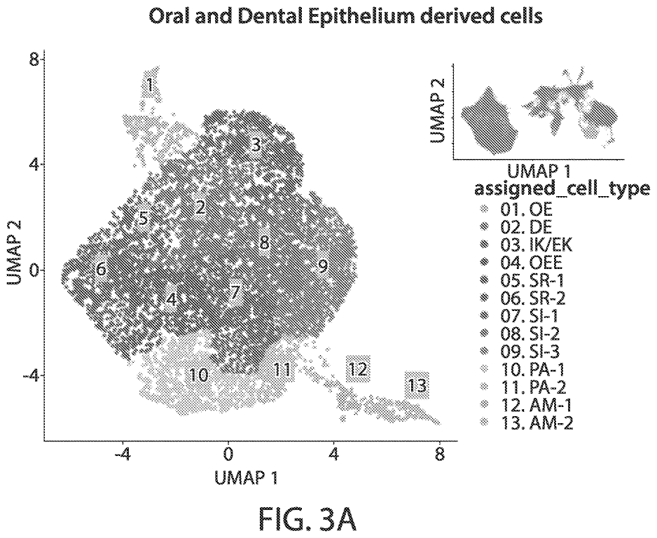

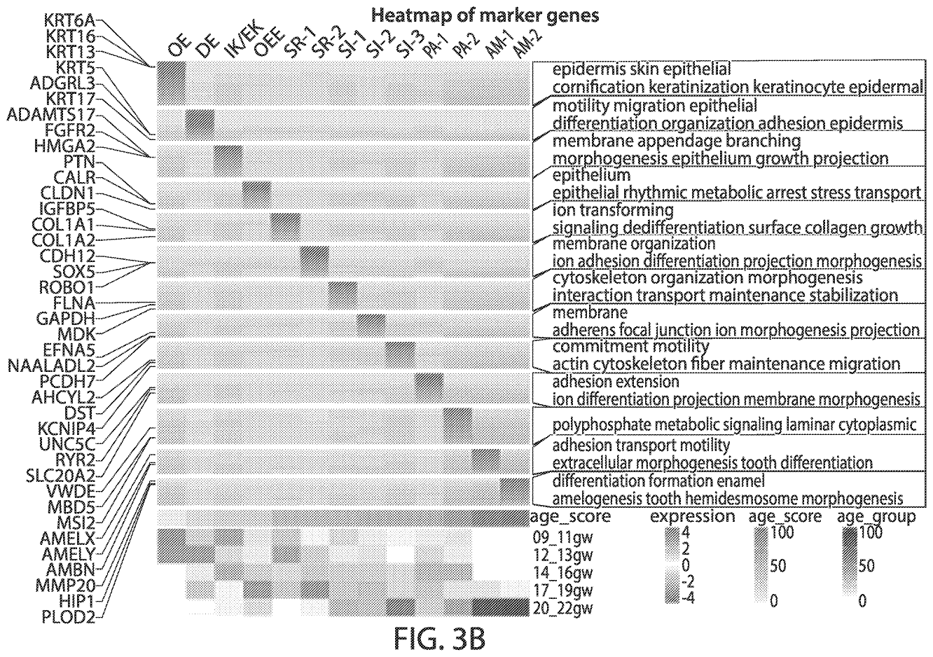

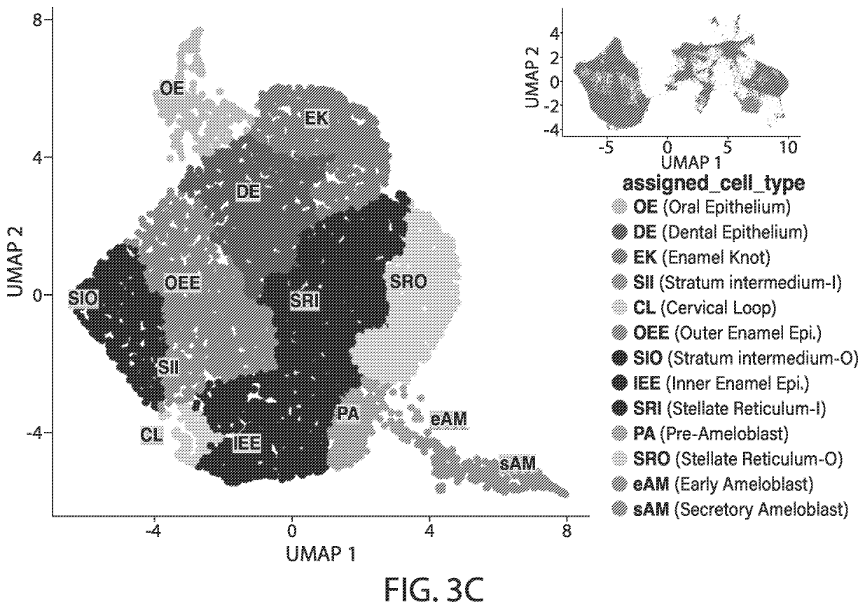

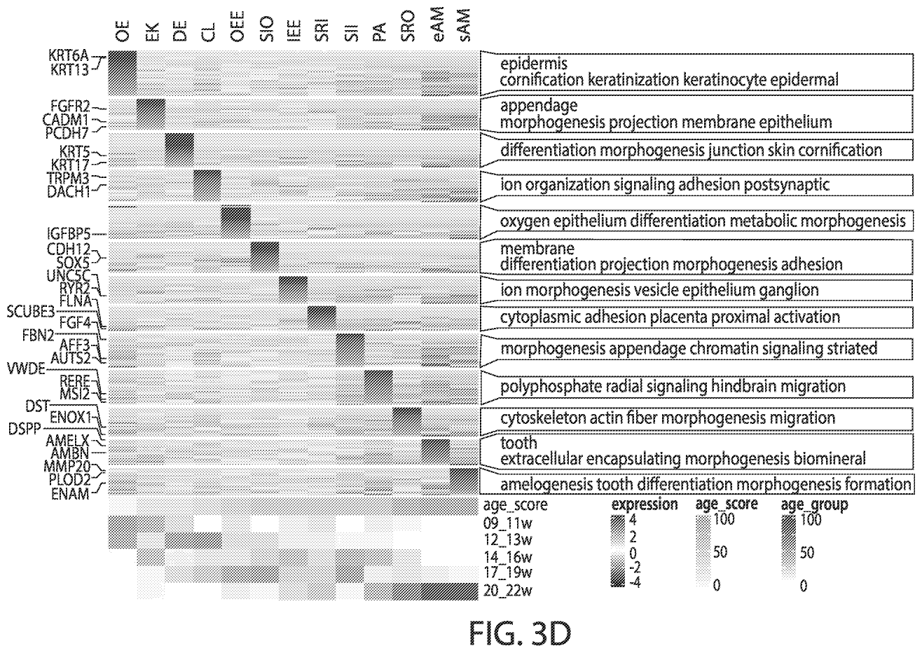

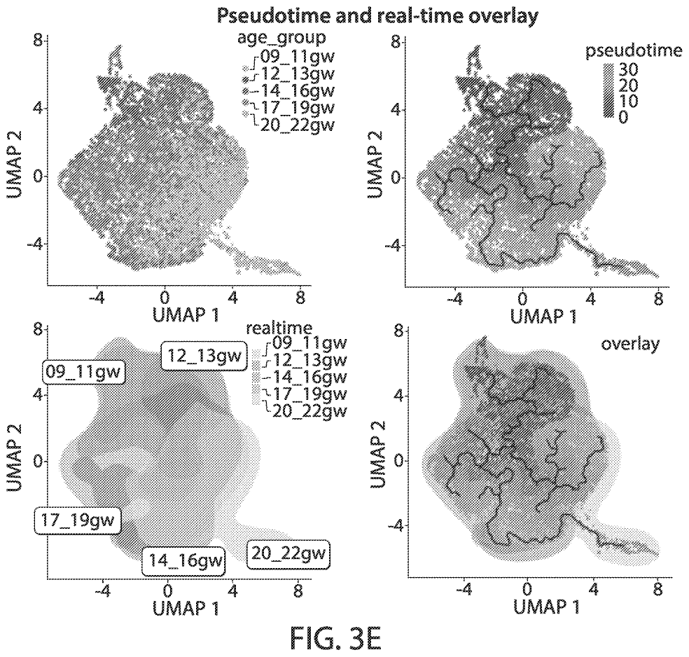

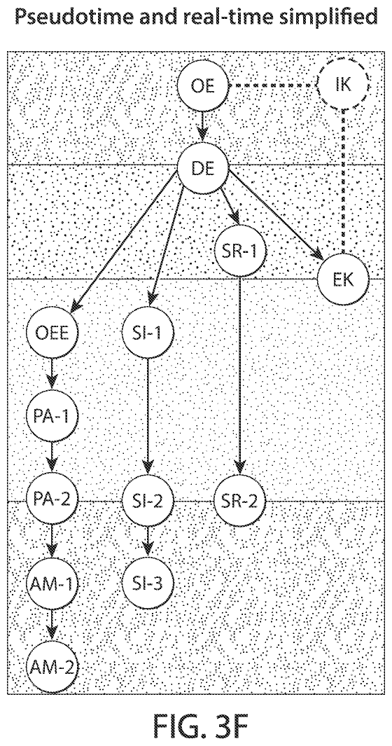

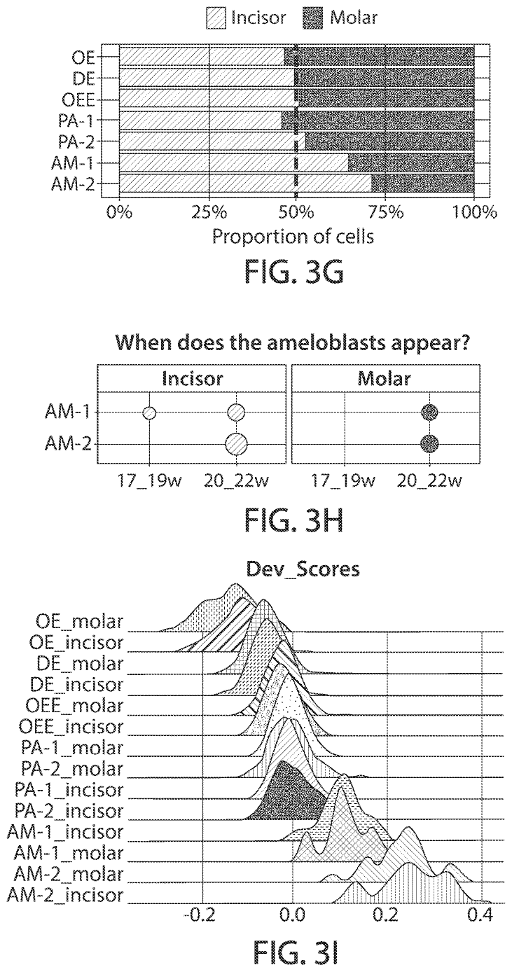

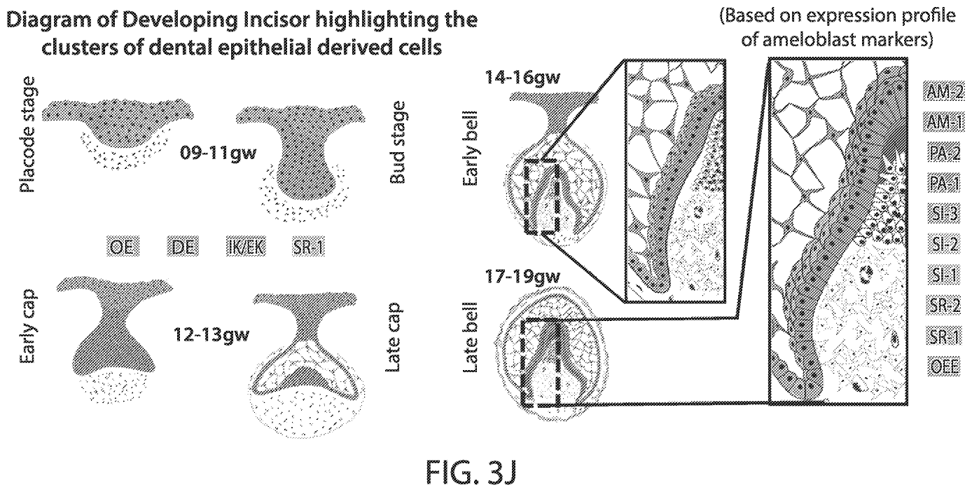

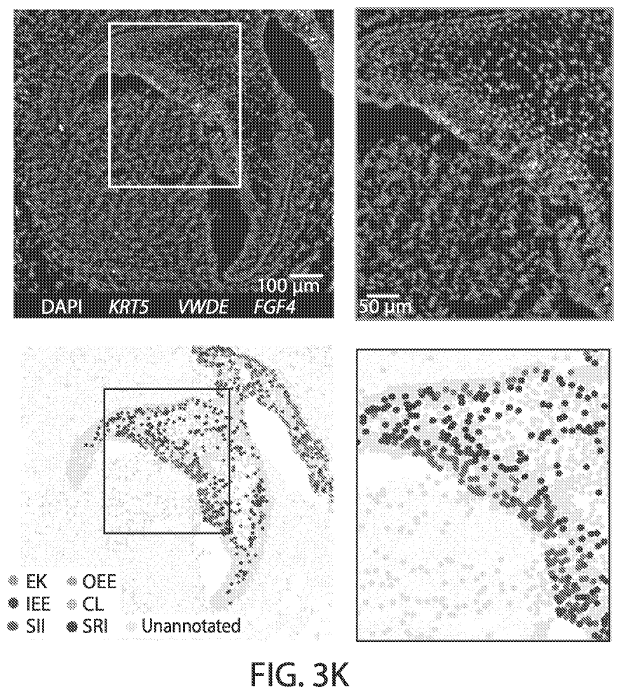

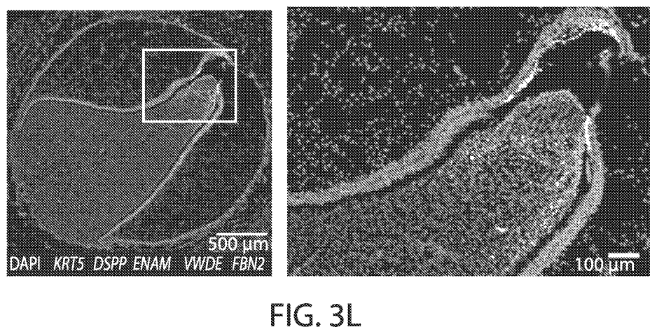

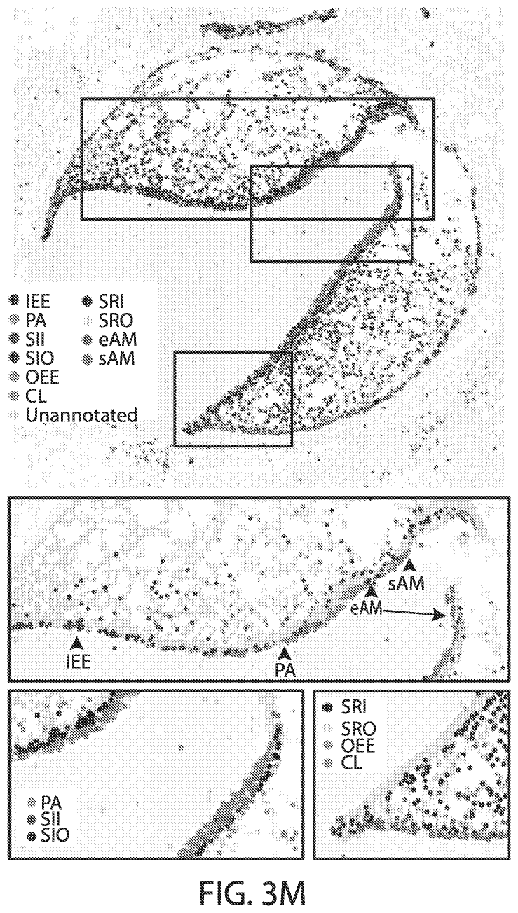

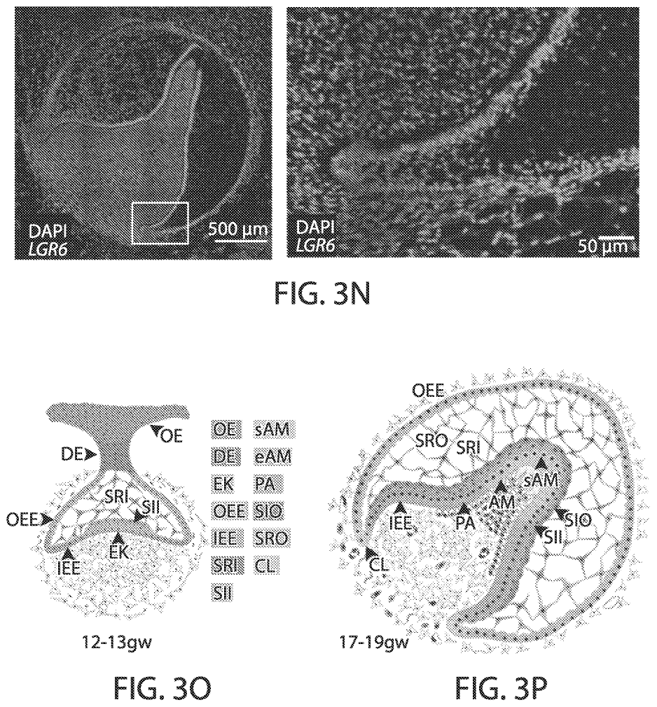

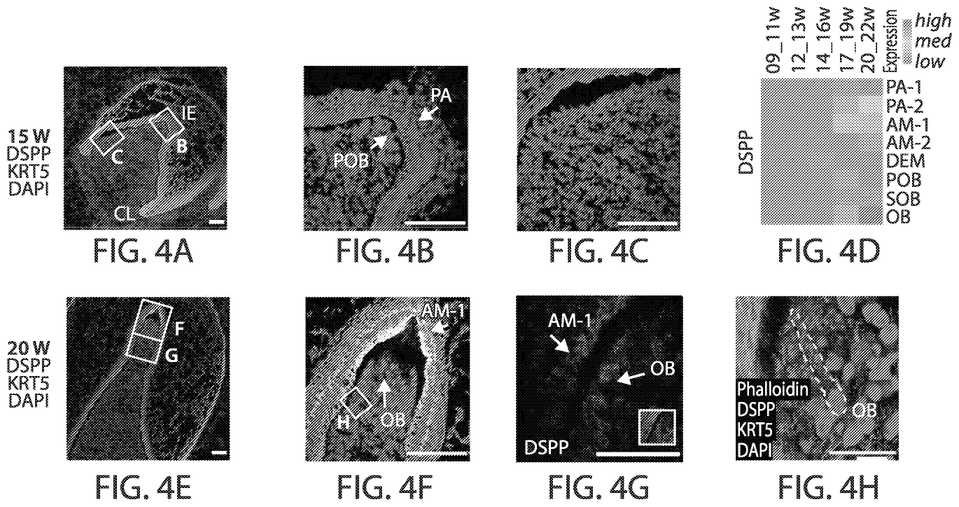

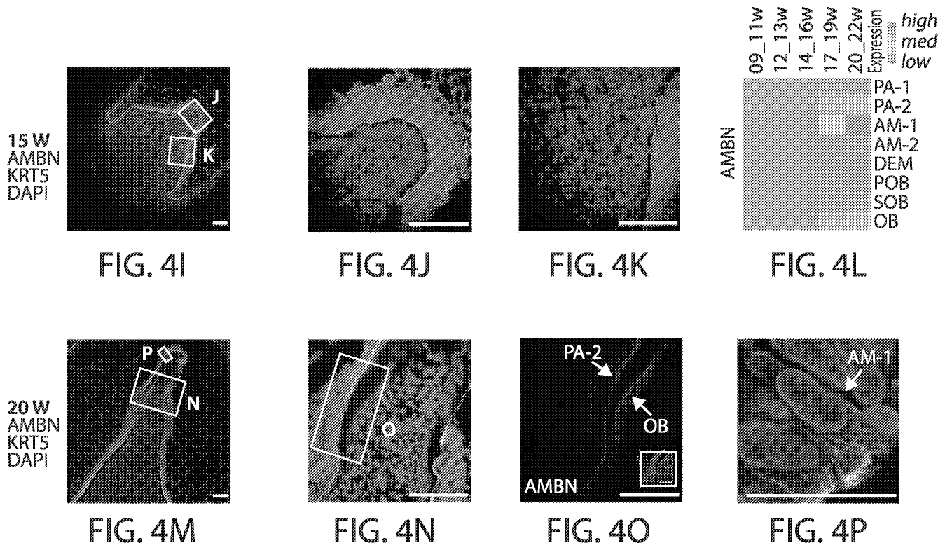

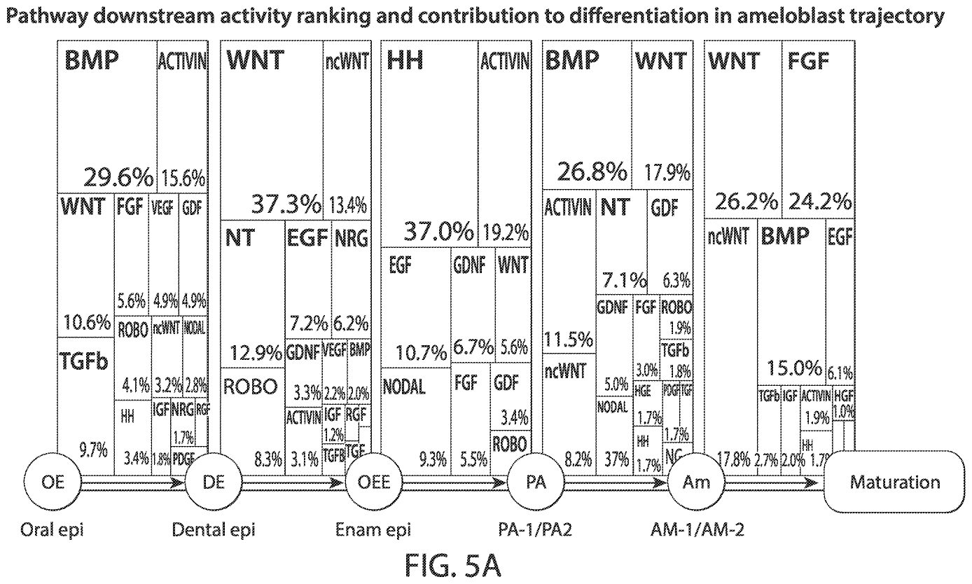

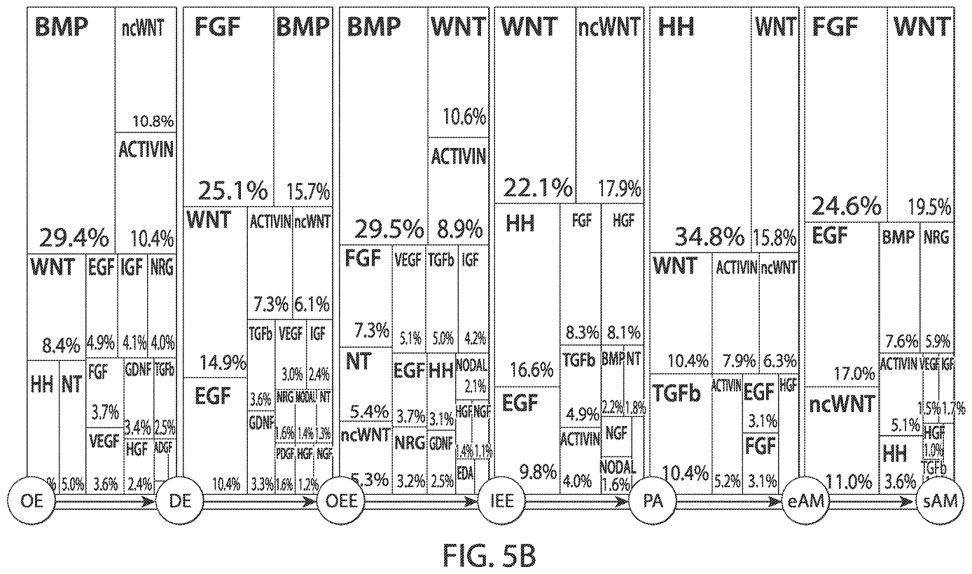

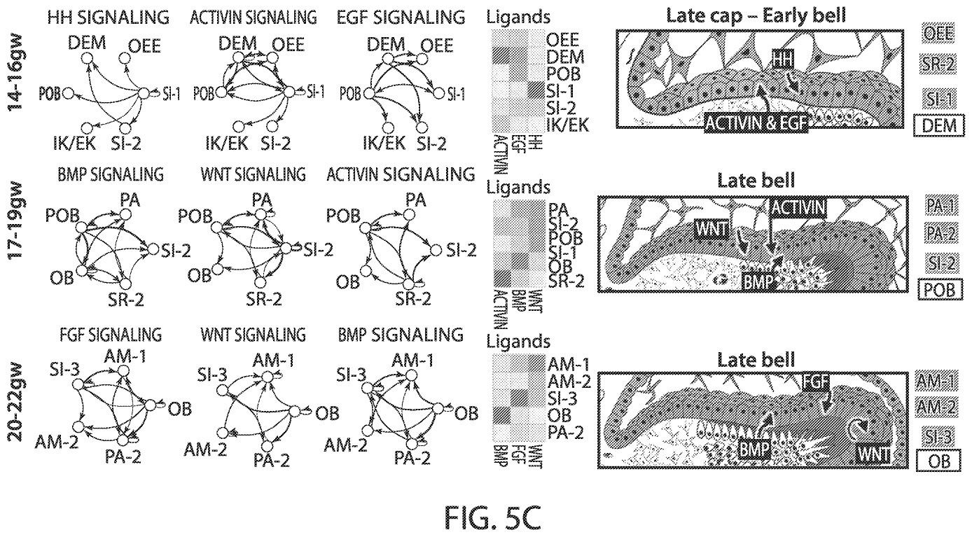

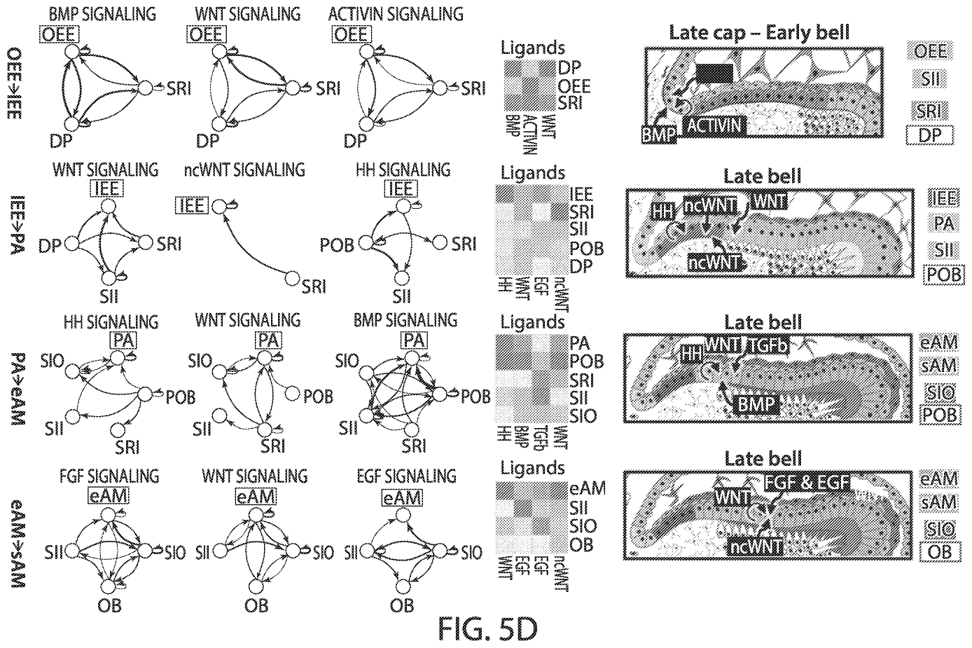

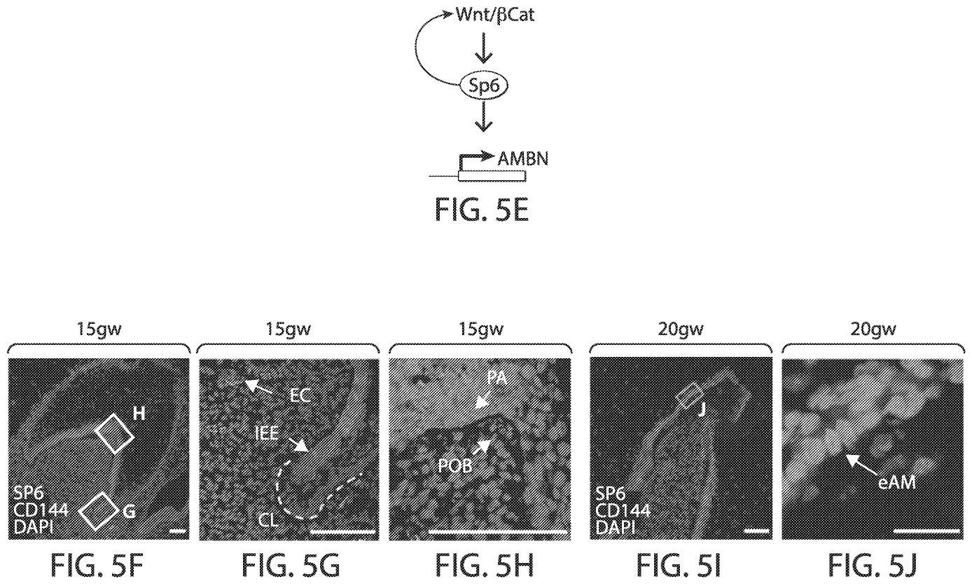

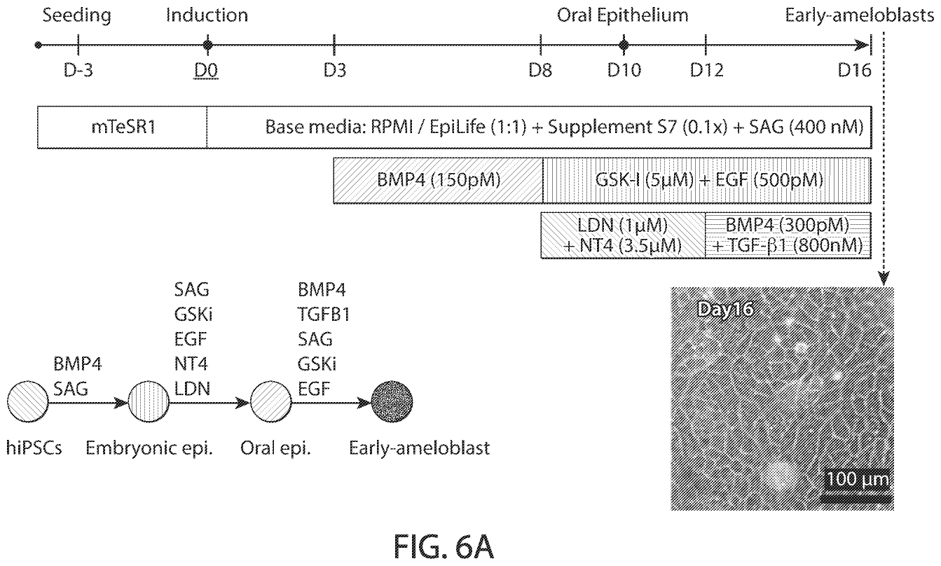

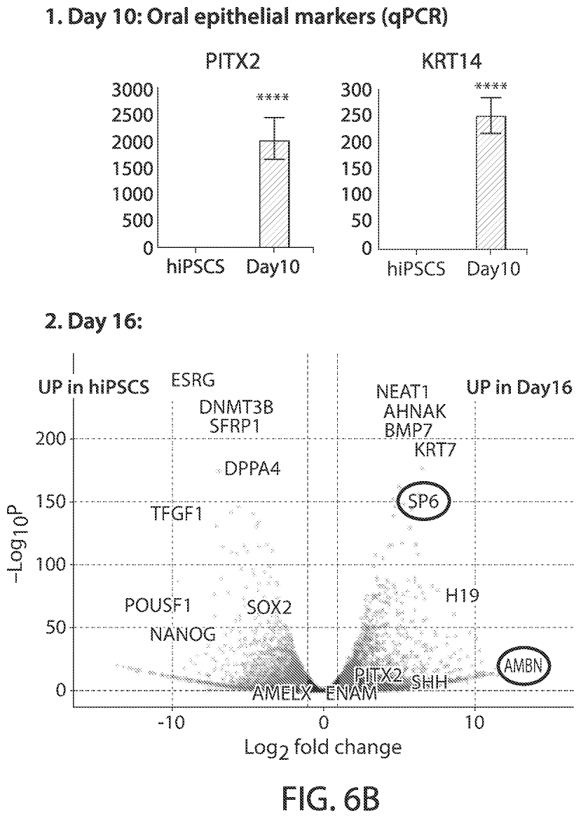

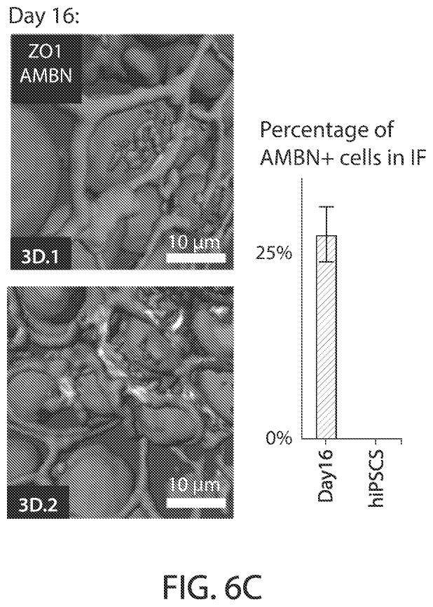

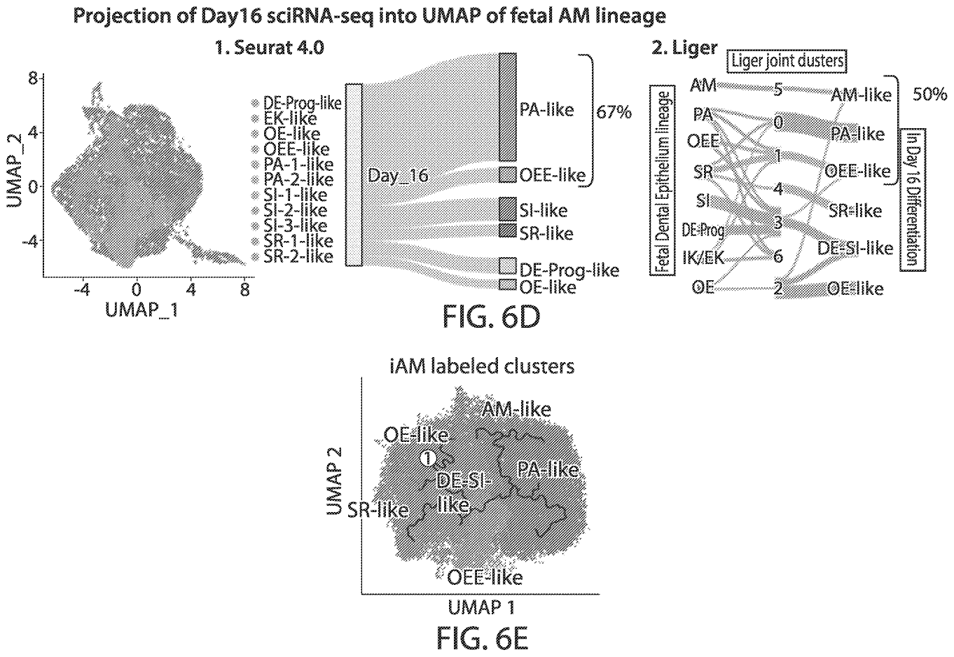

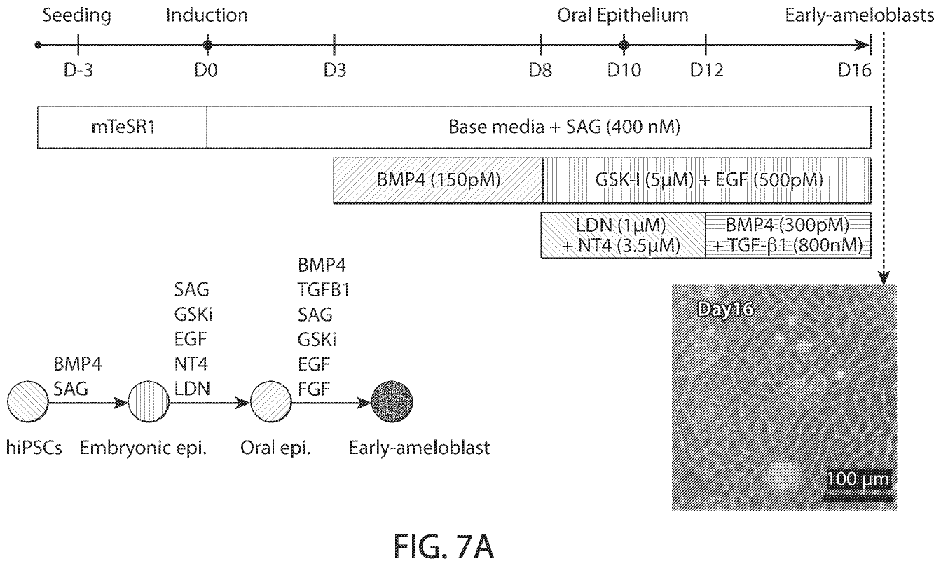

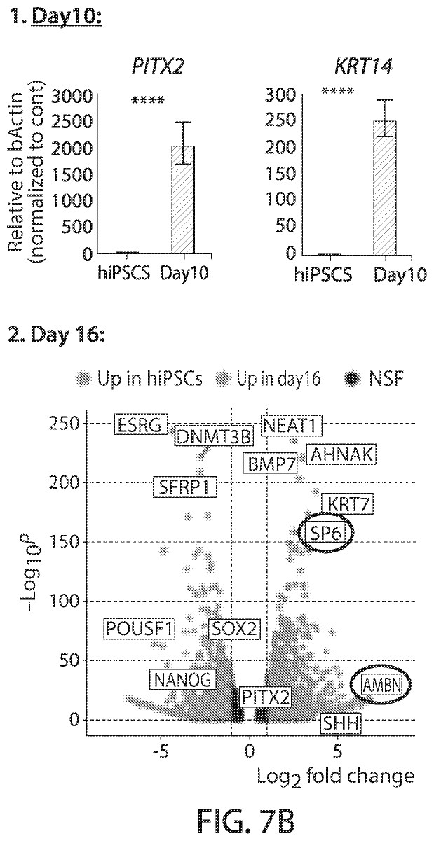

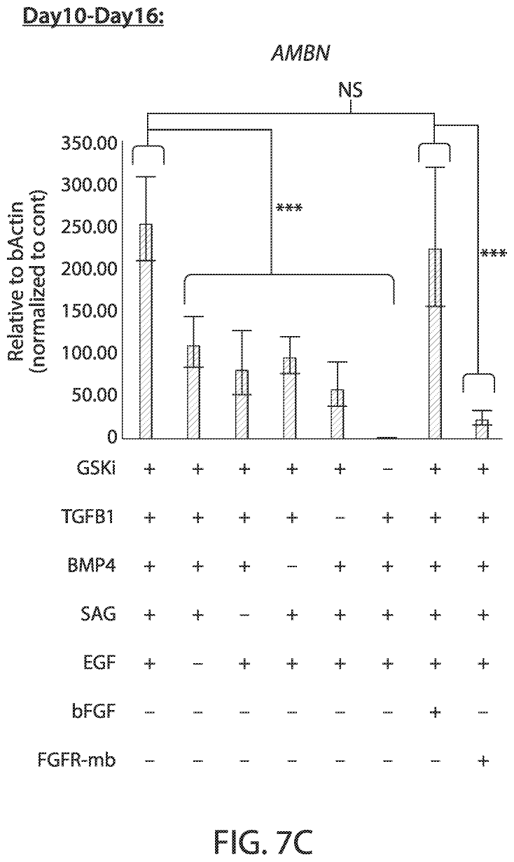

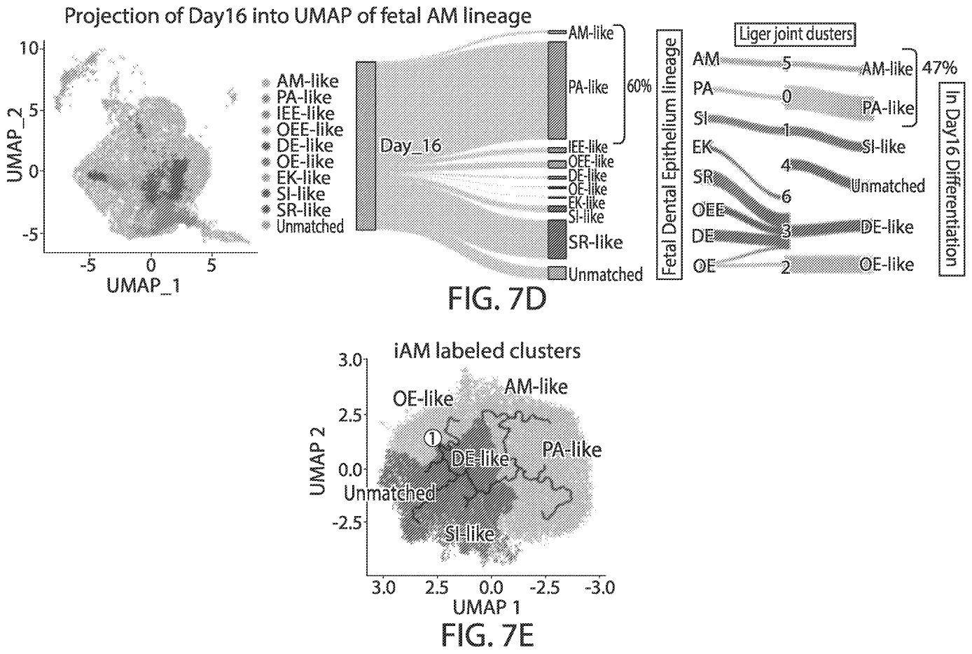

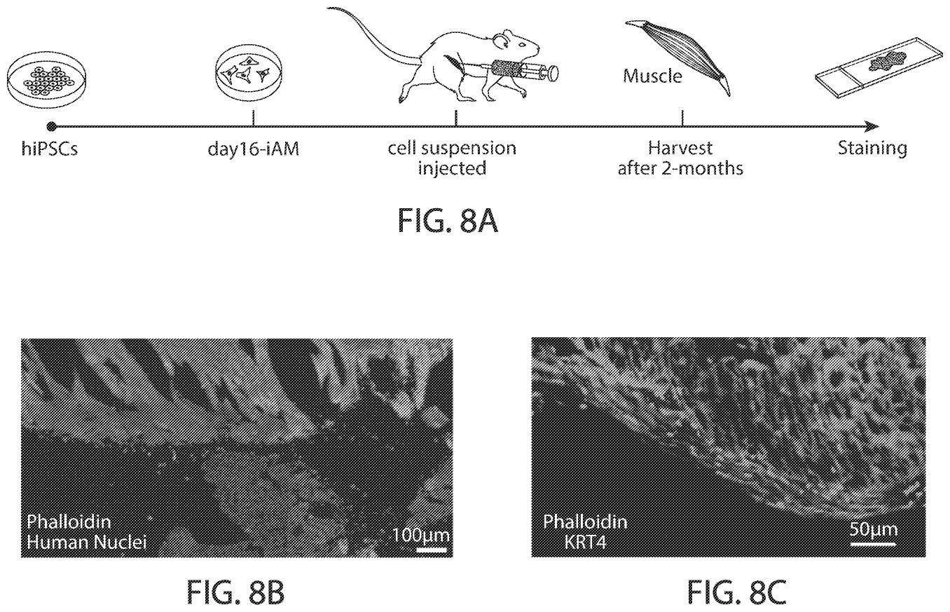

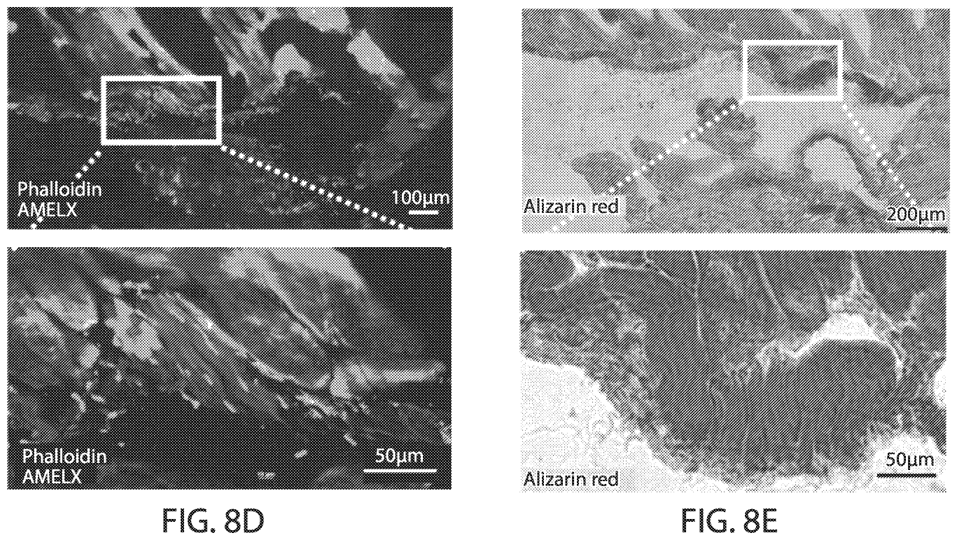

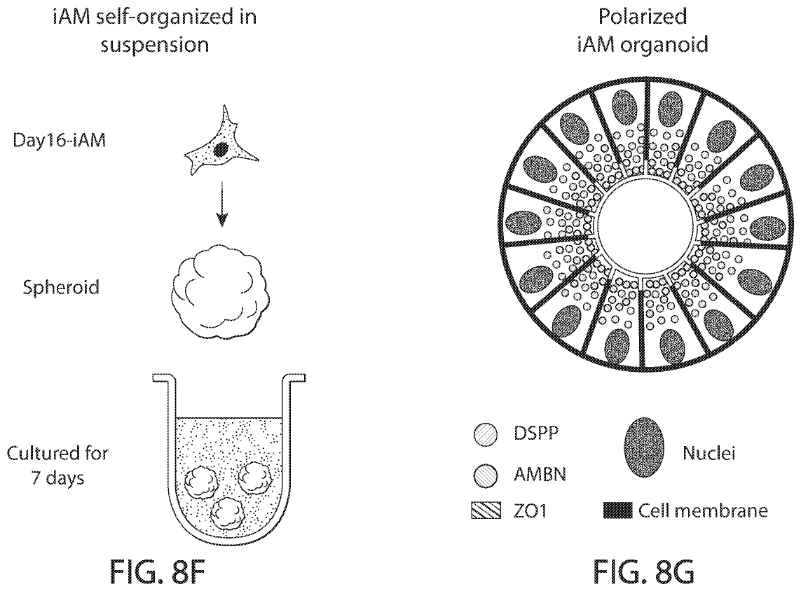

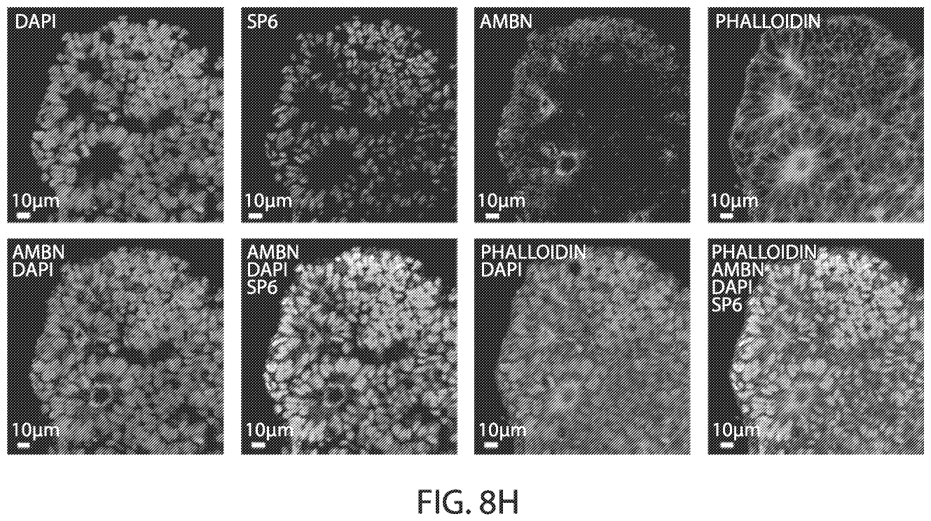

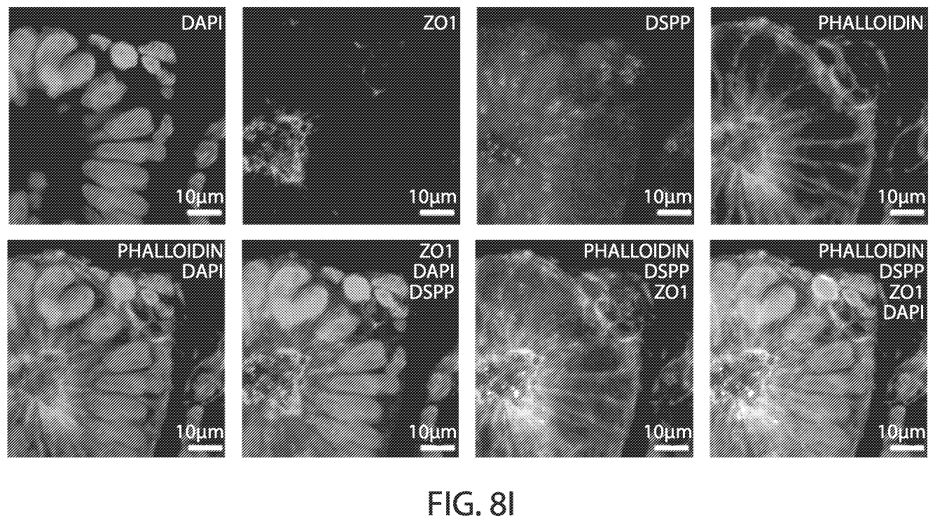

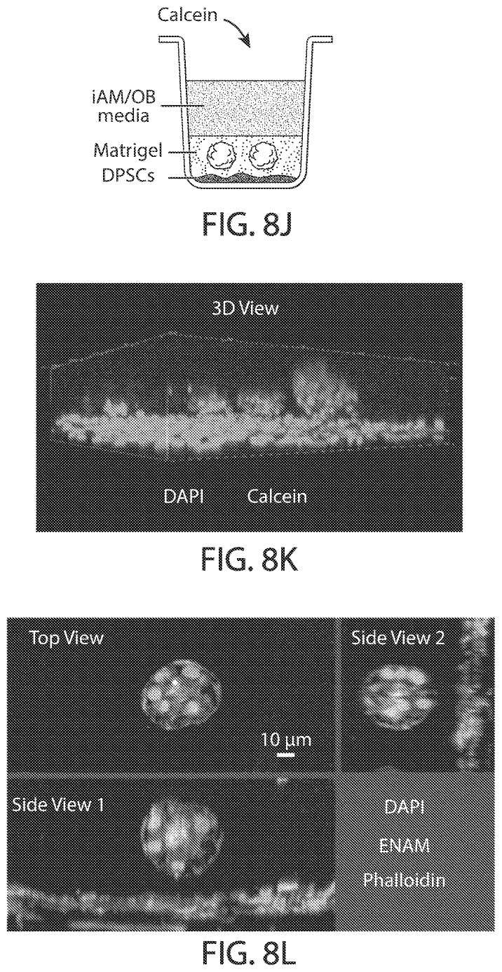

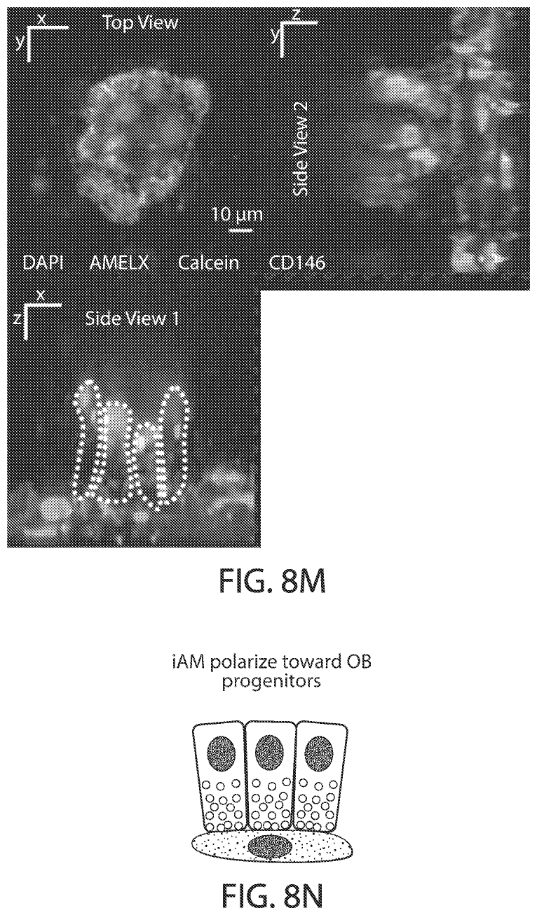

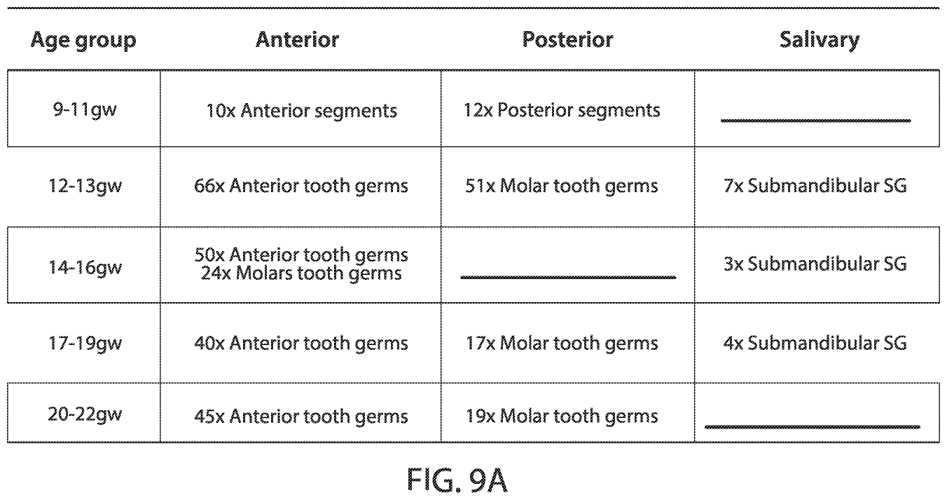

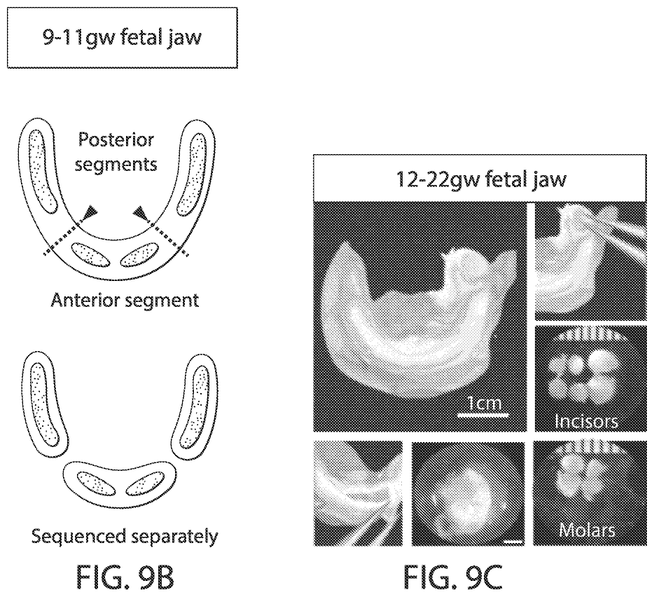

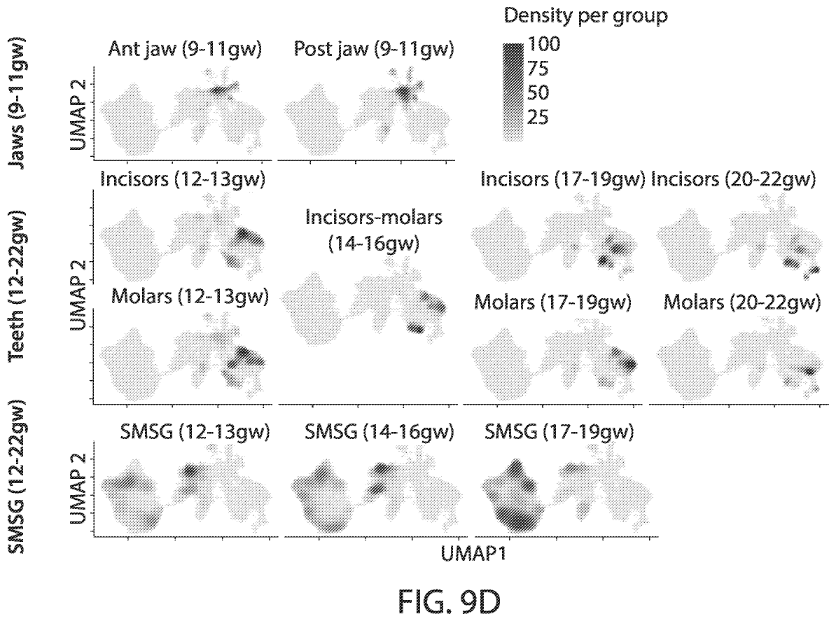

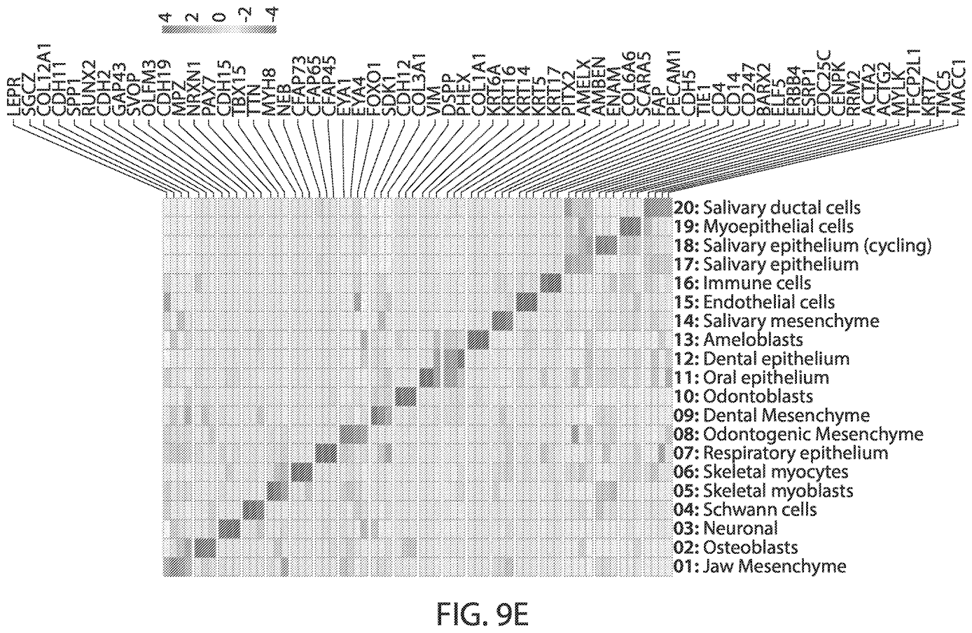

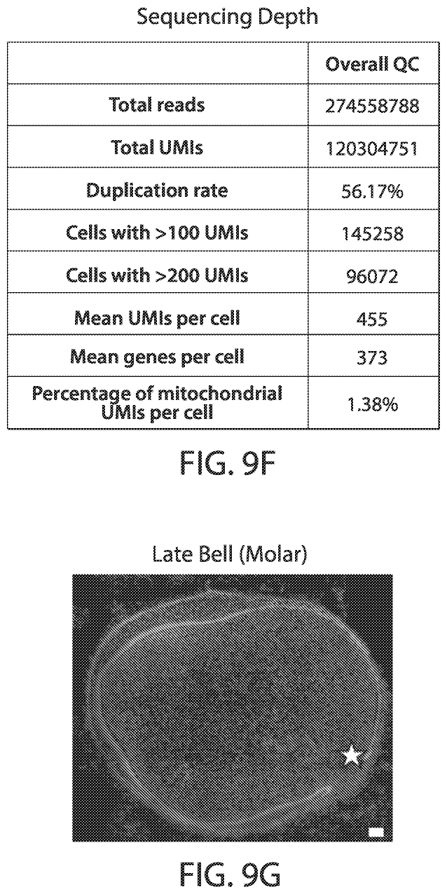

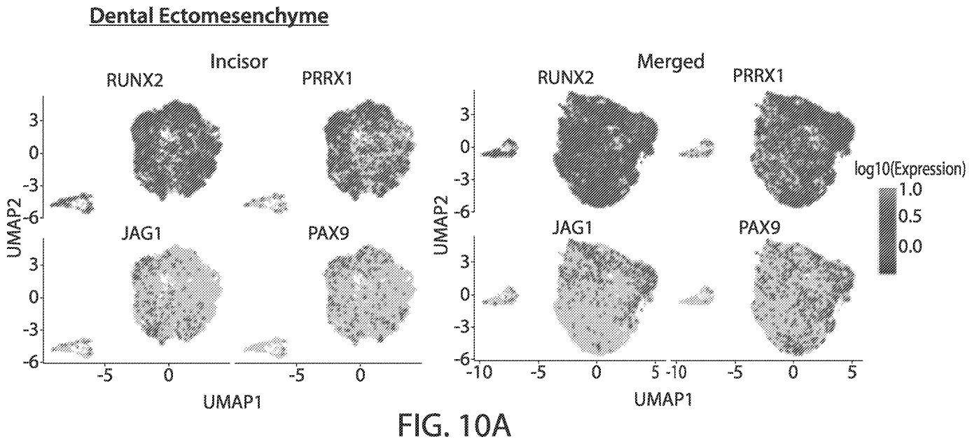

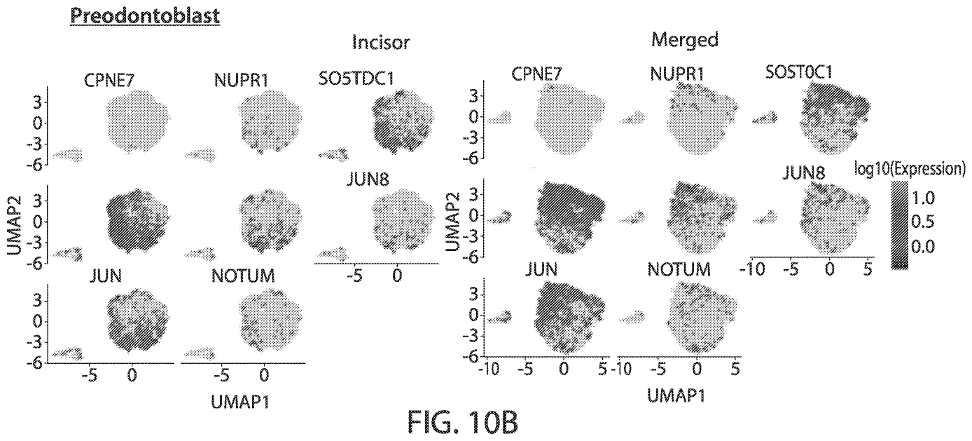

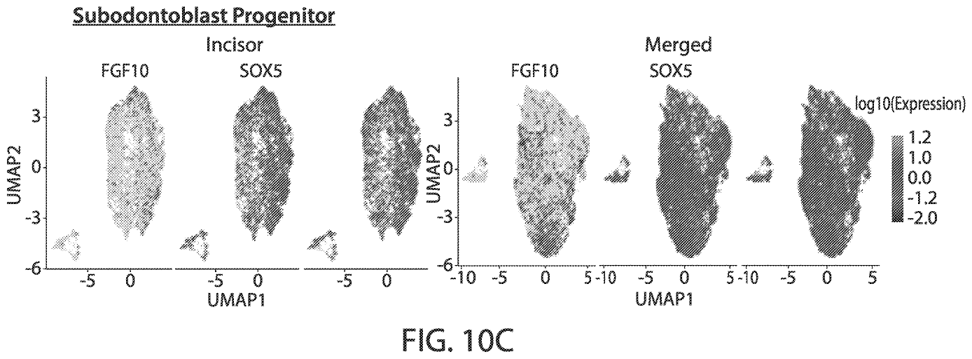

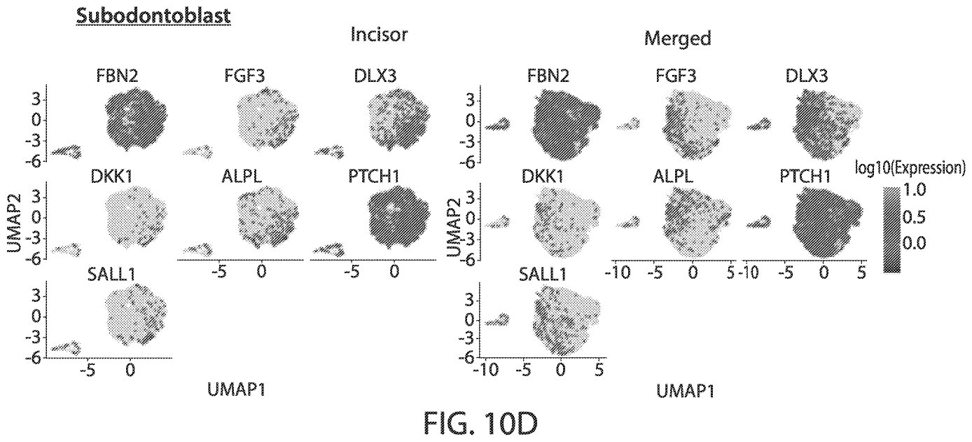

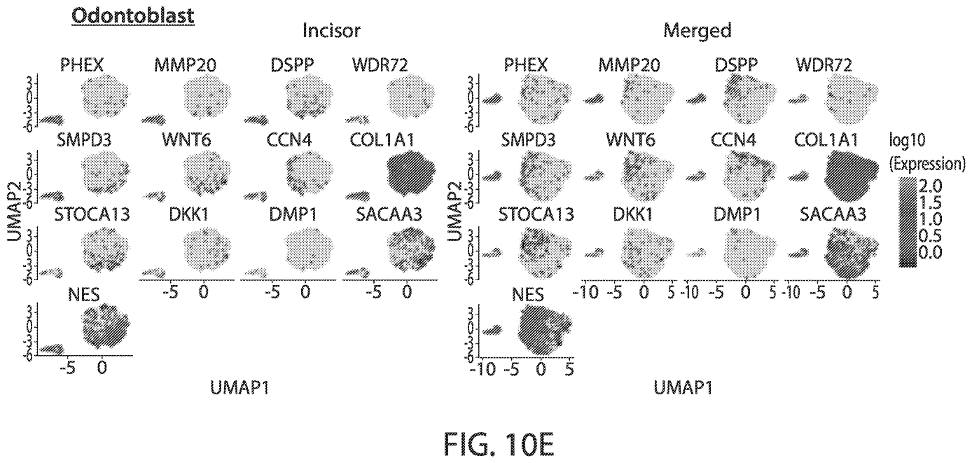

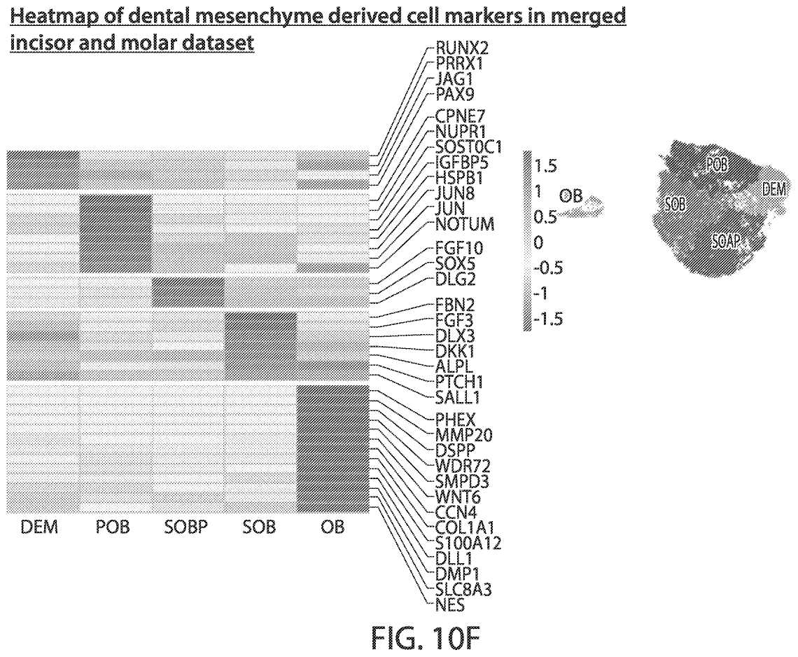

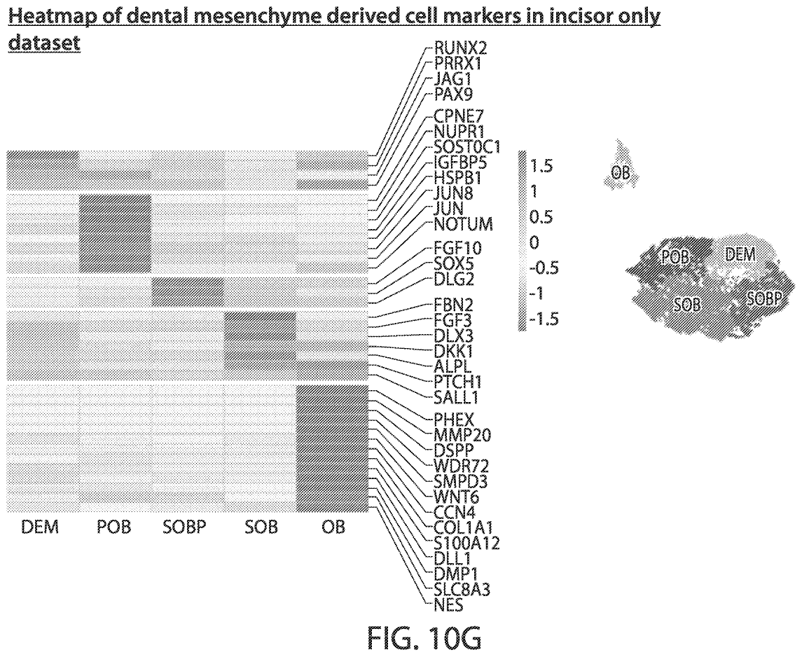

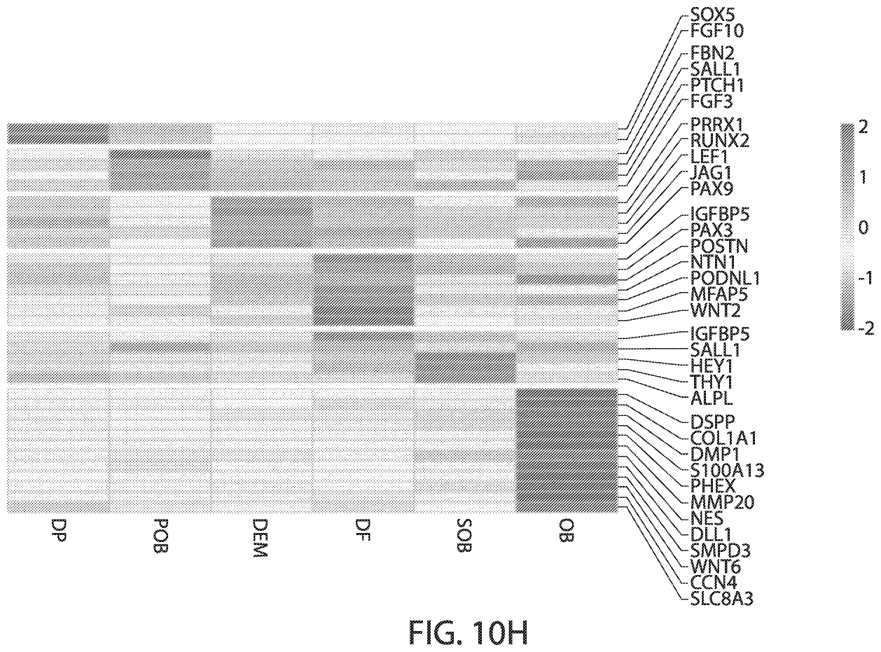

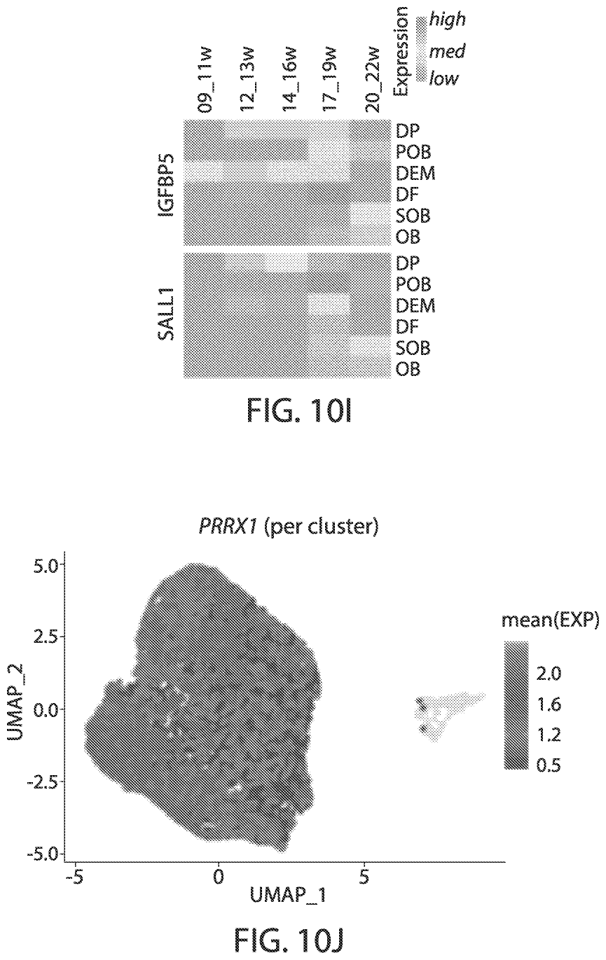

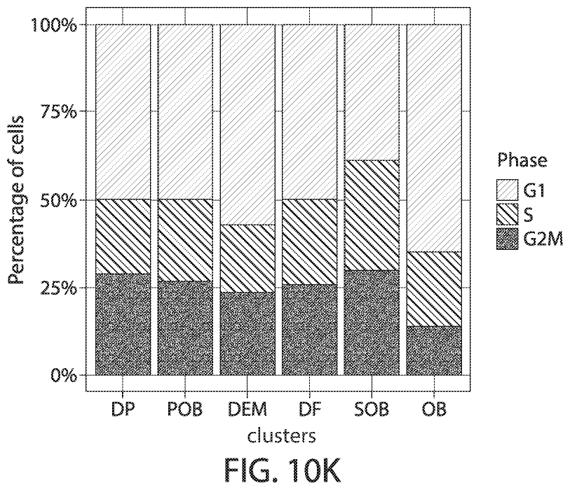

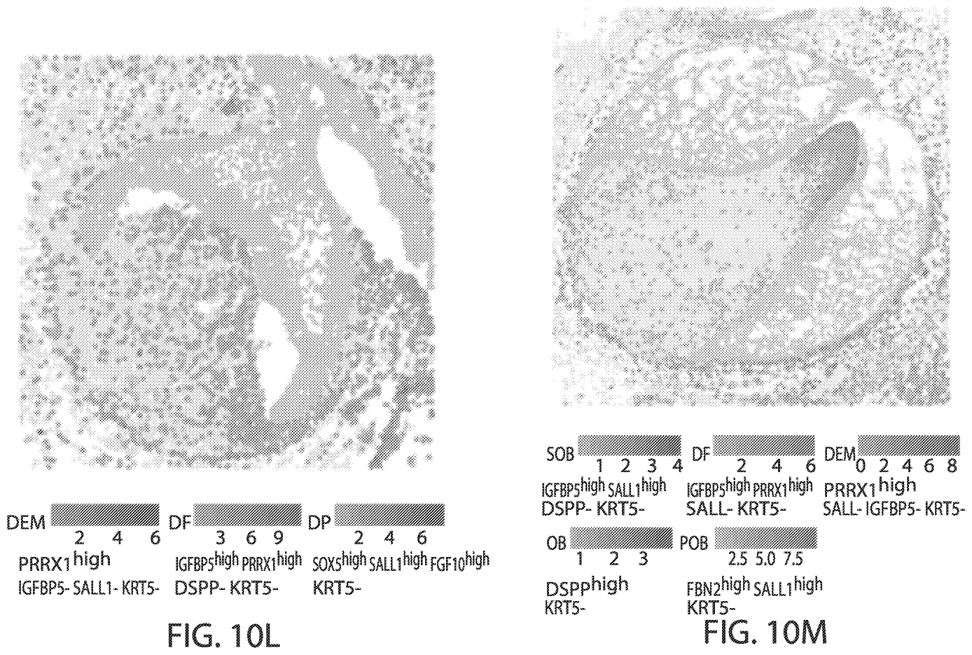

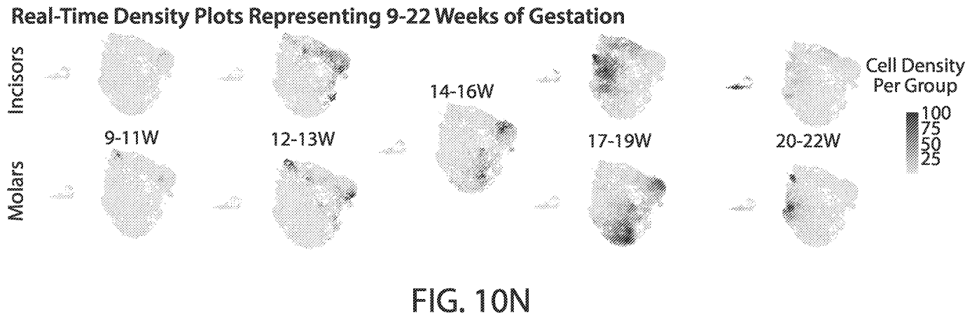

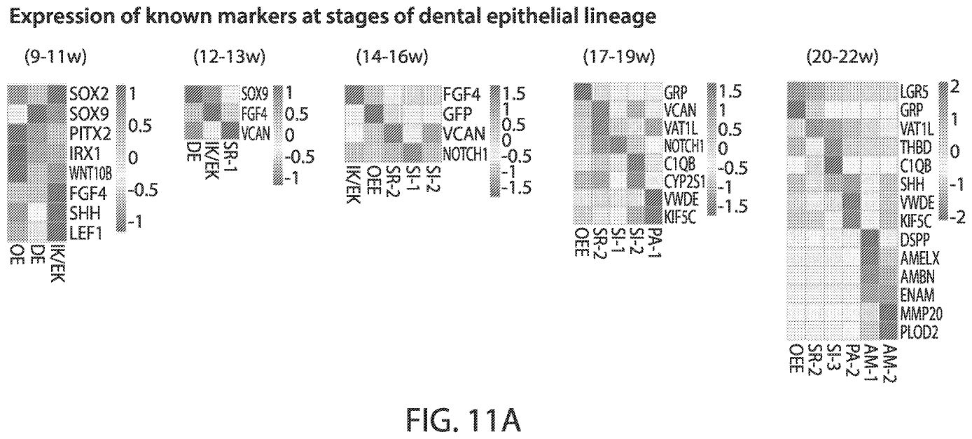

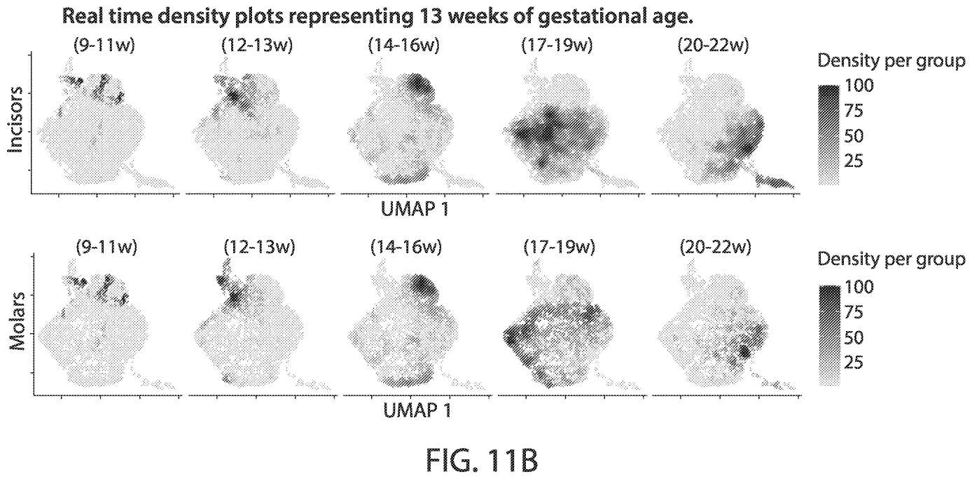

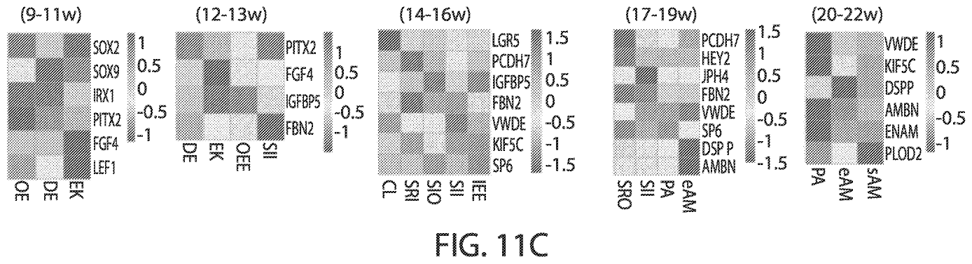

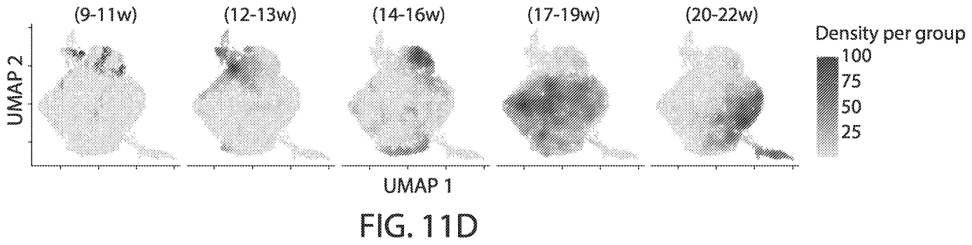

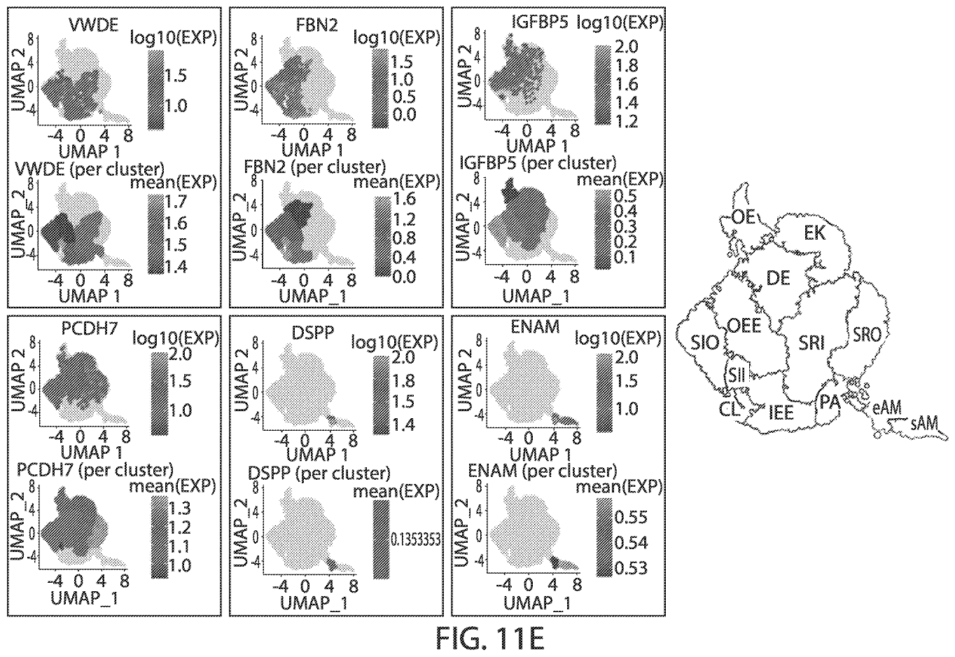

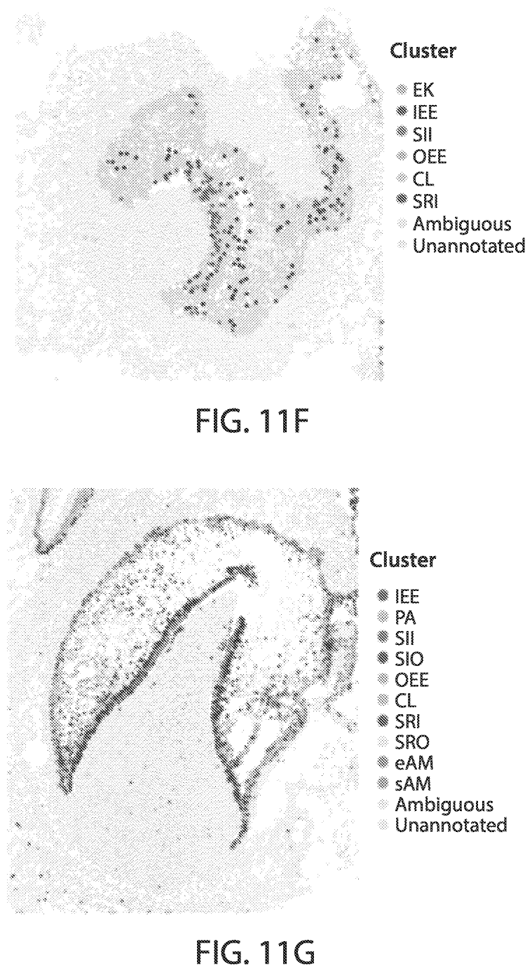

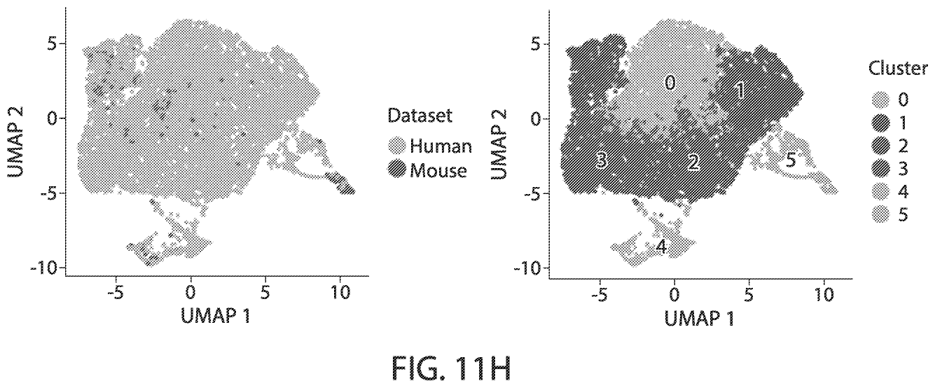

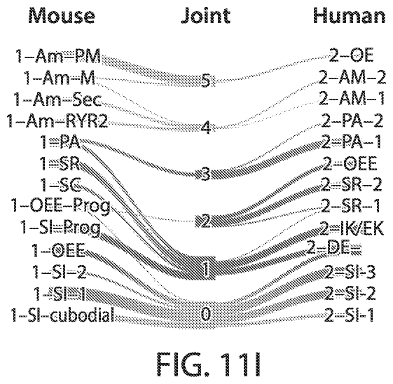

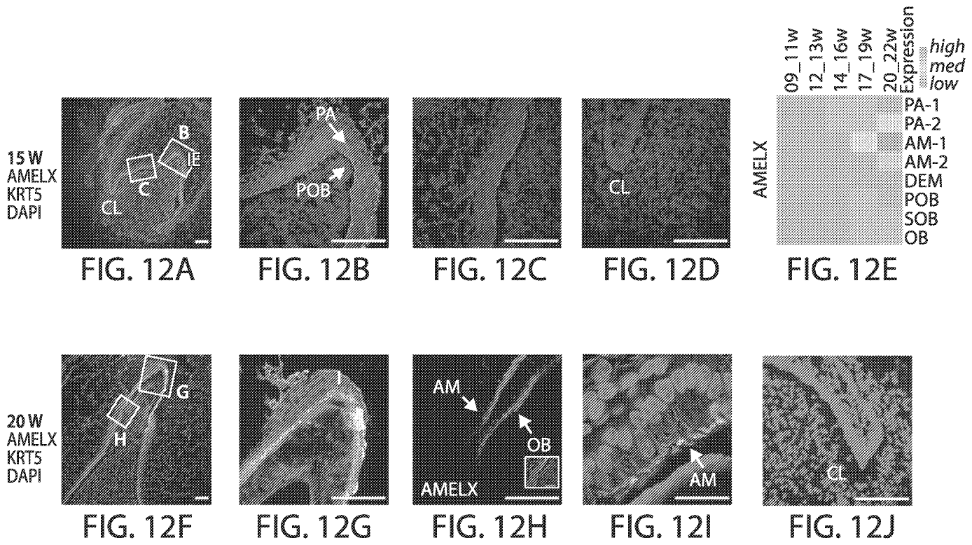

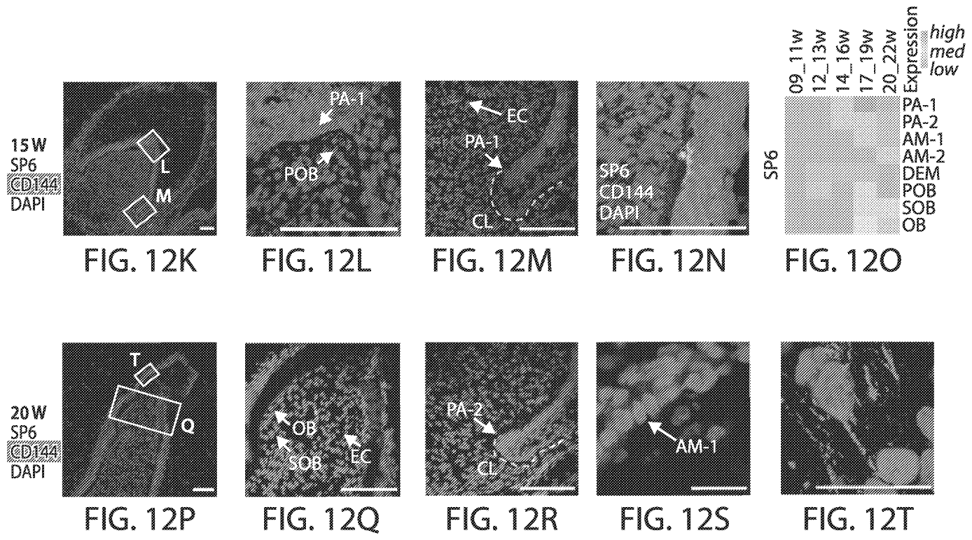

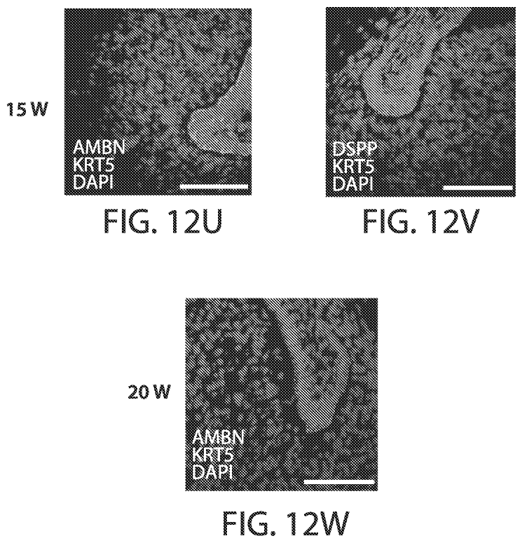

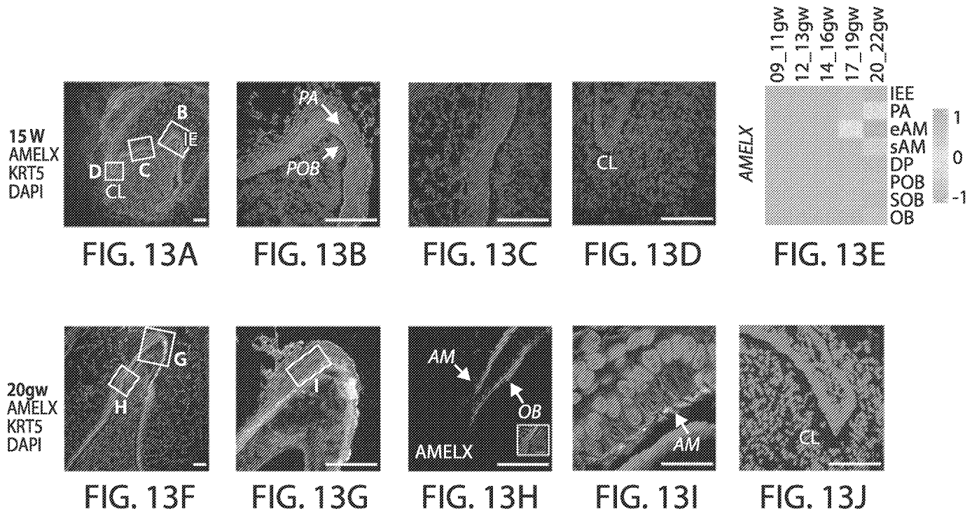

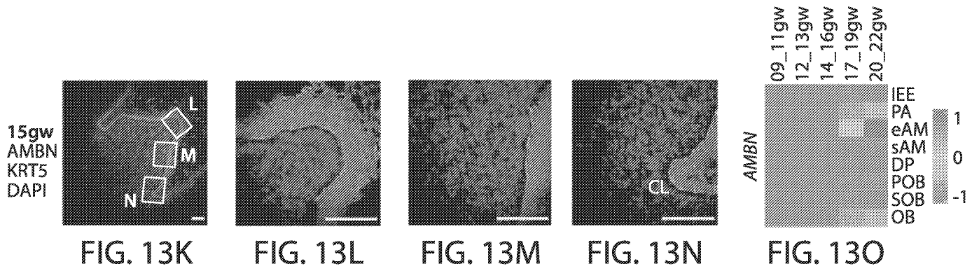

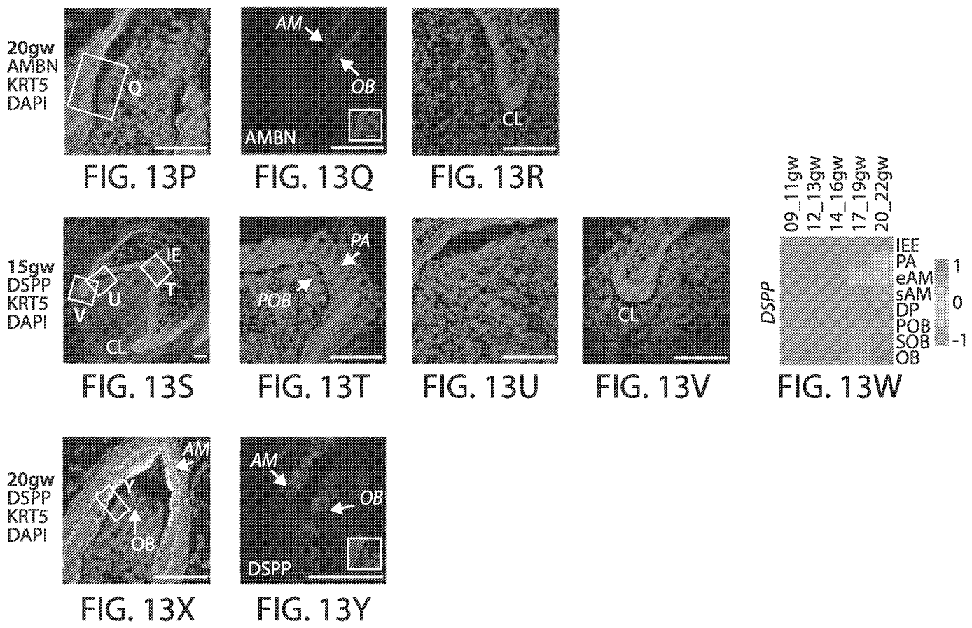

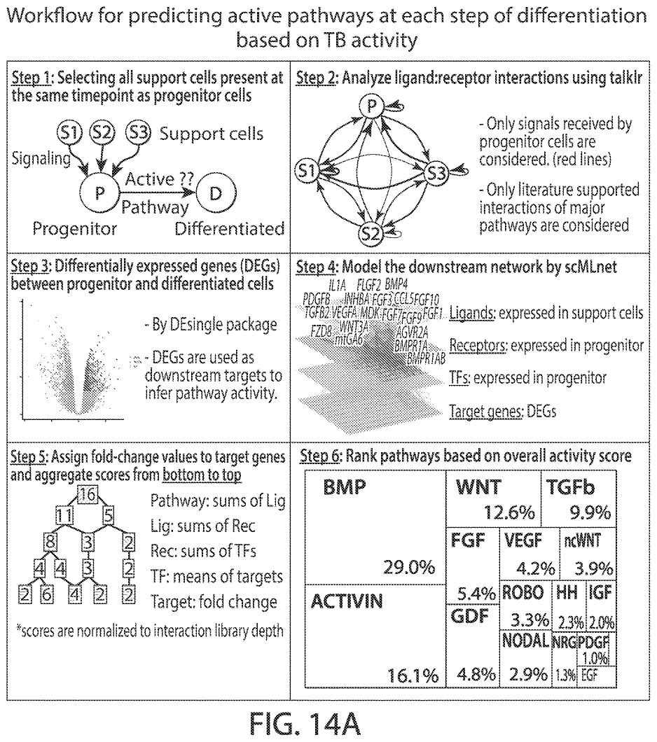

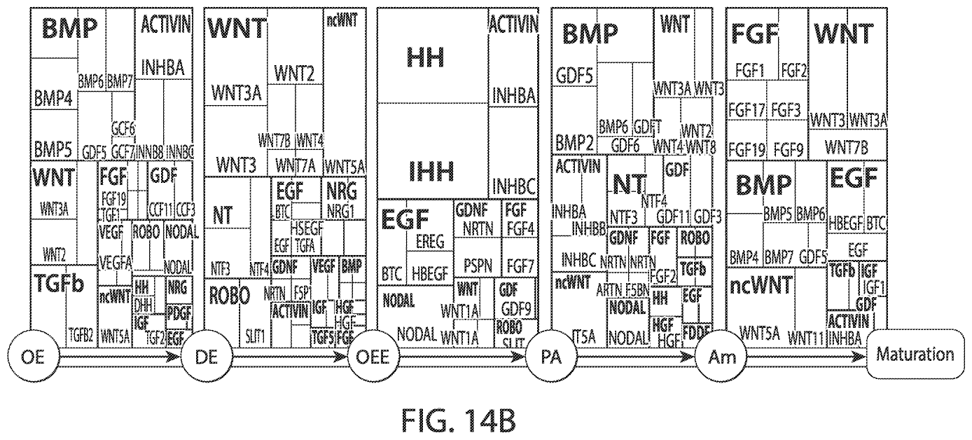

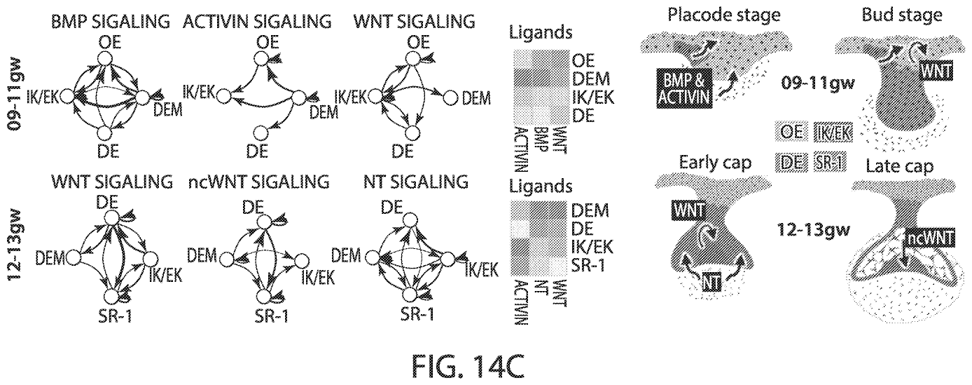

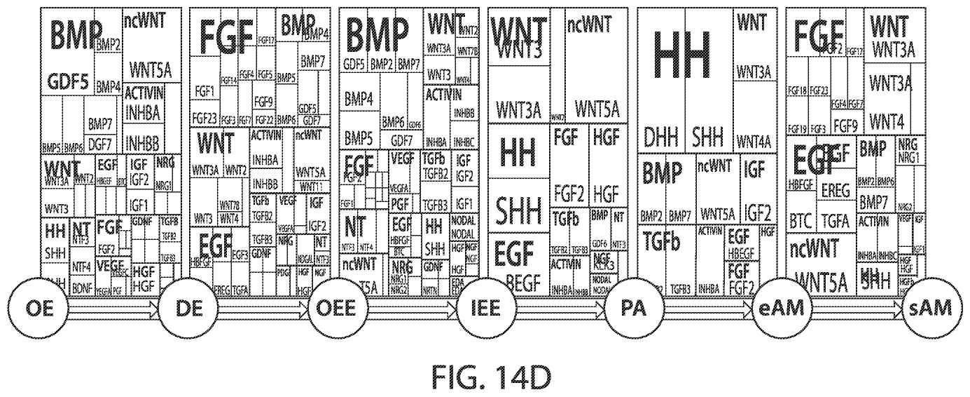

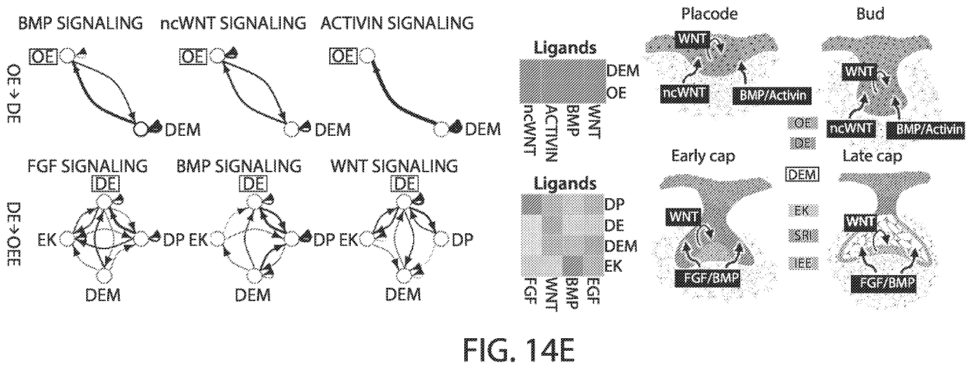

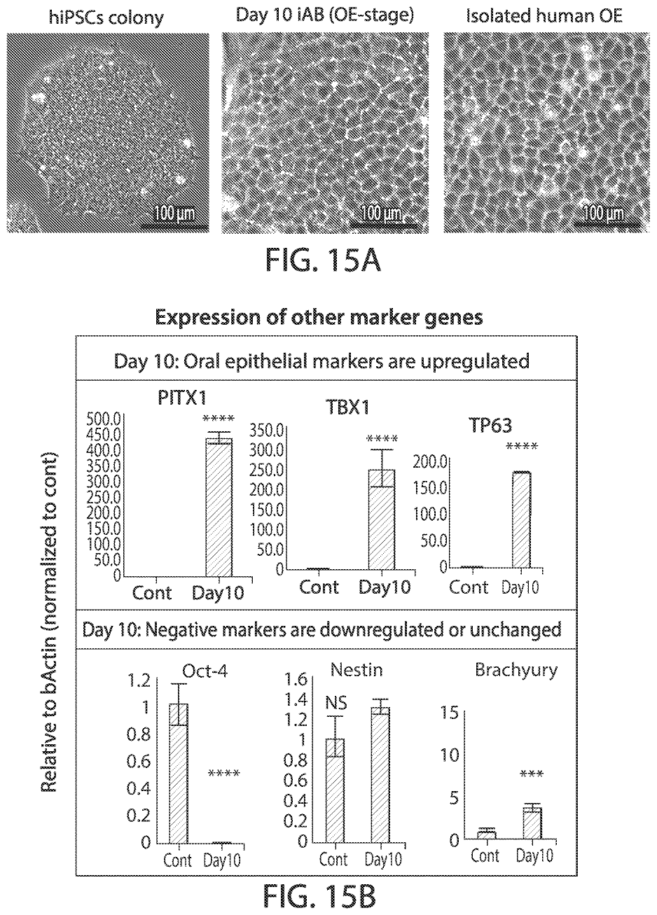

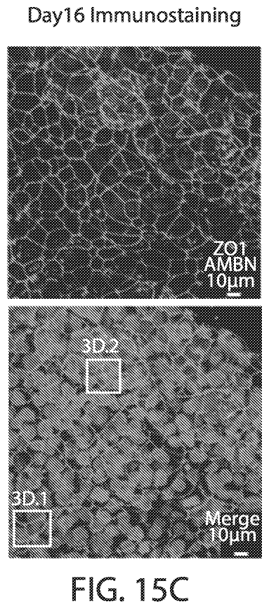

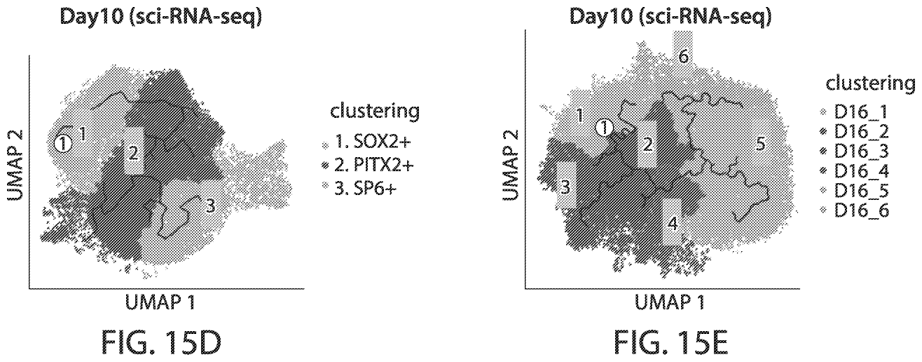

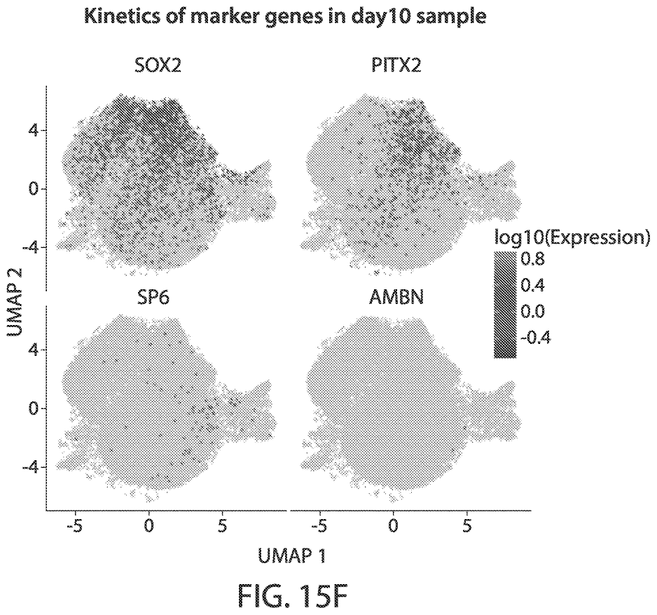

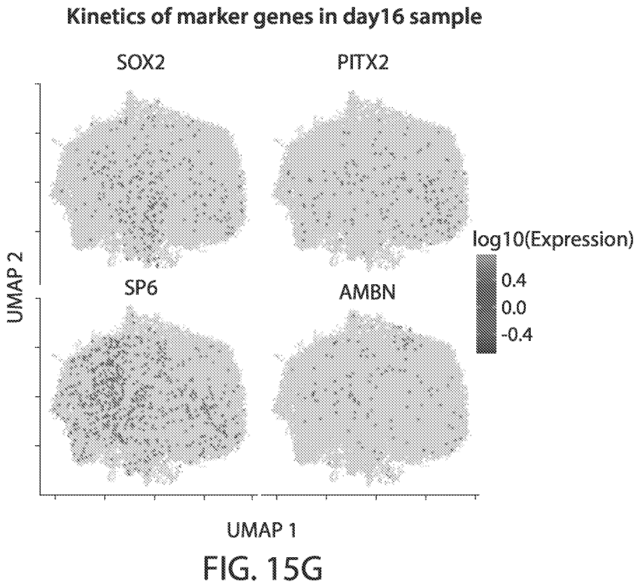

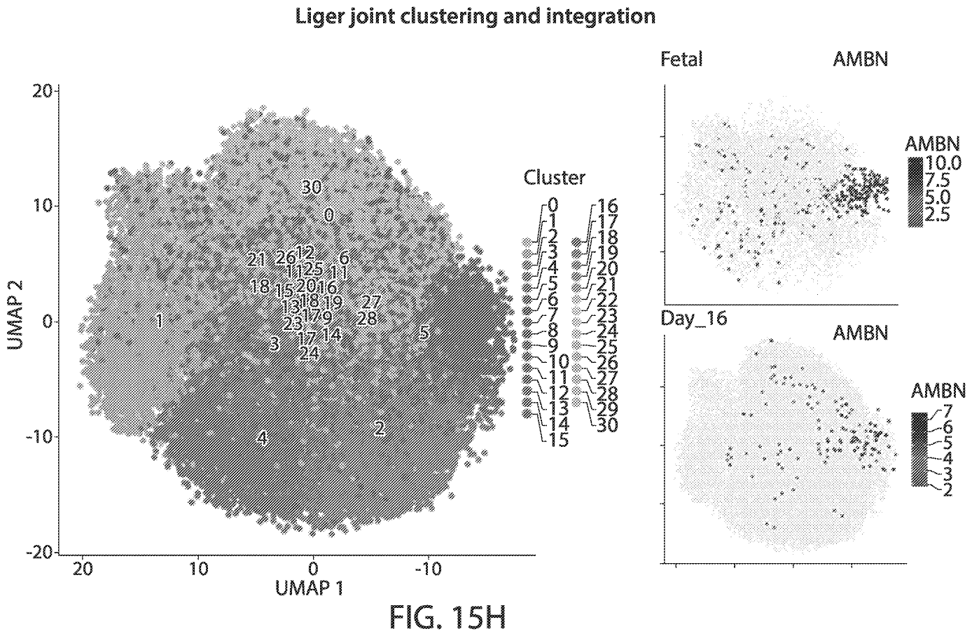

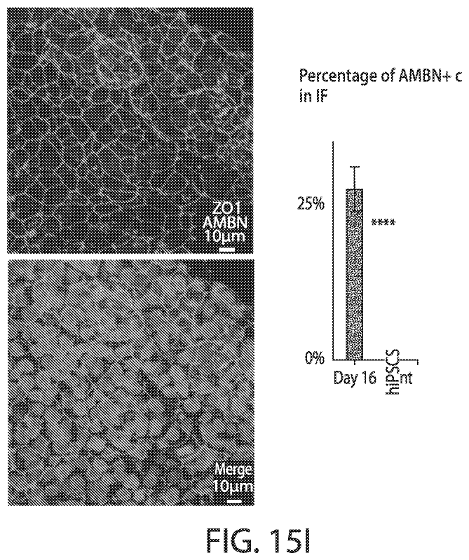

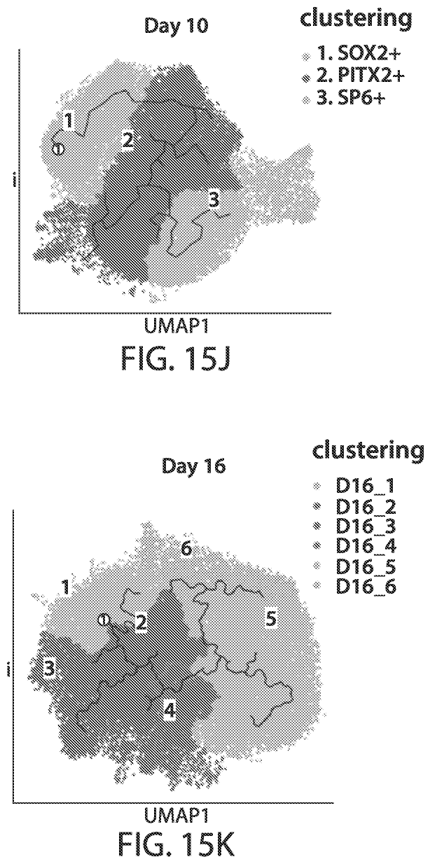

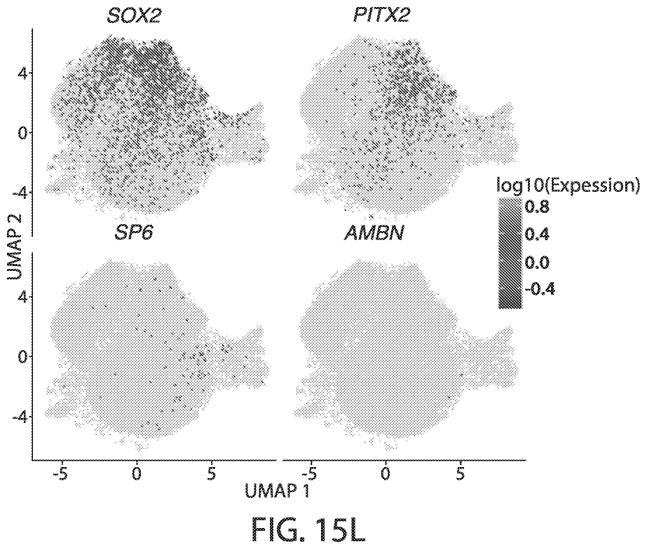

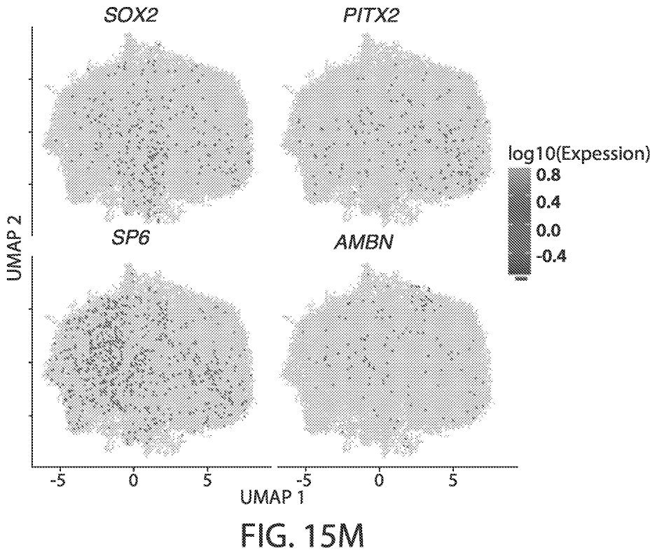

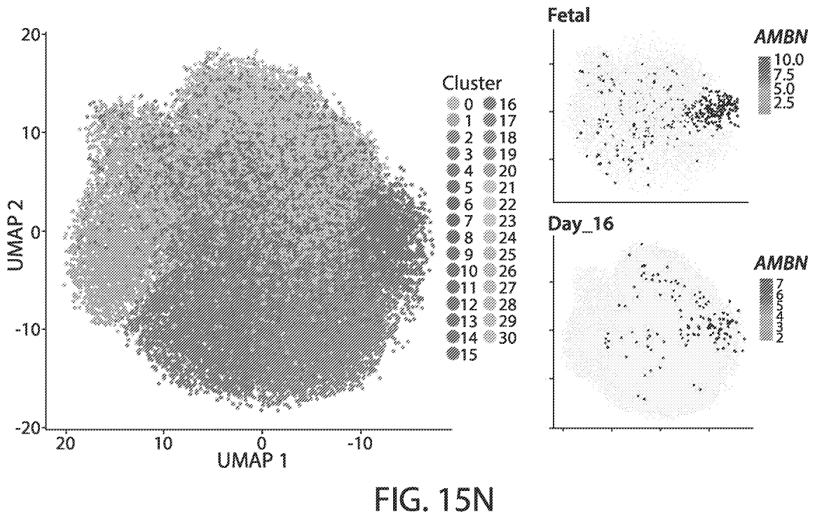

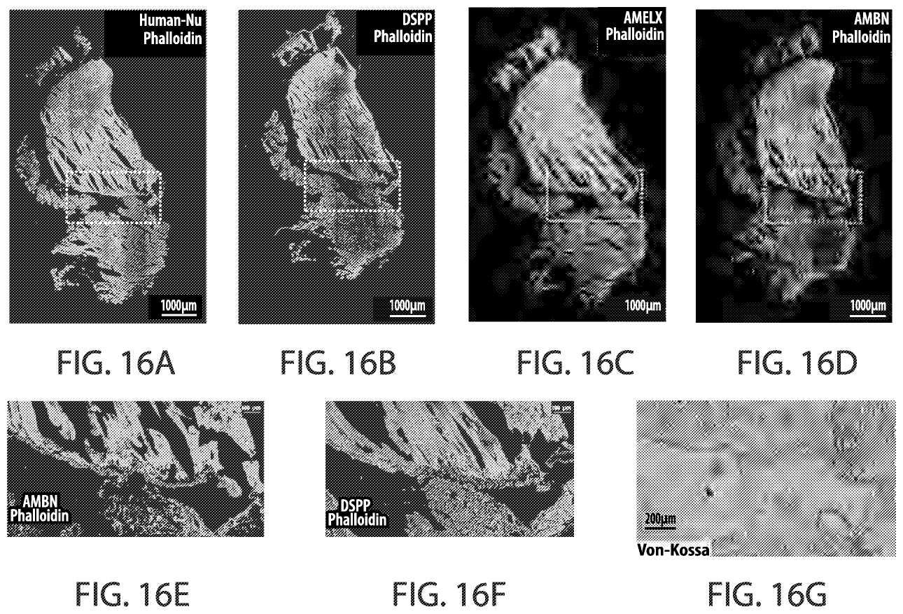

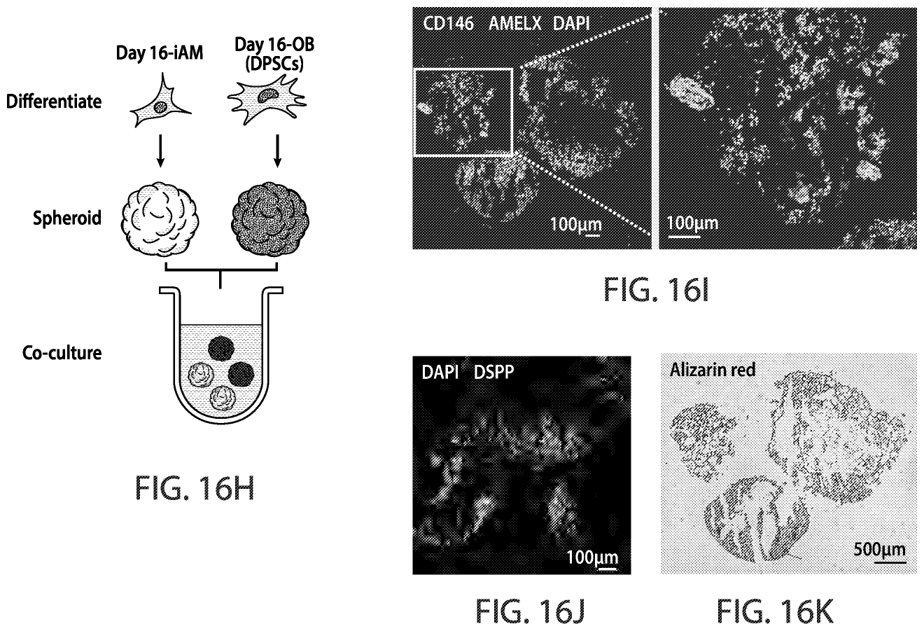

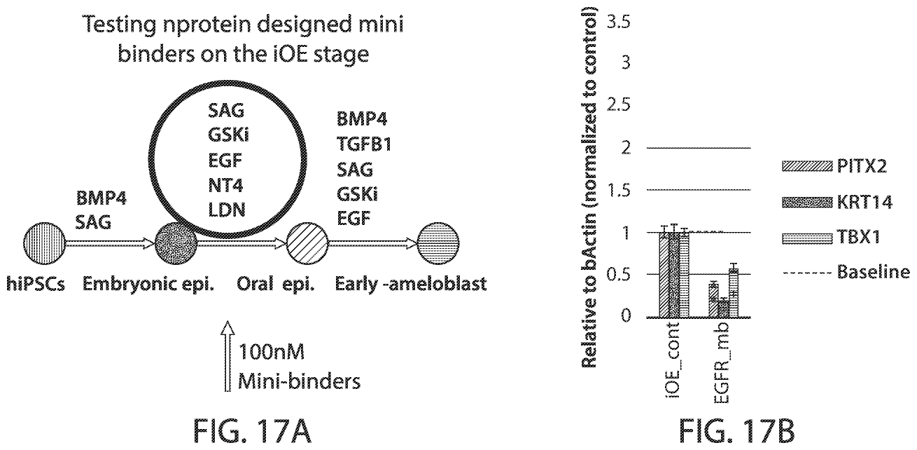

FIGS. 2J-2L: (FIG. 2J) UMAP graph of subclustered molar and incisor tooth germ type dental mesenchyme derived cells from the total dataset identified 6 transcriptionally unique clusters including dental papilla (DP), preodontoblast (POB), odontoblast (OB), subodontoblast (SOB), odontoblast (OB), dental ectomesenchyme (DEM), and dental follicle (DF). (FIG. 2K) A custom heatmap was generated to identify the marker genes specific to each cluster, the top associated GO-terms to characterize cluster function, and calculated age score per cluster. (FIG. 2L) Pseudotime trajectory analysis for dental mesenchyme derived cells suggest two progenitors DP and DEM, that give rise to differentiated OB.

FIGS. 2M-2N: (FIG. 2M) Real-time density plots indicate migration of cells from early progenitor populations DEM and DP at 9-16 gw to differentiated DF, POB, SOB and OB at later development 17-22 gw. (FIG. 2N) Simplified differentiation trajectory tree illustrating a common PRRX1+ progenitor gives rise to both DP and DEM. In the OB lineage, DP gives rise to POB, followed by OB, with a suggested SOB transitioning through POB-like state before giving rise to OB; and DF lineage, of DEM giving rise to DF.

FIGS. 2O-2R: (FIG. 2O) RNAScope HiPlex in situ hybridization image and inset including PRRX1, SOX5, FGF10, SALL1, IGFBP5, and KRT5 probes and DAPI at 80d. (FIG. 2P) RNAScope map for marker combinations corresponding to individual dental mesenchyme clusters for DEM (PRRX1+), DP (SOX5+FGF10+SALL1+) and DF (IGFBP5+) at 80d (arrows indicate DF within the dental pulp) with individual cell types and stage matched replicates mapped in FIG. 11. (FIG. 2Q) RNAScope HiPlex in situ hybridization image and inset including DSPP, IGFBP5, SALL1, FBN2, and KRT5 probes with DAPI nuclear stain at 117d. (FIG. 2R) RNAScope map for marker combinations corresponding to individual dental mesenchyme clusters for OB (DSPP+), SOB (IGFBP5+SALL1+), POB (FBN2+SALL1+), and DF (IGFBP5+) at 117d (SOB beneath OB at incisal edge and intermingled with POB) with individual cell types and stage matched replicates mapped in FIG. 11.

FIGS. 2S-2T: FIG. 2S shows a diagram of the developing dental mesenchyme derived cell types of the human tooth germ. At cap stage (12-13 gw) the dental pulp consists of DP cells with DEM with sparse DF within; DF surrounds the developing toothgerm. (FIG. 2T) By bell stage (17-22 gw), the dental pulp consists of OB at the incisal edge, SOB and POB with small contributions of the DEM and DP.

FIGS. 3A-3B: (FIG. 3A) UMAP graph of subclustered molar and incisor tooth germ type dental epithelium derived cells from the total dataset identified 13 transcriptionally unique clusters including the oral epithelium (OE), dental epithelium progenitors (DE-prog), initiation/enamel knot (IK/EK), enamel epithelium (OEE), stratum intermedium (SI), stellate reticulum (SR), pre-ameloblasts (PA) and ameloblasts (Am). (FIG. 3B) A custom heatmap was generated to identify the marker genes specific to each cluster, the top associated GO-terms to characterize cluster function, and calculated Age Score per cluster.

FIGS. 3C-3D: (FIG. 3C) UMAP graph of subclustered molar and incisor toothgerm type dental epithelium derived cells from the total dataset identified 13 transcriptionally unique clusters including the oral epithelium (OE), dental epithelium progenitors (DE), enamel knot (EK), outer enamel epithelium (OEE), inner enamel epithelium (IEE), cervical loop (CL), inner stratum intermedium (SII), outer stratum intermedium (SIO), inner stellate reticulum (SR), inner stellate reticulum (SRI), pre-ameloblasts (PA), early ameloblasts s(eAM) and ameloblasts (sAM). (FIG. 3D) A custom heatmap was generated to identify the marker genes specific to each cluster, the top associated GO-terms to characterize cluster function, and calculated Age Score per cluster.

FIGS. 3E-3I: (FIG. 3E) Pseudotime trajectory analysis for incisor dental epithelium derived cells indicates that the OE directly gives rise to four lineages including the OEE, SI, SR and EK. (FIG. 3F) Simplified differentiation trajectory tree illustrating the separate lineages originating from the DE, including the main Am lineage, of OEE, which gives rise to PA-1 and PA-2, which then gives rise to Am-1 and Am-2; and support cell trajectories (grey). Comparison of the incisor and molar tooth germ types proportions of cell type populations (FIG. 3G), timepoint at which Am first appear (FIG. 3H), and developmental scores calculated from expression profile of OE and Am markers to determine differentiation state (FIG. 3I).

FIG. 3J shows a diagram of the developing dental epithelium derived cell types of the incisor tooth germ. At 9-11 gestational weeks, the IK within the OE is present on one side of the dental placode, with the core cells of the bud structure consisting of DE. By 12-13 gestational weeks the DE has given rise to the signaling center EK and SR-1. By 14-16 gestational weeks, OEE present at the periphery of the tooth organ have given rise to PA-1 and PA-2; SI-1 and SI-2 lie near PA-1 and PA-2, respectively. AT 17-19 gestational weeks PA-2 differentiates into AM-1 and mature to AM-2, while SI-2 differentiates into SI-3, adjacent to AM-1 and AM-2.

FIG. 3K shows RNAScope HiPlex in situ hybridization image and inset for VWDE (high in IEE, SII, SRI), and FGF4 (high in EK) probes with DAPI nuclear stain at 80d RNAScope map of individual dental epithelium-derived clusters—EK, OEE, IEE, CL, SII, and SRI—present at 80d shown as determined by relative expression of markers as specified with individual cell types and stage matched replicates mapped in FIG. 16.

FIG. 3L shows a RNAScope HiPlex in situ hybridization image and inset for DSPP (high in eAM), ENAM (high in sAM), VWDE (high in SII, CL, PA), FBN2 (high in IEE, SII, CL) probes with DAPI nuclear stain at 117d.

FIG. 3M-3N shows a RNAScope map of individual dental epithelium derived clusters—IEE, PA, SII, SIO, OEE, CL, SRI, SRO, eAM, and sAM—present at 117d shown as determined by relative expression of markers as specified with stage matched replicates mapped in FIG. 11.

FIGS. 3O-3P: (FIG. 3O) A diagram of the developing dental epithelium derived cell types of a toothgerm at 12-13 gestational weeks. The OE is lining the oral cavity while DE is the stalk connecting OE to the enamel organ. DE has given rise to the signaling center EK and SRI. OEE present at the periphery of the enamel organ have given rise to CL, SII and IEE. (FIG. 3P) A diagram of the developing dental epithelium derived cell types of a toothgerm at 17-19 gestational weeks. SII give rise to SIO layer, and together represent the superficial layer above IEE, PA, eAM and sAM, while SRI and SRO represent the bulk of the cells inside the enamel organ.

FIGS. 4A-4P: Spatial Expression of Odontoblast and Ameloblast Markers Differs Markedly from Early to Late Toothgerm Development. (FIGS. 4A-4H) Odontoblast marker dentin sialophosphoprotein (DSPP) expression begins after the early bell stage in both the odontoblast (OB) and ameloblast (AM). (FIGS. 4I-4P) Ameloblast marker Ameloblastin (AMBN) expression begins after the early bell stage in both the OB and AM. Abbreviations: pre-odontoblast (POB), odontoblast (OB), pre-ameloblast (PA-1 and PA-2) ameloblast (AM-1 and AM-2), incisal edge (IE), cervical loop (CL), endothelial cells (EC). Scale bars: 50 μm.

FIG. 5A-5B show the most active signaling pathways involved in ameloblast differentiation were identified to be BMP, WNT, HH and FGF, with detailed description of workflow found in FIG. 15.

FIG. 5C-5D shows the sources of critical signaling ligands for the top three pathways involved for each developmental stage originate from both the dental epithelium and mesenchyme derived tissues (FIGS. 5C and 5D, left panel), with the thickness of the line indicating the number of ligand:receptor interactions, arrowheads indicating the cell possessing the receptor, and interactions of interest and between support cells. (FIGS. 5C and 5D, middle panel) Heatmaps for the top three pathways were generated by aggregating pathway ligand gene expression, which is then averaged per cluster. (FIGS. 5C and 5D, right panel) Diagrams illustrate the suggested ligand sources for each pathway at varying gestational weeks.

FIGS. 5E-5J: (FIG. 5E) Diagram for the proposed involvement of WNT pathway in activating the expression of SP6 which subsequently activate AMBN expression. Immunofluorescence staining of SP6 in 15 gw tooth germ (FIGS. 5F-5H) confirm the start of expression of SP6 in cytosol of IEE where we predict the initiation of WNT activity in (FIGS. 5C and 5D, right panel) at (OEE→IEE), in the region of CL. Endothelial cells (EC) are present within the developing dental pulp at the same stage. At 20 gw, SP6 is mainly localized to the nuclei coinciding with the onset of AMBN expression in differentiated AM at the tip of the toothgerm (FIGS. 5I and 5J). Scale bars: 50 μm.

FIGS. 6A-6C: (FIG. 6A) Schematic of the 16-day differentiation protocol produced, which targets the identified signalling pathways utilizing growth factors and small molecules to transition through the ameloblast developmental trajectory. Cells at Day 10 of differentiation show upregulated expression of oral epithelium markers PITX2 and KRT14 as assessed by QRT-PCR (FIG. 6B.1), while cells at Day 16 of differentiation show upregulation of ameloblast markers SP6 and AMBN as assessed by bulk RNA-seq (FIG. 6B.2) compared to undifferentiated HiPSC control. Each study was performed in triplicate, with error bars representing SEM. (FIG. 6C) 3D reconstruction via Imaris illustrates cell at Day 16 of differentiation positively express AMBN with cell membrane identified by ZO1, with quantification analysis finding approximately 25% of cells positive for AMBN expression. Each study was performed in triplicate, with error bars representing SEM. #D.1 and 3D.2 are different reconstructed region from original images shown in FIG. 11H.

FIGS. 6D-6E: (FIG. 6D) Projection of in vivo dental epithelium derived cell types (FIG. 3A) with Day 16 identified clusters suggests 67% of Day 16 cells share gene expression pattern of PA and OEE (FIG. 6D.1) and LIGER joint clustering analysis suggests 50% of Day 16 cells share gene expression pattern of Am and PA and OEE (D.2). (FIG. 6E) Label transfer of projection and integration identified cell types on induced ameloblast (iAM) Day 16 differentiation UMAP graph.

FIGS. 7A-7E: (FIG. 7A) Schematic of the 16-day differentiation protocol produced, which targets the identified signaling pathways utilizing growth factors and small molecules to transition through the ameloblast developmental trajectory. Cells at Day 10 of differentiation show upregulated expression of oral epithelium markers PITX2 and KRT14 as assessed by QRT-PCR (FIG. 7B.1), while cells at Day 16 of differentiation show upregulation of ameloblast markers SP6 and AMBN as assessed by bulk RNA-seq (FIG. 7B.2) compared to undifferentiated hiPSC control. Each study was performed in triplicate, with error bars representing SEM. (FIG. 7C) The efficiency of each pathway activated in the differentiation from day 10 to day 16 were assessed by removing each agonist one at a time or adding FGFR2 mini binder to inhibit the FGFR2 pathway. AMBN expression was assessed in QRT-PCR, and each condition was performed in triplicate. Significance was determined by unpaired Student's t-test; ***p<0.001; ****p<0.0001; Graph error bars are the means±SEM. (FIG. 7D) Projection of in vivo dental epithelium derived cell types (FIG. 3C) with Day 16 identified clusters suggests 60% of Day 16 cells share gene expression pattern of PA and eAM (FIG. 7D) and LIGER joint clustering analysis suggests 47% of Day 16 cells share gene expression pattern of AM and PA (FIG. 7E), which allowed the labels to be transfer and annotate the cell types identity on iAM Day 16 differentiation UMAP graph.

FIGS. 8A-8E: (FIG. 8A) Schematic of the mouse in vivo experiments describing the steps for injecting day 16-iAM subcutaneously into the left legs muscle of the adult SCID mice. The adult SCID mice at 2-month-old were dissected at the site of injection to perform further analysis to locate the cells such as immunofluorescence staining for human nuclear antigen in (FIG. 8B), KRT14 (FIG. 8C), AMELX (FIG. 8D) and Alizarin red staining in (FIG. 8E) showing mineralization.

FIG. 8F-8I: (FIG. 8F) Schematic of iAM organoids formation while cultured in suspension in ultra-low attachment plate. The formed iAM organoids express SP6 in the nuclei and secrete AMBN (FIG. 8H), and DSPP toward the apical side indicated by ZO1 (FIG. 8I). (FIG. 8G) A diagram simplifying the iAM organoid polarized structure toward a central lumen marked by ZO1, and the secretory vesicles of DSPP and AMBN.

FIGS. 8J-8N: (FIG. 8J) Schematic for the coculture experiment between DPSCs as monolayer and iAM embedded in Matrigel above it. Calcein, which is a fluorescent dye that binds to calcium, was added to the media containing iAM base media and odontogenic media in 1:1 ratio. (FIG. 8K) 3D reconstructed image from z-stacked confocal images captured from the coculture experiment plate. The cocultured organoid show association with Calcein, as well as expression of ENAM at the center after 7 days (FIG. 8L), and after 14 days the organoids close to CD146-expressing DPSC/OB, started to revert polarity towards DPSCs/OB while expressing AMELX (FIG. 8M) as simplified in the diagram in (FIG. 8N).

FIGS. 9A-9C: (FIG. 9A) Depending on the tissue size at a given age, between 3 and 50 of each tissue sample were collected, pooled into 12 samples, and sent for sci-RNA-seq. (FIG. 9B) At 9-11 w, dissecting individual toothgerms or salivary glands in the bud stage was not feasible due to the large number of cells required to perform sci-RNA-seq protocol. Instead, jaws were separated into two segments of posterior jaw, containing jaw tissue distal of the canines (FIG. 9B), and one segment of anterior jaw spanning from canine tooth to canine tooth region (FIG. 9B). At 12 weeks gestation and beyond, individual toothgerms and submandibular salivary glands could be identified and isolated to be sequenced separately (FIG. 9C). Sequenced data was clustered, and the resulting plot revealed that each major tissue type occupied a specific region of the plot, with some shared support tissues localized in the center.

FIGS. 9D-9G: (FIG. 9D) Clusters were identifiable by expression of known markers for each tissue type in heatmap (FIG. 9E). QC table for all sequenced data (FIG. 9F). Immunofluorescence staining of developing molar tooth germ with anti-Krt5 that specifically mark the dental epithelial morphology. Counterstained with the nuclear staining DAPI (FIG. 9G).

FIGS. 10A-10E: Gene density plots of putative marker genes for dental ectomesenchyme (DEM) (FIG. 10A), preodontoblast (POB) (FIG. 10B), subodontoblast progenitor (SOBP) (FIG. 10C), subodontoblast (SOB) (FIG. 10D), and odontoblast (OB) (FIG. 10E) in the incisor and merged incisor and molar datasets show similar gene expression profiles.

FIGS. 10F-10G: Heatmaps of putative marker genes show similar cell type specific expression patterns in the merged (FIG. 10F) and incisor only (FIG. 10G) datasets.

FIGS. 10H-10J: (FIG. 10H) Heatmap of expression over time of dental follicle marker IGFBP5 and subodontoblast markers SALL1 (FIG. 10I). Gene plot of shared DP and DEM progenitor marker PRRX1 (FIG. 10J). Cell cycle scoring of dental mesenchyme derived cell types.

FIGS. 10K-10M: (FIG. 10K). Mappings for dental mesenchyme-derived cell types at 80d replicate (FIG. 10L) and 117d replicate (FIG. 10M) identified by analysis of RNAScope images which show mapping for SOB, DF, DEM, OB, and POB cell types.

FIG. 10N shows real time density plots illustrate delayed migration of cells from progenitor DEM and SOBP to mature OB in the molar compared to the incisor.

FIGS. 11A-11B: Expression of known markers at stages of dental epithelial lineage align with previously identified markers in each tissue and appear at expected developmental timepoints (FIG. 11A). Density of cells plotted by age demonstrates that between incisors (top) and molars (bottom), clusters enriched with more cells at a given timepoint do not differ (FIG. 11B).

FIG. 11C-11D: (FIG. 11C) Expression of known markers at stages of dental epithelial lineage align with previously identified markers in each tissue and appear at expected developmental timepoints (FIG. 11C). Density of cells plotted by age demonstrates that clusters enriched with more cells at a given timepoint (FIG. 11D).

FIG. 11E: Gene plots and mean expression per cluster summary plots in UMAP space (FIG. 11E) generated for the markers used to infer the logic table which is used in the RNAScope mapping. The threshold expression per cluster was set to 25% of maximum expression per gene. Light clusters considered as high expressing, dark clusters as low expressing, and gray as low or no expression.

FIGS. 11F-11G: Mappings for dental epithelium-derived cell types in a stage matched (80d) replicate sample (FIG. 11F) identified by analysis of RNAScope images showing the mappings for EK, OEE, IEE, CL, SII, and SRI cell types in composite, and at (117d) replicate sample (FIG. 11G) mappings for IEE, PA, SII, SIO, OEE, CL, SRI, SRO, eAM, and sAM cell types at 117d shown in composite.

FIGS. 11H-11I: Using LIGER joint clustering to integrate the in vivo dataset of human dental epithelial lineage (from FIG. 3C) with previously published sci-RNA-seq data of mouse incisors yielded 6 overlapped clusters (FIG. 11H). Further analysis was able to match the known labels from the in vivo dataset of dental epithelial lineage (right, FIG. 11I) to the joint clusters produced by LIGER (middle, FIG. 11I), then to the clusters from of mouse incisors (left; FIG. 11I).

FIGS. 12A-12W: Ameloblast markers Amelogenin (AMELX) expression begins after the early bell stage in AM (FIGS. 12A-12J). Early ameloblast marker SP6 expression begins in cytosol of PA at the early bell stage, before shifting expression transiently to AM at late bell stage. Note the clear demarcation of PA in the cervical loop (CL) (FIGS. 12K-12S). Endothelial cells (EC) are present within the developing dental pulp at the early bell stage (FIGS. 12K-12Q). Expression of SP6 can be noted in odontoblasts as well at late bell stage (FIG. 12T). The ameloblast marker ameloblastin (AMBN), and the odontoblast marker (DSPP) cannot be detected at the cervical loops of either stage examined as the differentiation is only localized at the incisal edge at those stages (FIGS. 12U-12W). Abbreviations: pre-odontoblast (POB), odontoblast (OB), pre-ameloblast (PA-1 and PA-2) ameloblast (AM-1 and AM-2), incisal edge (IE), cervical loop (CL), endothelial cells (EC). Scale bars: 50 μm

FIGS. 13A-13Y: Ameloblast markers amelogenin (AMELX) and ameloblastin expression begins in the ameloblast after the early bell stage (FIGS. 13A-13J, FIGS. 13K-13R). Similarly, odontoblast marker dentin sialo phosphoprotein (DSPP) begins in the odontoblast after the early bell stage (FIGS. 13S-13Y). Heatmaps of expression over time of AMELX (E), AMBN (O), and DSPP (W). AMELX, AMBN and DSPP show mirrored expression patterns in ameloblasts and odontoblasts at late bell stage (H,Q,Y). Abbreviations: preodontoblast (POB), odontoblast (OB), preameloblast (PA) ameloblast (AM), incisal edge (IE), cervical loop (CL). Scale bars: 50 μm.

FIG. 14A-14C: The inventors developed a combined computations workflow to identify critical pathways at different developmental steps, represented by the schematic in (FIG. 14A), yielding a ranking and contribution to ameloblast differentiation for several major pathways (FIG. 14B) over several developmental stages. Using talklr ligand-receptor analysis, the inventors identified the sources of the outgoing signals at early stages of ameloblast development (FIG. 14C). The top three pathways per stage are indicated in ligand:receptor interaction graphs (FIG. 14C, left panel). The thickness of the arrows indicates the number of unique possible interactions between clusters. The arrowheads indicate the receiver cells that express the receptors. Arrows highlighting the interactions received by the cells of interest at each stage. Heatmaps generated by first aggregating ligands gene expression for each cell, and then the average values are calculated per cluster (FIG. 14C, middle panel). Analyses demonstrate that at the placode stage, BMP and Activin signals come from the initiation knot and mesenchyme to the epithelium while at the bud stage, the initiation know and the oral epithelium itself secrete WNT signals. In the early cap stages, WNT signals come from the dental epithelium while the mesenchyme secretes neurotrophin signals toward the dental epithelium. At the late cap stage, following the development of the stellate reticulum (SR-1), WNT signals switch to non-canonical WNT signaling from the SR-1 to the enamel knot (FIG. 14C, right panel).

FIG. 14D-14E: The inventors developed a combined computations workflow to identify critical pathways at different developmental steps, represented by the schematic in (FIG. 14A), yielding a ranking and contribution to ameloblast differentiation for several major pathways (FIG. 14D) over several developmental stages. Using talklr ligand-receptor analysis, the sources of the outgoing signals were identified at early stages of ameloblast development (FIG. 14E). The top three pathways per stage are indicated in ligand:receptor interaction graphs (FIG. 14E, left panel). The thickness of the arrows indicates the number of unique possible interactions between clusters. The arrowheads indicate the receiver cells that express the receptors. Arrows highlighting the interactions received by the cells of interest at each stage. Heatmaps generated by first aggregating ligands gene expression for each cell, and then the average values are calculated per cluster (FIG. 14E, middle panel). Analyses demonstrate that at the placode stage, BMP and Activin signals come from the mesenchyme (DEM) to the epithelium while at the bud stage the oral and dental epithelium itself secrete WNT signals. In the early cap stages, WNT signals come from the dental epithelium while the mesenchyme secretes FGF and BMP signals toward the dental epithelium. At the late cap stage, following the development of the inner stellate reticulum (SRI), WNT signals switch to non-canonical WNT signaling from the SRI to the enamel knot (FIG. 14E, right panel).

FIG. 15A-15B: (FIG. 15A) Brightfield images of hiPSCs, day 10 of in vitro differentiation, and isolated fetal oral epithelium after culturing for seven days (FIG. 15A) show that oral epithelium differentiated from iPSCs exhibit the same morphological characteristics as culture human oral epithelium. Quantitative PCR (FIG. 15B) showed that compared to undifferentiated hiPSCs, differentiated oral epithelium exhibited elevated levels of known oral epithelium markers concomitant with a significant decrease in known pluripotency marker OCT-4. Additionally, the neuroepithelial marker NESTIN, and the early mesodermal marker TBXT (BRACHYURY) are relatively unchanged at day 10 of the differentiation, indicative of a relatively lineage-specific differentiation.

FIGS. 15C-15F: Successful further differentiation of oral epithelium into ameloblasts was demonstrated by immunofluorescence staining of day 16 (FIG. 15C), showing AMBN expression, and the membrane marker ZO1. Selected regions were 3D reconstructed using Imaris software and shown in FIG. 6C. Day 10 (FIG. 15D) and Day 16 (FIG. 15E) samples were sequenced with sci-RNA-seq. Cells were clustered and analyzed to identify clusters with similar gene expression patterns to known cell types in fetal development. Gene expression density plots for known markers of different phases of ameloblast development show continuity between day 10 and day 16, with early markers SOX2 and PITX2 being predominantly expressed by day 10 (FIG. 15F) and shifting toward ameloblast-specific markers AMBN and SP6 in day 16 (FIG. 15G). LIGER joint clustering analysis of Day 16 differentiation cells and the in vivo human fetal dental epithelium (FIG. 3A) derived cells suggests the colocalization of AMBN expressing cells from in vivo and in vitro in cluster 6 (FIG. 15H).

FIGS. 15I-N: Successful further differentiation of oral epithelium into ameloblasts was demonstrated by immunofluorescence staining of day 16 (FIG. 15I), showing AMBN expression, and the membrane marker ZO1, with quantification analysis finding approximately 25% of cells positive for AMBN expression. Each study was performed in triplicate, with error bars representing ±SEM. Significance was determined by unpaired Student's t-test; ***p<0.001; ****p<0.0001. Day 10 (FIG. 15J) and Day 16 (FIG. 15K) samples were sequenced with sci-RNA-seq. Cells were clustered and analyzed to identify clusters with similar gene expression patterns to known cell types in fetal development. Gene expression density plots for known markers of different phases of ameloblast development show continuity between day 10 and day 16, with early markers SOX2 and PITX2 being predominantly expressed by day 10 (FIG. 15L) and shifting toward ameloblast-specific markers AMBN and SP6 in day 16 (FIG. 15M). LIGER joint clustering analysis of Day 16 differentiation cells and the in vivo human fetal dental epithelium (FIG. 3A) derived cells suggests the colocalization of AMBN expressing cells from in vivo and in vitro in cluster 6 (FIG. 15N).

FIGS. 16A-16K: (FIGS. 16A-16D) Immunofluorescence staining shows the entire surface area of the stained muscle sections in FIGS. 8B-8E. The dotted boxes indicate the area of interest magnified in main FIG. 8 and in (FIGS. 16E-16G) for AMBN and DSPP. Von-Kossa staining for calcification was performed in a subsequent section in (FIG. 16G) showing black/brown staining localized to the injected cell region. (FIG. 16H) A schematic for the coculture experiment between iAM organoids and DPSC organoids in suspension culture where each cell type was formed in separate wells and then combined for 14 days in iAM base media. Organoids were snap frozen, cry-sectioned, and prepared for immunofluorescence. Expression of AMELX was noted in iAM organoids (FIG. 16I) and CD146 and DSPP in DPSC/OB organoids (FIG. 16J). Alizarin red staining in (FIG. 16K) indicates the classification is positive in both organoid types, particularly DSPC/OB, which shows more calcifications.

FIG. 17A-17B examines designed protein mini binders can be used to study and enhance iOE differentiation. FIG. 17A shows simplified schematic of ameloblast in vitro differentiation indicating the timing where the designed mini-binders were tested specifically at the oral epithelium stage between day 8 and day 9. FIG. 17B shows the samples from oral epithelium stage were collected and analyzed for the expression of the oral epithelium markers, PITX2, KRT14, and TBX1 which were assessed by QRT-PCR, and each condition was performed in duplicate. Significance was determined by unpaired Student's t-test; *p<0.05; Graph error bars are the means±SEM.

### DETAILED DESCRIPTION

Provided herein are methods for producing ameloblasts in vitro from induced pluripotent stem cells (iPSCs). Also provided herein are compositions comprising such ameloblasts that can be administered to subjects having a disease or disorder associated with demineralization of dental enamel, weakening of enamel, or impaired production of enamel, such as amelogenesis imperfecta.

### Definitions

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

As used herein, the term “contacting” when used in reference to a cell, encompasses both introducing an agent, growth factor, surface, etc. to the cell in a manner that permits physical contact of the cell with the agent, growth factor, surface, etc., and introducing an element, such as a genetic construct or vector, that permits the expression of an agent, such as an miRNA, polypeptide, or other expression product in the cell. It should be understood that a cell genetically modified to express an agent, is “contacted” with the agent, as are the cell's progeny that express the agent.

The terms “stem cell” or “undifferentiated cell” as used herein, refer to a cell in an undifferentiated or partially differentiated state that has the property of self-renewal and has the developmental potential to differentiate into multiple cell types, without a specific implied meaning regarding developmental potential (i.e., totipotent, pluripotent, multipotent, etc.). A stem cell is capable of proliferation and giving rise to more such stem cells while maintaining its developmental potential. In theory, self-renewal can occur by either of two major mechanisms. Stem cells can divide asymmetrically, which is known as obligatory asymmetrical differentiation, with one daughter cell retaining the developmental potential of the parent stem cell and the other daughter cell expressing some distinct other specific function, phenotype and/or developmental potential from the parent cell. The daughter cells themselves can be induced to proliferate and produce progeny that subsequently differentiate into one or more mature cell types, while also potentially retaining one or more cells with parental developmental potential. A differentiated cell may derive from a multipotent cell, which itself is derived from a multipotent cell, and so on. While each of these multipotent cells can be considered stem cells, the range of cell types each such stem cell can give rise to, i.e., their developmental potential, can vary considerably. Alternatively, some of the stem cells in a population can divide symmetrically into two stem cells, known as stochastic differentiation, thus maintaining some stem cells in the population as a whole, while other cells in the population give rise to differentiated progeny only. Accordingly, the term “stem cell” refers to any subset of cells that have the developmental potential, under particular circumstances, to differentiate to a more specialized or differentiated phenotype, and which retain the capacity, under certain circumstances, to proliferate without substantially differentiating. In some embodiments, the term “stem cell” refers generally to a naturally occurring parent cell whose descendants (progeny cells) specialize, often in different directions, by differentiation, e.g., by acquiring completely individual characters, as occurs in progressive diversification of embryonic cells and tissues. Some differentiated cells also have the capacity to give rise to cells of greater developmental potential. Such capacity may be natural or may be induced artificially upon treatment with various factors. Cells that begin as stem cells might proceed toward a differentiated phenotype, but then can be induced to “reverse” and re-express the stem cell phenotype, a term often referred to as “dedifferentiation” or “reprogramming” or “retrodifferentiation” by persons of ordinary skill in the art, and as used herein.

Exemplary stem cells include embryonic stem cells, adult stem cells, pluripotent stem cells, bone marrow stem cells, mesenchymal stem cells, hematopoietic stem cells, and the like. Descriptions of stem cells, including methods for isolating and culturing them, may be found in, among other places, Embryonic Stem Cells, Methods and Protocols, Turksen, ed., Humana Press, 2002; Weisman et al., Annu. Rev. Cell. Dev. Biol. 17:387 403; Pittinger et al., Science, 284:143 47, 1999; Animal Cell Culture, Masters, ed., Oxford University Press, 2000; Jackson et al., PNAS 96(25):14482 86, 1999; Zuk et al., Tissue Engineering, 7:211 228, 2001 (“Zuk et al.”); Atala et al., particularly Chapters 33 41; and U.S. Pat. Nos. 5,559,022, 5,672,346 and 5,827,735.

In the context of cell ontogeny, the term “differentiate”, or “differentiating” is a relative term that indicates a “differentiated cell” is a cell that has progressed further down a developmental pathway than its precursor cell. Thus in some embodiments, a stem cell as the term is defined herein, can differentiate to lineage-restricted precursor cells, which in turn can differentiate into other types of precursor cells further down the pathway, and then to an end-stage differentiated cell, which plays a characteristic role in a certain tissue type, and may or may not retain the capacity to proliferate further. The differentiation status of a cell is generally determined by one or more of characteristic gene or marker expression pattern, metabolic activit(ies), and morphology.

The term “pluripotent” as used herein refers to a cell with the capacity, under different conditions, to differentiate to cell types characteristic of all three germ cell layers (endoderm, mesoderm and ectoderm). Pluripotent cells are characterized primarily by their ability to differentiate to all three germ layers, using, for example, a nude mouse and teratoma formation assay. Pluripotency is also evidenced by the expression of embryonic stem (ES) cell markers, although the preferred test for pluripotency is the demonstration of the capacity to differentiate into cells of each of the three germ layers.

The term “reprogramming” as used herein refers to a process that alters or reverses the differentiation state of a differentiated cell (e.g., a somatic cell). Stated another way, reprogramming refers to a process of driving the differentiation of a cell backwards to a more undifferentiated or more primitive type of cell. The cell to be reprogrammed can be either partially or terminally differentiated prior to reprogramming. In some embodiments, reprogramming encompasses complete reversion of the differentiation state of a differentiated cell (e.g., a somatic cell) to a pluripotent state. In some embodiments, reprogramming also encompasses partial reversion of the differentiation state of a differentiated cell (e.g., a somatic cell) to a multipotent state. In some embodiments, reprogramming encompasses complete or partial reversion of the differentiation state of a differentiated cell (e.g., a somatic cell) to an undifferentiated cell. Reprogramming also encompasses partial reversion of the differentiation state of a somatic cell to a state that renders the cell more susceptible to complete reprogramming to a pluripotent state when subjected to additional manipulations.

Reprogramming involves alteration, e.g., reversal, of at least some of the heritable patterns of nucleic acid modification (e.g., methylation), chromatin condensation, epigenetic changes, genomic imprinting, etc., that occur during cellular differentiation as a zygote develops into an adult.

As used herein, the terms “iPSC” and “induced pluripotent stem cell” are used interchangeably and refer to a pluripotent cell artificially derived from a differentiated somatic cell. iPSC cells are capable of self-renewal and differentiation into cell fate-committed stem cells, including cells associated with tooth development, as well as various types of mature cells. As used herein, the term hiPSC refers to iPSCs derived from human somatic cells.

The term “derived from,” used in reference to a stem cell means the stem cell was generated by reprogramming of a differentiated cell to a stem cell phenotype. The term “derived from,” used in reference to a differentiated cell means the cell is the result of differentiation, e.g., in vitro differentiation, of a stem cell.

A “marker” as used herein refers to one or more characteristics that contribute to or are associated with the phenotype of a given cell. Markers can be used for selection of cells comprising characteristics of interest. Markers will vary with specific cells, and can be characteristics, whether morphological, functional or biochemical, that are particular to a cell or tissue type, or to a disease state or phenotype and include both extracellular and intracellular molecules, e.g., proteins, RNAs, glycosylation patterns, etc. expressed or exhibited by the cell or tissue. In some embodiments, such markers are proteins, including, but not limited to proteins that possess an epitope for an antibody or other binding molecule available in the art. Other markers can include peptides, lipids, polysaccharides, nucleic acids and steroids, among others. Examples of morphological characteristics or traits include, but are not limited to, shape, size, and nuclear to cytoplasmic ratio. Examples of functional characteristics or traits include, but are not limited to, the ability to produce dental enamel. Markers can be detected by any appropriate method available to one of skill in the art.

The terms “decrease,” “reduced,” “reduction,” “decrease,” or “inhibit” as used herein generally refer to a decrease by a statistically significant amount. However, for avoidance of doubt, “reduced”, “reduction” or “decrease” or “inhibit” mean a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased,” “increase,” or “enhance,” or “activate” as used herein generally refer to an increase by a statically significant amount. However, for the avoidance of doubt, the terms “increased”, “increase” or “enhance” or “activate” mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase or more, relative to a reference level.

As used herein, the term “modulates” refers to an effect including increasing or decreasing a given parameter as those terms are defined herein.

As used herein, the term “organoid” refers to a 3D multicellular tissue culture construct comprising cells differentiated in vitro from stem cells, including, but not limited to iPS cells. Organoids can, but need not necessarily include a plurality of different cell types. In one embodiment, an organoid comprising ameloblasts, e.g., differentiated as described herein, comprises a spherical arrangement of ameloblasts and extracellular matrix in which the cells are arranged and oriented so as to secrete ameloblastin into a lumen of the organoid.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth. It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

The disclosure described herein, in a preferred embodiment, does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

### Preparation of Stem Cell-Derived Ameloblasts

Provided herein are methods for generating ameloblasts from induced pluripotent stem cells. Also described herein are methods of producing stem cell-derived ameloblasts beginning with somatic cells derived from a subject, patient or donor, including a subject having a given disease or disorder that affects enamel production or enamel maintenance. The somatic cells are reprogrammed to induced pluripotent stem cells (iPS cells, iPSCs), which are then differentiated to ameloblasts or organoids comprising ameloblasts. Thus, described herein are methods of reprogramming somatic cells to iPS cells, and also described herein are methods of differentiating iPS cells to stem cell-derived ameloblasts.

Sources of Somatic Cells for Reprogramming into iPS Cells:

Stem cell derived-ameloblasts are produced from a donor cell induced to a pluripotent stem cell phenotype that is then differentiated along the ameloblast lineage. Without limitations, iPS cells can be produced from any animal in addition to humans. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. The cell donor, patient, or subject can include any of the subset of the foregoing as appropriate for a given use. In certain embodiments of the aspects described herein, the subject is a mammal, e.g., a primate, e.g., a human.

iPS cells can also be produced from donor stem cells (e.g., human donor stem cells that can be autologous or allogeneic). Preference is given to cells that are easily obtained and available in sufficient numbers for efficient isolation. Exemplary stem cells include adult stem cells, bone marrow stem cells, placental stem cells, lymphoid stem cells, mesenchymal stem cells, hematopoietic stem cells, and the like.

More often, iPS cells will be generated from differentiated donor cells (e.g., human cells) including nucleated somatic cells including, but not limited to fibroblasts, stromal cells, muscle cells or cells of any of a wide number of tissues in the adult organism. Donor cells can be obtained from the subject by a skin biopsy, urine sample, or by drawing blood, among other methods.

### Reprogramming of Somatic Cells to iPS Cells:

Methods of reprogramming differentiated cells to iPS cells are well known in the art and generally involve forced expression of Oct3 or Oct4, Sox2, Klf4, and c-Myc in the cells, although numerous variations are known in the art. hiPSCs are cultured, expanded and passaged according to the methods described herein or other conditions favorable to cell viability and maintenance of the undifferentiated, pluripotent phenotype. hiPSCs are maintained, for example in hypoxic conditions (e.g., 37° C., 5% CO₂, 5% O₂).

Without limitations, iPS cells can also be generated using other methods, including, but not limited to non-viral methods, use of polycistronic vectors, mRNA species, miRNAs, and proteins, including methods described in, for example, International Patent Applications WO2010/019569, WO2009/149233, WO2009/093022, WO2010/022194, WO2009/101084, WO2008/038148, WO2010/059806, WO2010/057614, WO2010/056831, WO2010/050626, WO2010/033906, WO2009/126250, WO2009/143421, WO2009/140655, WO2009/133971, WO2009/101407, WO2009/091659, WO2009/086425, WO2009/079007, WO2009/058413, WO2009/032456, WO2009/032194, WO2008/103462, JP4411362, EP2128245, and U.S. Patent Applications US2004/0072343, US2009/0253203, US2010/0112693, US2010/07542, US2009/0246875, US2009/0203141, US2010/00625343, US2009/0269763, and US2010/059806, each of which are incorporated herein in their entirety by reference. While any tissue can provide source cells for generating iPS cells to differentiate into ameloblasts, it is contemplated that cells derived from dental gum tissue may have benefits, for example, epigenetic memory, that permit enhanced generation of mature ameloblasts when used in the methods described herein to generate iPS cell-derived ameloblasts.

In some embodiments, the differentiated cell can be an epithelial cell (e.g., an epithelial keratinocyte, such as an oral epithelial keratinocyte, or an epithelial cell of the skin). Oral epithelial cells can be harvested from any area of the oral cavity in which they reside (e.g., the palate). In any embodiment, the cell can be immortalized and/or genetically modified.

In some embodiments, the ameloblasts or ameloblast organoids described herein can be generated from enamel organ epithelial (EOE) cells or cell lines thereof. As noted, the cells can be immortalized, and they can in that instance be, for example, the immortalized EOE cell line generated by DenBesten et al., PABSo-E (DenBesten et al., Eur. J. Oral Sci., 107(4):276-81, 1999). Another useful cell type is the Epithelial Cell Rests of Malassez (ERM cells; see Shinmura et al., J. Cellular Physiol. 217:728-738, 2008). ERM cells can be obtained from periodontal ligament tissue by explant culture, sub-cultured with non-serum medium, and expanded on 3T3-J2 feeder cell layers. The ameloblasts or organoids thereof can also be generated from skin epithelial cells (see Liu et al., J. Tissue Eng. Regen. Med. 7:934-943, 2012).

### Cell Culture

The ameloblasts or the cells from which they are produced can be cultured in a conventional two-dimensional culture initially, where they grow primarily in monolayers, and then transferred to a 3D culture, or they may be placed directly into the 3D culture. For example, where the methods employ differentiated cells that are modified through genetic or recombinant methods or by de- and re-differentiation to produce ameloblastin, the cells can be placed initially into a two-dimensional culture that is not designed to support growth in three dimensions. When 3D growth is desired, a cell or cell type, including ameloblasts prepared as described herein can be mixed with a substrate such as a basement membrane matrix (e.g., BD Matrigel™ from BD Bioscience, San Jose Calif; catalog #356234; see Hughes et al., Proteomics 10(9):1886-1890, 2010), and grown in vitro using growth media (e.g., DMEM-high sucrose). Exemplary materials for 3D culture growth include, but are not limited to: BD Matrigel™ Basement Membrane Matrix, and growth-factor reduced Matrigel™. While the precise composition of the matrix can vary, it can include a mixture of extracellular matrix proteins and growth factors, and it can be derived from tumor cells (e.g., Engelbreth-Holm-Swarm (EHS) mouse sarcoma cells). The matrix can be one that, in the absence of other factors, maintains a pluripotent cell in its pluripotent state (i.e., it promotes self-renewal).

In three-dimensional cultures, the cells can become multilayered or clustered with the layered and/or clustered cell growth being guided by scaffolds. For example, 3D cultures can be grown in AggreWell™ 400 plates (Stemcel Technologies, Grenoble, France). The 3D cultures of ameloblasts or organoids can be grown in bioreactors to support spheroid growth and the development of an extracellular matrix. More generally, one can use 3D cultures based on or generated by extracellular matrices or scaffolds (e.g., employing hydrogels), modified surfaces, rotating bioreactors, microcarriers, magnetic levitation, hanging drop plates, and/or magnetic 3D bioprinting. In scaffold-free techniques, one can employ low adhesion plates and micropatterned surfaces.

Exemplary extracellular matrices include, but are not limited to Matrigel™ (e.g., a basement-membrane matrix extracted from Engelbreth-Holm-Swarm mouse sarcomas), matrices from other cell types and synthetic matrices (see eg., Aisenbrey, E. A., Murphy, W. L. Synthetic alternatives to Matrigel. Nat Rev Mater 5, 539-551 (2020) which is incorporated herein by reference in its entirety).

### Detection of Ameloblasts and Precursors Thereof

The production of ameloblasts can be confirmed by detecting one or more markers of ameloblasts or by detecting the presence of functions associated with ameloblasts, such as enamel production. In some embodiments, the production of ameloblasts can be confirmed by detecting the presence of one or more markers of ameloblast differentiation including, but not limited to, ameloblastin, amelogenin, tuftelin, enamelin, MMP-20 (matrix metalloproteinase-20), EMSP1 (enamel matrix seine proteinase 1), and cytokeratin 14.

Alternatively, one can detect the presence of ameloblasts by detecting enamel production or deposition of calcium when cells are cultured in the presence of a mineralizing solution (e.g., calcium 2.5 mM, phosphate 1.5 mM).

In some embodiments, cells in the oral epithelium stage can be detected by a relative increase in gene expression of PITX2, TBX1, & TP63 as markers, e.g., by at least 10% compared to cells in the prior stage (e.g., embryonic epithelium stage or iPSC). In some embodiments, the levels of PITX2, TBX1 and TP63 are increased by at least 20%, at least 30%, at least 40% at least 50%, at least 60%, at least 70%, at least 80%, at least 90%, at least 1-fold, at least 2-fold, at least 5-fold, at least 10-fold, at least 100 fold or more at the oral epithelium stage compared to an earlier stage in the method (e.g., embryonic epithelium stage or iPSC).

### Signaling Pathways

Sonic Hedgehog signaling pathway modulators: In the methods described herein, one or more Hedgehog (Hh) signaling pathway modulating agents can be used in steps of the method to induce the differentiation of an iPSC cell to an ameloblast. Hh proteins are key molecules for diverse tissue patterning processes in both invertebrates and vertebrates. For example, in Drosophila, Hh is crucial for the development of a segmented body plan and the patterning of imaginal tissues, whereas in vertebrates Sonic hedgehog (Shh) functions to pattern limb buds and promote cell fate specification, proliferation, and axon guidance in the central nervous system. Shh initiates signaling by binding the 12-pass transmembrane protein Patched1 (PTCH1). Upon Shh binding, the inhibition exerted by PTCH1 on the transmembrane protein Smoothened (Smo) is relieved, eliciting a signaling cascade which ultimately leads to Gli-mediated transcription.

In certain embodiments, an agent that induces signaling of the Sonic Hedgehog signaling pathway is used in the claimed method to produce ameloblasts. Agents that induce the SHH pathway include, but are not limited to, smoothened agonists (e.g., SAG, purmorphamine), inhibitors of Patched-1 (e.g., cyclopamine) or SHH ligands.

The methods described herein comprise contacting an iPSC cell with smoothened agonist (SAG; 3-chloro-N-[(1r,4r)-4-(methylamino)cyclohexyl]-N-[3-(pyridin-4-yl)benzyl]benzo[b]thiophene-2-carboxamide). Alternatively, in some embodiments the smoothened agonist comprises purmorphamine (PMN).

In some embodiments, the dosage range for SAG used, in part, to promote differentiation of iPSC to ameloblasts is between 200 nM and 10 μM, for example, between 200 nM and 1 μM, between 200 nM and 750 nM, between 200 nM and 500 nM, between 200 nM and 400 nM, between 200 nM and 300 nM, between 300 nM and 1 μM, between 400 nM and 1 μM, between 500 nM and 1 μM, between 500 nM and 1 μM, between 600 nM and 1 μM, between 700 nM and 1 μM, between 800 nM and 1 μM, between 900 nM and 1 μM, between 300 nM and 600 nM, between 200 nM and 500 nM, between 300 nM and 500 nM, between 350 nM and 450 nM, or any range therebetween.

BMP pathway modulation: In one embodiment, one or more BMP pathway agonists are used to promote a particular differentiation step of a pluripotent cell. In such embodiments, an agonist of BMP signaling can be a polypeptide or fragment thereof, or a small molecule agonist of a BMP receptor. In some embodiments, the BMP pathway that is induced comprises the BMP4 pathway. BMP4 is a protein involved in a signal transduction pathway inducing the differentiation of pluripotent stem cells into mesodermal cells. Thus, in some embodiments, the agent that induces the BMP4 pathway comprises BMP4, an active fragment thereof or a BMP4 agonist.

In some embodiments, the dosage range useful for BMP4 or a BMP4 agonist is between 50 and 900 pM, for example between 50 and 800 pM, between 50 and 700 pM, between 50 and 600 pM, between 50 and 500 pM, between 50 and 400 pM, between 50 and 300 pM, between 50 and 200 pM, between 50 and 100 pM, between 100 and 800 pM, between 150 and 800 pM, between 200 and 800 pM, between 300 and 800 pM, between 400 and 800 pM, between 500 and 800 pM, between 600 and 800 pM, between 700 and 800 pM, between 150 and 750 pM, between 150 and 400 pM, between 150 and 300 pM, between 200 and 500 pM, between 300 and 700 pM, or any dose therebetween.

BMP receptor pathway inhibition: In one embodiment, one or more BMP receptor pathway antagonists are used to promote a particular differentiation step of a pluripotent cell. In such embodiments, an inhibitor specific for BMP signaling can be a polypeptide or fragment thereof, an shRNA or siRNA directed against a BMP receptor, an antagonist antibody to a BMP receptor, or a small molecule antagonist of a BMP receptor. In one embodiment, the BMP receptor pathway is inhibited using an agent that inhibits BMP Type-I receptors. In one embodiment, the BMP Type I receptor inhibitor comprises LDN193189. Additional exemplary BMP Type I receptor inhibitors include, but are not limited to, LDN 212854, LDN 214117, Dorsomorphin dihydrochloride, DMH-1, DMH2, K 02288, and ML 347.

In some embodiments, the dosage range useful for a BMP pathway inhibitor (e.g., LDN193189) is between 0.1 and 5 μM, for example between 0.1 and 4 μM, between 0.1 and 3 μM, between 0.1 and 2 μM, between 0.1 and 1 μM, between 0.1 and 0.5 μM, between 0.1 and 2 μM, between 0.1 and 3 μM, between 0.1 and 4 μM, between 0.1 and 5 μM, between 0.5 and 2 μM, between 0.5 and 3 μM, between 0.75 and 1.5 μM, between 1 and 3 μM, between 1 and 5 μM, between 2 and 5 μM, between 3 and 5 μM, between 4 and 5 μM, or any range therebetween.

TGF-β signaling pathway modulation: In some embodiments, one or more TGF-β agonists are used to promote a particular differentiation step in a process of generating a desired cell type from a stem cell in vitro (e.g., from an iPS cell). In such embodiments, an activating agent specific for TGF-β signaling can be a TGF-β polypeptide or an active fragment thereof, a fusion protein comprising a TGF-β polypeptide or an active fragment thereof, an agonist antibody to a TGF-β receptor, or a small molecule agonist of a TGF-β receptor.

The Transforming growth factor beta (TGF-β) signaling pathway is involved in many cellular processes in both the adult organism and the developing embryo including cell growth, cell differentiation, apoptosis, cellular homeostasis and other cellular functions. TGF-β superfamily ligands bind to a type II receptor, which recruits and phosphorylates a type I receptor. The type I receptor then phosphorylates receptor-regulated SMADs (R-SMADs) which then bind the coSMAD SMAD4. R-SMAD/coSMAD complexes accumulate in the nucleus where they act as transcription factors and participate in the regulation of target gene expression.

TGF-β1 is a prototypic member of a family of cytokines including the TGF-βs, activins, inhibins, bone morphogenetic proteins and Mullerian-inhibiting substance. Smad proteins are exemplary downstream signal transduction factors in the TGF-beta pathway and therefore, in some embodiments, can be activated directly to effect differentiation to a human ameloblast (e.g., by treating a cell with an activator of a Smad protein). Exemplary Smad activators include, but are not limited to, Smad proteins or functional peptides or fragments thereof (e.g., Smad1, Smad5, Smad8), BMP2, BMP4, and Mullerian inhibiting substance (MIS). Activin ligands transduce signals in a manner similar to TGF-β ligands. Activins bind to and activate ALK receptors, which in turn phosphorylate Smad proteins such as Smad2 and Smad3. The consequent formation of a hetero-Smad complex with Smad4 results in the activin-induced regulation of gene transcription.

In some embodiments, the dosage range useful for TGF-β or an agonist that binds TGF-β receptors is between 200 nM and 1000 nM, for example, between 200 nM and 900 nM, between 200 nM and 800 nM, between 200 nM and 700 nM, between 200 nM and 600 nM, between 200 nM and 500 nM, between 200 nM and 400 nM, between 200 nM and 300 nM, between 300 nM and 1000 nM, between 400 nM and 1000 nM, between 500 nM and 1000 nM, between 600 nM and 1000 nM, between 700 mM and 1000 nM, between 800 nM and 1000 nM, between 900 nM and 1000 nM, between 500 nM and 900 nM, between 600-800 nM, between 700-900 nM, between 750 and 850 nM, between 600 and 900 nM or any range therebetween.

Wnt pathway modulation: Without wishing to be bound by theory, Wnt proteins and their cognate receptors signal through at least two distinct intracellular pathways. The “canonical” Wnt signaling pathway, (referred to herein as the Wnt/β-catenin pathway) involves Wnt signaling via β-catenin to activate transcription through TCF-related proteins (van de Wetering et al. (2002) Cell 109 Suppl: S13-9; Moon et al. (2002) Science 296(5573): 1644-6). A non-canonical alternative pathway exists, in which Wnt activates protein kinase C (PKC), calcium/calmodulin-dependent kinase II (CaMKII), JNK and Rho-GTPases (Veeman et al. (2003) Dev Cell 5(3): 367-77), and is often involved in the control of cell polarity.

Wnt agonists: Provided herein are methods for differentiating iPSCs to ameloblasts comprising contacting a cell with a Wnt agonist.

As used herein, the term “Wnt agonist” refers to any agent that activates the Wnt/β-catenin pathway, for example antagonists or inhibitors of GSK-3β activity, or inhibits the activity and/or expression of inhibitors of Wnt/β-catenin signaling. A Wnt activating agent as used herein can enhance signaling through the Wnt/β-catenin pathway at any point along the pathway, for example, but not limited to increasing the expression and/or activity of Wnt, or β-catenin or Wnt dependent genes and/or proteins, and decreasing the expression and/or activity of endogenous inhibitors of Wnt and/or β-catenin or decreasing the expression and/or activity of endogenous inhibitors of components of the Wnt/β-catenin pathway, for example decreasing the expression of GSK-3β.

Some non-limiting examples of Wnt pathway agonists include the GSK-3β antagonist CHIR99021 (6-[[2-[[4-(2,4-dichlorophenyl)-5-(5-methyl-1H-imidazol-2-yl)-2-pyrimidinyl]amino]ethyl]amino]-3-pyridinecarbonitrile), 2-amino-4-[3,4-(methylenedioxy)benzyl-amino]-6-(3-methoxyphenyl)pyrimidine, BIO ((2′Z,3′E)-6-Bromoindirubin-3′-oxime), 5-(Furan-2-yl)-N-(3-(1H-imidazol-1-yl)propyl)-1,2-oxazole-3-carboxamide, lithium carbonate, lithium chloride, CHIR98014 (e.g., 6-N-[2-[[4-(2,4-Dichlorophenyl)-5-imidazol-1-ylpyrimidin-2-yl]amino]ethyl]-3-nitropyridine-2,6-diamine dihydrochloride), and SKL2001.

In some embodiments, the dosage range useful for a Wnt agonist (e.g., CHIR99021) is between 0.1 and 10 μM, for example, between 0.1 and 9 μM, between 0.1 and 8 μM, between 0.1 and 7 μM, between 0.1 and 6 μM, between 0.1 and 5 μM, between 0.1 and 4 μM, between 0.1 and 3 μM, between 0.1 and 2 μM, between 0.1 and 1 μM, between 1.5 and 10 μM, between 2 and 6 μM, between 4 and 6 μM, between 4.5 and 6.5 μM, between 1 and 9 μM, between 2 and 9 μM, between 3 and 9 μM, between 4 and 9 μM, between 5 and 9 μM, between 6 and 9 μM, between 7 and 9 μM, between 8 and 9 μM, or any integer therebetween.

In some embodiments, the dose of a Wnt agonist (e.g., CHIR99021) is e.g., at least 0.1 μM, at least 0.5 μM, at least 1 μM, at least 1.1 μM, at least 1.2 μM, at least 1.3 μM, at least 1.4 μM, at least 1.5 μM, at least 1.6 μM, at least 1.7 μM, at least 1.8 μM, at least 1.9 μM, at least 2 μM, at least 2.5 μM, at least 3 μM, at least 4 μM, at least 5 μM, at least 6 μM, at least 7 μM, at least 8 μM, at least 9 μM, at least 10 μM, or more.

Epidermal Growth Factor: In some embodiments, epidermal growth factor (EGF) is used with the methods to generate ameloblasts as described herein. In some embodiments, the dosage range useful for epidermal growth factor is between 10 and 800 pM, for example between 10 and 700 pM, between 10 and 600 pM, between 10 and 500 pM, between 10 and 400 pM, between 10 and 300 pM, between 10 and 200 pM, between 10 and 100 pM, between 100 and 800 pM, between 200 and 800 pM, between 300 and 800 pM, between 400 and 800 pM, between 500 and 800 pM, between 600 and 800 pM, between 700 and 800 pM, between 400 and 600 pM, between 450 and 650 pM, between 500 and 600 pM, between 400 and 500 pM, or any dose therebetween.

Neurotrophin-4: In some embodiments, neurotrophin-4 is used with the methods to generate ameloblasts as described herein. In some embodiments, the dosage range useful for neurotrophin-4 is between 0.1 and 5 μM, for example, between 0.1 and 4 μM, between 0.1 and 3 μM, between 0.1 and 2 μM, between 0.1 and 1 μM, between 0.1 and 0.5 μM, between 0.5 and 3 μM, between 0.5 and 2 μM, between 0.5 and 1 μM, between 1 and 2 μM, between 1.5 and 2 μM, between 1 and 1.5 μM, between 2 and 5 μM, between 3 and 5 μM, between 4 and 5 μM, between 3 and 4 μM, or any dosage range therebetween.

### Scaffold and Matrix Material

Various embodiments of the compositions and methods described herein employ a scaffold seeded with progenitor cells, epithelial cells, mesenchymal cells, ameloblast precursors, ameloblasts, a combination thereof or an organoid comprising ameloblasts. In some embodiments, the ameloblasts or ameloblast organoids are grown in or on a scaffold in the presence of a mineralizing solution. Accordingly, mineralized materials can be produced in or on the scaffold. The shape and characteristics of the scaffold can be chosen so as to provide a desired framework for generating a 3D ameloblast organoid or in a shape that can be mineralized and implanted into a subject.

A scaffold can be fabricated with any matrix material recognized as useful by the skilled artisan. A matrix material can be a biocompatible material that generally forms a porous, microcellular scaffold, which provides a physical support for cells migrating thereto. Such matrix materials can: allow cell attachment and migration; deliver and retain cells and biochemical factors; enable diffusion of cell nutrients and expressed products; or exert certain mechanical and biological influences to modify the behavior of the cell phase. The matrix material generally forms a porous, microcellular scaffold of a biocompatible material that provides a physical support and an adhesive substrate for growth of cells during in vitro or in vivo culturing.

The matrix comprising the scaffold can have an adequate porosity and an adequate pore size so as to facilitate cell growth and diffusion throughout the whole structure of both cells and nutrients. In embodiments where the scaffold is implanted, the matrix can be biodegradable providing for absorption of the matrix by the surrounding tissues (if implanted), which can eliminate the necessity of a surgical removal. The rate at which degradation occurs can coincide as much as possible with the rate of tissue or organ formation. Thus, while cells are fabricating their own natural structure around themselves (e.g., dentin, cementum, enamel), the matrix is able to provide structural integrity and eventually break down, leaving the neotissue, newly formed tissue or organ which can assume the mechanical load. The matrix can be an injectable matrix in some configurations. The matrix can be delivered to a tissue using minimally invasive endoscopic procedures.

The scaffold can comprise a matrix material having different phases of viscosity. For example, a matrix can have a substantially liquid phase or a substantially gelled phase. The transition between phases can be stimulated by a variety of factors including, but not limited to, light, chemical, magnetic, electrical, and mechanical stimulus. For example, the matrix can be a thermosensitive matrix with a substantially liquid phase at about room temperature and a substantially gelled phase at about body temperature. The liquid phase of the matrix can have a lower viscosity that provides for optimal distribution of growth factors or other additives and injectability, while the solid phase of the matrix can have an elevated viscosity that provides for matrix retention at or within the target tissue.

The scaffold can comprise a matrix material formed of synthetic polymers. Such synthetic polymers include, but are not limited to, polyurethanes, polyorthoesters, polyvinyl alcohol, polyamides, polycarbonates, polyvinyl pyrrolidone, marine adhesive proteins, cyanoacrylates, analogs, mixtures, combinations and derivatives of the above. Alternatively, the matrix can be formed of naturally occurring biopolymers. Such naturally occurring biopolymers include, but are not limited to, fibrin, fibrinogen, fibronectin, collagen, and other suitable biopolymers. Also, the matrix can be formed from a mixture of naturally occurring biopolymers and synthetic polymers.

The scaffold can include one or more matrix materials including, but not limited to, a collagen gel, a polyvinyl alcohol sponge, a poly(D,L-lactide-co-glycolide) fiber matrix, a polyglactin fiber, a calcium alginate gel, a polyglycolic acid mesh, polyester (e.g., poly-(L-lactic acid) or a polyanhydride), a polysaccharide (e.g. alginate), polyphosphazene, polyacrylate, or a polyethylene oxide-polypropylene glycol block copolymer. Matrices can be produced from proteins (e.g. extracellular matrix proteins such as fibrin, collagen, and fibronectin), polymers (e.g., polyvinylpyrrolidone), or hyaluronic acid. Synthetic polymers can also be used, including bioerodible polymers (e.g., poly(lactide), poly(glycolic acid), poly(lactide-co-glycolide), poly(caprolactone), polycarbonates, polyamides, polyanhydrides, polyamino acids, polyortho esters, polyacetals, polycyanoacrylates), degradable polyurethanes, non-erodible polymers (e.g., polyacrylates, ethylene-vinyl acetate polymers and other acyl substituted cellulose acetates and derivatives thereof), non-erodible polyurethanes, polystyrenes, polyvinyl chloride, polyvinyl fluoride, poly(vinylimidazole), chlorosulphonated polyolifins, polyethylene oxide, polyvinyl alcohol, Teflon®, or nylon.

The scaffold can further comprise any other bioactive molecule, for example an antibiotic or an additional chemotactic growth factor or another osteogenic, dentinogenic, amelogenic, or cementogenic growth factor. In some embodiments, the scaffold is strengthened, through the addition of, e.g., human serum albumin (HSA), hydroxyethyl starch, dextran, or combinations thereof. Suitable concentrations of these compounds for use in the compositions of the application are known to those of skill in the art, or can be readily ascertained without undue experimentation. The concentration of compound in the scaffold will vary with the nature of the compound, its physiological role, and desired therapeutic or diagnostic effect. A therapeutically effective amount is generally a sufficient concentration of therapeutic agent to display the desired effect without undue toxicity. The compound can be incorporated into the scaffold or matrix material by any known method. In some embodiments, the compound is imbedded in a gel, e.g., a collagen gel incorporated into the pores of the scaffold or matrix material.

Alternatively, chemical modification methods can be used to covalently link the compound to a matrix material. The surface functional groups of the matrix can be coupled with reactive functional groups of the compound to form covalent bonds using coupling agents well known in the art such as aldehyde compounds, carbodiimides, and the like. Additionally, a spacer molecule can be used to gap the surface reactive groups and the reactive groups of the biomolecules to allow more flexibility of such molecules on the surface of the matrix. Other similar methods of attaching biomolecules to the interior or exterior of a matrix will be known to one of skill in the art.

Pores and channels of the scaffold can be engineered to be of various diameters. For example, the pores of the scaffold can have a diameter range from micrometers to millimeters. In some embodiments, the pores of the matrix material include microchannels. Microchannels generally have an average diameter of about 0.1 μm to about 1,000 μm, e.g., about 50 μm to about 500 μm (for example about 100 μm, 150 μm, about 200 μm, about 250 μm, about 300 μm, about 350 μm, about 400 μm, about 450 μm, about 500 μm, or about 550 μm). One skilled in the art will understand that the distribution of microchannel diameters can have any distribution including a normal distribution or a non-normal distribution. In some embodiments, microchannels are a naturally occurring feature of the matrix material(s). In other embodiments, microchannels are engineered to occur in the matrix materials.

Several methods can be used for fabrication of porous scaffolds, including particulate leaching, gas foaming, electrospinning, freeze drying, foaming of ceramic from slurry, and the formation of polymeric sponge. Other methods that can be used for fabrication of porous scaffolds include computer aided design (CAD) and synthesizing the scaffold with a bioplotter (e.g., solid freeform fabrication) (e.g., Bioplotter™, EnvisionTec, Germany).

Biologic drugs that can be added to the scaffold or matrix compositions include immunomodulators and other biological response modifiers. A biological response modifier generally encompasses a biomolecule (e.g., peptide, peptide fragment, polysaccharide, lipid, antibody) that is involved in modifying a biological response, such as the immune response or tissue or organ growth and repair, in a manner that enhances a particular desired therapeutic effect, for example, the cytolysis of bacterial cells or the growth of tissue- or organ-specific cells or vascularization. Biologic drugs can also be incorporated directly into the matrix component. Those of skill in the art will know, or can readily ascertain, other substances which can act as suitable non-biologic and biologic drugs.

Compositions described herein can also be modified to incorporate a diagnostic agent, such as a radiopaque agent. Such compounds include barium sulfate as well as various organic compounds containing iodine. Examples of these latter compounds include iocetamic acid, iodipamide, iodoxamate meglumine, iopanoic acid, as well as diatrizoate derivatives, such as diatrizoate sodium. Other contrast agents that can be utilized in the compositions of the invention can be readily ascertained by those of skill in the art and can include, for example, the use of radiolabeled fatty acids or analogs thereof.

The concentration of an agent in the composition will vary with the nature of the compound, its physiological role, and desired therapeutic or diagnostic effect. A therapeutically effective amount is generally a sufficient concentration of therapeutic agent to display the desired effect without undue toxicity. A diagnostically effective amount is generally a concentration of diagnostic agent which is effective in allowing the monitoring of the integration of a tissue graft, while minimizing potential toxicity. In any event, the desired concentration in a particular instance for a particular compound is readily ascertainable by one of skill in the art.

### Dental Repair Compositions

Provided herein, in some embodiments, are dental repair compositions for use in the treatment of enamel disorders. In one embodiment, such dental repair compositions comprise ameloblastin produced by in vitro differentiated ameloblast cells. In other embodiments, the dental repair composition comprises enamel produced by in vitro differentiate ameloblasts.

In some embodiments, the dental repair composition further comprises calcium phosphate or hydroxyapatite. Other components of such dental repair compositions can include one or more of amelogenin (AMELX), or enamelin (ENAM).

Dental repair compositions can be in the form of an implant (e.g., a tooth implant) or can be a dental composite for application to an existing tooth with cracked, chipped, demineralized or otherwise dysfunctional enamel.

### Treatment of Enamel Disorders

The methods and compositions provided herein relate to the generation and use of human ameloblasts. Accordingly, provided herein are methods for the treatment and prevention of a disease or disorder associated with an enamel disorder in a subject in need thereof. The methods described herein can be used to treat, ameliorate, prevent or slow the progression of a number of enamel-related diseases or their symptoms, such as those resulting in irreversible damage or demineralization to dental enamel.

The methods described herein can also be used to treat or ameliorate acute or chronic enamel issues or their symptoms or complications, including enamel hypoplasia, amelogenesis imperfecta, celiac disease-associated enamel problems, enamel erosion, enamel demineralization, dental caries, enamel fractures, bruxism, enamel abrasion, chronic bilirubin encephalopathy, erythropoietic porphyria, or tetracycline-induced enamel staining or loss.

As used herein, the terms “administering,” “introducing” and “transplanting” are used interchangeably in the context of the placement of cells, e.g. ameloblasts or organoids comprising ameloblasts, as described herein into a subject, by a method or route which results in at least partial localization of the introduced cells at a desired site, such as a site of injury or repair, such that a desired effect(s) is produced. The cells e.g. ameloblasts, or their differentiated progeny can be implanted directly to the gums or the mouth, or alternatively be administered by any appropriate route which results in delivery to a desired location in the subject where at least a portion of the implanted cells or components of the cells remain viable. The period of viability of the cells after administration to a subject can be as short as a few hours, e.g., twenty-four hours, to a few days, to as long as several years, i.e., long-term engraftment.

When provided prophylactically, ameloblast cells or organoids as described herein can be administered to a subject in advance of any symptom of enamel loss or impaired enamel development. Accordingly, the prophylactic administration of ameloblasts or organoids thereof serves to prevent enamel loss or impaired enamel production.

When provided therapeutically, ameloblasts are provided at (or after) the onset of a symptom or indication of an enamel disorder, e.g., upon the detection of one or more sites of enamel loss.

In some embodiments of the aspects described herein, the ameloblasts or organoids thereof being administered according to the methods described herein comprise allogeneic ameloblasts obtained from one or more donors. As used herein, “allogeneic” refers to an ameloblast derived from somatic cell derived-iPSCs obtained from one or more different donors of the same species, where the genes at one or more loci are not identical. For example, an ameloblast or organoid thereof being administered to a subject can be derived from umbilical cord blood obtained from one more unrelated donor subjects, or from one or more non-identical siblings. In some embodiments, syngeneic ameloblasts can be used, such as those obtained from genetically identical animals, or from identical twins. In other embodiments of this aspect, the ameloblasts are autologous cells; that is, the ameloblasts are differentiated from stem cells, e.g., iPS cells, derived from a subject and administered to the same subject, i.e., the donor and recipient are the same.

### Mini-Binders

As used herein, mini-protein binders (mb) are computer designed proteins which bind to specific sequences of proteins in order to alter their function. In some embodiments, mini-protein binders bind to specific sequences of proteins in order to inhibit the function of a native protein.

In some embodiments, the mini-protein binder is directed toward FGFR (FGFR2 mini-binder, FGFR_mb) and/or EGFR (EGFR mini-binder, EGFR mb). In some embodiments, the FGFR mini-protein binder contains the binding sequence MGDRRKEMDKVYRTAYKRITSTPDKEKRKEVVKEATEQLRRIAKDEEEKKKAAYMISFLKTLG LEHHHHHH (SEQ ID NO. 1). The mini-protein binder can contain a detection tag on either the N- or C-terminus of the binding sequence. In some embodiments, the detection tag is MSHHHHHHHHSENLYFQSGGG (SEQ ID NO. 2).

A variant amino acid relating to a mini-protein binder can be at least 85%, at least 87%, at least 90%, at least 91%, at least 92%, at least 93%, at least 94%, at least 95%, at least 96%, at least 97%, at least 98%, at least 99%, or more, identical to a native or reference sequence of the mini-protein binder. The degree of homology (percent identity) between a reference and a mutant sequence can be determined, for example, by comparing the two sequences using freely available computer programs commonly employed for this purpose on the world wide web (e.g., BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

In some embodiments, the expression of a biomarker(s), target(s), or gene/polypeptide described herein is/are tissue-specific. In some embodiments, the expression of a biomarker(s), target(s), or gene/polypeptide described herein is/are global. In some embodiments, the expression of a biomarker(s), target(s), or gene/polypeptide described herein is systemic.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

Techniques for designing and using mini-protein binders are established and include, for example, those disclosed by Cao et al. (2022, ‘Design of protein-binding proteins from the target structure alone’, Nature, 605: 551-60), and International Application No. PCT/US22/73590, which are herein incorporated by reference in their entireties.

### Pharmaceutically Acceptable Carriers

In one aspect, the methods of introducing or replacing enamel-producing cells in a subject as described herein involve the use of therapeutic compositions comprising ameloblasts or organoids comprising ameloblasts. Therapeutic compositions contain a physiologically tolerable carrier together with the cell composition and optionally at least one additional bioactive agent as described herein, dissolved or dispersed therein as an active ingredient. In a preferred embodiment, the therapeutic composition is not substantially immunogenic when administered to a mammal or human patient for therapeutic purposes, unless so desired. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset, transplant rejection, allergic reaction, and the like. A pharmaceutically acceptable carrier will not promote the raising of an immune response to an agent with which it is admixed, unless so desired. The preparation of a composition that contains active ingredients dissolved or dispersed therein is well understood in the art and need not be limited based on formulation. Typically, such compositions are prepared as injectable either as liquid solutions or suspensions, however, solid forms suitable for solution, or suspensions, in liquid prior to use can also be prepared.

In general, the human ameloblasts or organoids thereof described herein are administered as a suspension with a pharmaceutically acceptable carrier. One of skill in the art will recognize that a pharmaceutically acceptable carrier to be used in a cell composition will not include buffers, compounds, cryopreservation agents, preservatives, or other agents in amounts that substantially interfere with the viability of the cells to be delivered to the subject. A formulation comprising cells can include e.g., osmotic buffers that permit cell membrane integrity to be maintained, and optionally, nutrients to maintain cell viability or enhance engraftment upon administration. Such formulations and suspensions are known to those of skill in the art and/or can be adapted for use with the human ameloblasts as described herein using routine experimentation.

A cell composition can also be emulsified or presented as a liposome composition, provided that the emulsification procedure does not adversely affect cell viability. The cells and any other active ingredient can be mixed with excipients which are pharmaceutically acceptable and compatible with the active ingredient and in amounts suitable for use in the therapeutic methods described herein.

Additional agents included in a cell composition as described herein can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active compound used in the cell compositions as described herein that is effective in the treatment of a particular disorder or condition will depend on the nature of the disorder or condition, and can be determined by standard clinical techniques.

### Administration and Efficacy

Provided herein are methods for treating a disease or disorder comprising enamel problems (e.g., enamel demineralization or impaired enamel production during development) by administering human ameloblasts, organoids comprising ameloblasts, ameloblastin or a dental repair composition comprising ameloblastin or enamel prepared as described herein to a subject in need thereof.

In some embodiments, the methods of treating a disease or disorder comprise first diagnosing a subject with a dental enamel problem that requires treatment. In other embodiments, the degree of dental enamel loss or impaired enamel production during development is first assessed using one or more measured or measurable parameters including clinically detectable markers of disease, for example, enamel discoloration, tooth sensitivity to heat/cold, chips or cracks in enamel, scanning electron microscopy, stereo microscopy, white light 3D profilometry, and atomic force microscopy. It will be understood, however, that the dosage of administered ameloblasts, organoids thereof, or enamel produced by ameloblasts and total usage of the compositions and formulations as disclosed herein will be decided by the attending clinician within the scope of sound medical judgment. The exact amount required will vary depending on factors such as the type of disease being treated.

The term “effective amount” as used herein refers to the amount of a population of ameloblasts or organoids thereof needed to alleviate at least one or more symptom of impaired dental enamel, and relates to a sufficient amount of a composition to provide the desired effect, e.g., treat a subject to augment or replace enamel. The term “therapeutically effective amount” therefore refers to an amount of, e.g., human ameloblasts or an organoid comprising ameloblasts that is sufficient to promote a particular effect when administered to a typical subject, such as one who has or is at risk for enamel loss or impaired enamel production. An effective amount as used herein would also include an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate “effective amount” can be determined by one of ordinary skill in the art using routine experimentation.

In some embodiments, the subject is first diagnosed as having a disease or disorder affecting dental enamel prior to administering the cells or other compositions as described herein according to the methods described herein. In some embodiments, the subject is first diagnosed as being at risk of developing a disease or disorder comprising enamel loss or impaired enamel production prior to administering the cells or other compositions as described herein.

For use in the various aspects described herein, an effective amount of human ameloblast cells can comprise at least 10² ameloblast cells, at least 5×10² ameloblast cells, at least 10³ ameloblast cells, at least 5×10³ ameloblast cells, at least 10⁴ ameloblast cells, at least 5×10⁴ ameloblast cells, at least 10⁵ ameloblast cells, at least 2×10⁵ ameloblast cells, at least 3×10⁵ ameloblast cells, at least 4×10⁵ ameloblast cells, at least 5×10⁵ ameloblast cells, at least 6×10⁵ ameloblast cells, at least 7×10⁵ ameloblast cells, at least 8×10⁵ ameloblast cells, at least 9×10⁵ ameloblast cells, at least 1×10⁶ ameloblast cells, at least 2×10⁶ ameloblast cells, at least 3×10⁶ ameloblast cells, at least 4×10⁶ ameloblast cells, at least 5×10⁶ ameloblast cells, at least 6×10⁶ ameloblast cells, at least 7×10⁶ ameloblast cells, at least 8×10⁶ ameloblast cells, at least 9×10⁶ ameloblast cells, at least 10⁷ ameloblast cells, at least 2×10⁷ ameloblast cells, at least 3×10⁷ ameloblast cells, at least 4×10⁷ ameloblast cells, at least 5×10⁷ ameloblast cells, at least 6×10⁷ ameloblast cells, at least 7×10⁷ ameloblast cells, at least 8×10⁷ ameloblast cells, at least 9×10⁷ ameloblast cells, at least 1×10⁸ ameloblast cells, at least 2×10⁸ ameloblast cells, at least 3×10⁸ ameloblast cells, at least 4×10⁸ ameloblast cells, at least 5×10⁸ ameloblast cells, at least 6×10⁸ ameloblast cells, at least 7×10⁸ ameloblast cells, at least 8×10⁸ ameloblast cells, at least 9×10⁸ ameloblast cells, at least 1×10⁹ ameloblast cells, at least 2×10⁹ ameloblast cells, at least 3×10⁹ ameloblast cells, at least 4×10⁹ ameloblast cells, at least 5×10⁹ ameloblast cells, at least 6×10⁹ ameloblast cells, at least 7×10⁹ ameloblast cells, at least 8×10⁹ ameloblast cells, at least 9×10⁹ ameloblast cells, or more. The ameloblast cells can be derived from one or more donors, or can be obtained from an autologous source. In some embodiments of the aspects described herein, the ameloblast cells are expanded in culture prior to administration to a subject in need thereof.

Exemplary modes of administration for use in the methods described herein include, but are not limited to, local administration (e.g., using a paste), injection and implantation (with or without a scaffold material).

In some embodiments of the aspects described herein, one or more routes of administration are used in a subject to achieve distinct effects. For example, ameloblasts can be administered to a subject by both implantation and local administration routes for treating or repairing tooth enamel. In such embodiments, different effective amounts of the ameloblast cells can be used for each administration route.

In some embodiments, additional agents to aid in treatment of the subject can be administered before or following treatment with the ameloblast cells described herein. Such additional agents can be used to prepare the gum tissue for administration of the ameloblasts cells. Alternatively, the additional agents can be administered after the ameloblast cells to support the engraftment and growth of the administered cell in the gum tissue.

The efficacy of treatment can be determined by the skilled clinician (e.g., a dental assistant or dentist). However, a treatment is considered “effective treatment,” as the term is used herein, if any one or all of the symptoms, or other clinically accepted symptoms or markers of thin enamel, weakened enamel or demineralized enamel are reduced, e.g., by at least 10% following treatment with a composition comprising or produced by human ameloblast cells as described herein. Methods of measuring these indicators are known to those of skill in the art and/or described herein.

Indicators of an enamel disease, disorder, or injury include one or more of, e.g., pain, sensitivity to temperature, cracks, chips, caries, discoloration, or by clinical means such as scanning electron microscopy, stereo microscopy, white light 3D profilometry, and atomic force microscopy.

### Screening Platforms Using Stem Cell-Derived Ameloblasts

The matured ameloblasts or organoids thereof prepared as described herein provide a platform for the study or evaluation of the likely effects of known or experimental drugs that can impact enamel production. In addition, the matured ameloblasts or organoids thereof prepared as described herein can be used to assess functional changes in response to genomic modifications or mutations. In particular, ameloblasts or organoids produced as described herein derived from a subject having a given disease can be used to model a given disease in a dish and to screen for agents or genomic modification that regulate disease. By screening with, for example, a library or collection of potential drugs or agents, ameloblasts or organoids thereof prepared and matured as described herein can also be used to identify new drugs with beneficial effects on ameloblast viability or enamel production.

Ameloblasts or organoids thereof derived from normal donor cells can provide useful information in both situations, and ameloblasts derived from donors with dental enamel demineralization or other enamel issues, or derived from cells engineered to mimic a dental disease or disorder can be very useful in identifying new drugs or agents to treat such diseases.

Screening assays can also be used in combination with mutagenesis assays to test for correction of disease in patient cell lines, ameloblasts derived from a patient having enamel dysfunction, or in ameloblast organoids prepared as described herein. Such mutations can be introduced using any known genome modification system including, but not limited to, base or prime genome editors, CRISPR/Cas, zinc finger nucleases (ZFN), transcription activator-like effector nucleases (TALENs), and the like and their effect on function can be assessed.

In either instance, the evaluation of functional or structural parameters as described herein or as known in the art can be informative with regard to the effects of a given agent or given mutation. In general, such assays comprise contacting ameloblasts prepared and matured as described herein with an agent and measuring one or more parameters of the ameloblasts described herein as an indicator of the agent's effect(s) or introducing a mutation to the genome of the ameloblast and measuring one or more functional parameters. Where effects are observed, dose responses can also be evaluated by varying the concentration of the agent and/or the duration of contacting.

Accordingly, stem cell-derived ameloblasts prepared and matured as described herein can be used to identify an agent, evaluate an agent, or evaluate a genomic modification for its effect on parameters such as expression of markers, cell viability, mineral deposition, enamel production or other parameters described herein or known in the art.

In some embodiments, ameloblasts or organoids comprising ameloblasts prepared as described herein can be used to identify targets for genetic manipulation including, but not limited to gene editing to modify a disease phenotype. As a non-limiting example, ameloblast-comprising organoids prepared as described herein using iPS cells derived from a subject with amelogenesis imperfecta can be subjected to a random mutagenesis or base-editing regimen and assayed for changes in expression of ameloblastin or other markers of enamel production. Clones exhibiting favorable changes in phenotype can be analyzed to determine the genetic change(s) involved in bringing about the phenotypic change, thereby identifying gene targets for therapeutic manipulation, including but not limited to targeted genomic modification.

In some embodiments, stem cell-derived ameloblasts can be used in assays to screen agents, selected from small molecules, nucleic acids or analogues thereof, aptamers; proteins or polypeptides or analogues or fragments thereof, among other agents for effects, detrimental or beneficial, on the cells.

In some embodiments, the agent is an agent of interest including known and unknown compounds that encompass numerous chemical classes, primarily organic molecules, which may include organometallic molecules, inorganic molecules, genetic sequences, etc. Candidate agents also include organic molecules comprising functional groups necessary for structural interactions, particularly hydrogen bonding, and typically include amine, carbonyl, hydroxyl or carboxyl groups, frequently more than one of such functional chemical groups. The candidate agents often comprise cyclic carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Also included as agents are pharmacologically active drugs, genetically active molecules, etc. Compounds of interest include, for example, growth factors or recombinant growth factors and fragments and variants thereof. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Water, Salts and Ions; Drugs Affecting Renal Function and Electrolyte Metabolism; Drugs Affecting Gastrointestinal Function; Chemotherapy of Microbial Diseases; Chemotherapy of Neoplastic Diseases; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

Compounds, including candidate agents, can be obtained from a variety of sources including libraries of synthetic or natural compounds. Various means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Candidate agents include all of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g., ground water, sea water, etc.; biological samples, e.g. lysates prepared from crops, tissue samples, etc.; manufacturing samples, e.g. time course during preparation of pharmaceuticals; as well as libraries of compounds prepared for analysis; and the like.

In some embodiments, the effect of the agent or the effect of a given genomic modification is determined by quantifiable parameters of stem cell-derived ameloblasts, such as expression of ameloblastin or amelogenin, mineral deposition, enamel production etc. In some embodiments, quantifiable parameters include differentiation, survival and regeneration of the stem cell-derived ameloblasts.

A plurality of assays comprising stem cell-derived ameloblasts can be run in parallel with different agent concentrations to obtain a differential response to the various concentrations. As known in the art, determining the effective concentration of an agent typically uses a range of concentrations resulting from 1:10, or other log scale, dilutions. The concentrations can be further refined with a second series of dilutions, if necessary. Typically, one of these concentrations serves as a negative control, i.e. at zero concentration or below the level of detection of the agent or at or below the concentration of agent that does not give a detectable change in the phenotype.

Optionally, the stem cell-derived ameloblasts (or organoids comprising such ameloblasts) used in the screen can be manipulated to express desired gene products.

### Kits

In some embodiments, the compositions as described herein can be prepared as a kit. In another embodiment, the kit further comprises an iPS cell or ES cell preparation, which can be metabolically active or frozen, and can optionally include reagents as described herein for differentiating cells from the iPS cell or ES cell preparation to an ameloblast phenotype.

In another embodiment, the kit comprises stem cell-derived ameloblasts, which can be metabolically active or frozen. In another embodiment, the kit and/or any of its constituents can be shipped and/or stored at ambient or room temperature, or at, e.g., 4° C.

In some embodiments, the iPS cells, ES cells, or stem cell-derived ameloblasts are human cells, rodent cells, canine cells, and the like.

In some embodiments, the present technology may be defined in any of the following numbered paragraphs:
1. A method of preparing an ameloblast culture, the method comprising, in order,
   a) contacting, in culture, an induced pluripotent stem cell (iPSC) with an activator of the Hedgehog pathway;
   b) adding Bone Morphogenetic Protein 4 (BMP4) to the culture of (a);
   c) adding an inhibitor of BMP type I receptors, a Wnt activator, Epidermal Growth Factor (EGF) and Neurotrophin-4 (NT4) to the culture of (b) and incubating to form oral epithelium cells; and
   d) adding BMP4 and transforming growth factor β1 (TGF-β1) to the culture of (c) and incubating to form ameloblasts.
2. The method of item 1, wherein the ameloblasts express ameloblastin.
3. The method of item 1 or item 2, wherein the cells are human.
4. The method of any one of items 1-3, wherein the iPSCs are seeded on tissue culture plates coated with an extracellular matrix composition.
5. The method of item 4, wherein the extracellular matrix composition comprises a natural or a synthetic extracellular matrix composition.
6. The method of any one of items 1-5, wherein the iPSCs are grown to confluence prior to step (a).
7. The method of item 6, wherein the iPSCs are cultured to confluence in mTeSR1 stem cell medium.
8. The method of any one of items 1-7, wherein the Hedgehog activator is smoothened agonist (SAG).
9. The method of any one of items 6-8, wherein confluent iPS cells are switched to medium comprising the Hedgehog activator at day zero of differentiation.
10. The method of item 8 or item 9, wherein SAG is added at 200 nM to 1 μM.
11. The method of any one of items 8-10, wherein SAG is added at 400 nM.
12. The method of any one of items 1-11, wherein addition of BMP4 step (b) is performed at day 3 of differentiation.
13. The method of any one of items 1-12, wherein addition of BMP4 step (b) adds BMP4 at 100 pM to 750 pM.
14. The method of any one of items 1-13, wherein addition of BMP4 step (b) adds BMP4 at 150 pM.
15. The method of any one of items 1-14, wherein cells are incubated with BMP4 of step (b) from day 3 until day 7 of differentiation.
16. The method of any one of items 1-15, wherein step (c) addition of an inhibitor of BMP type I receptors, a Wnt activator, EGF and NT4 is performed at day 8 of differentiation.
17. The method of any one of items 1-16, wherein the inhibitor of BMP type I receptors is LDN-193189.
18. The method of item 17, wherein the LDN-193189 is added at 100 nM to 5 μM.
19. The method of item 17 or 18, wherein LDN-193189 is added at 1 μM.
20. The method of any one of items 1-19, wherein the Wnt activator is a GSK-3 inhibitor.
21. The method of item 20, wherein the GSK-3 inhibitor is CHIR99021. 22. The method of item 21, wherein CHIR99021 is added at 0.5 μM to 50 μM.
23. The method of item 21 or 22, wherein the CHIR99021 is added at 5 μM.
24. The method of any one of items 1-23, wherein EGF is added at 50 pM to 5 nM.
25. The method of any one of items 1-24, wherein EGF is added at 500 pM.
26. The method of any one of items 1-25, wherein NT4 is added at 350 nM to 35 μM.
27. The method of any one of items 1-26, wherein NT4 is added at 3.5 μM.
28. The method of any one of items 1-27, wherein step (d) addition of BMP4 and TGF-β1 is performed at day 10 of differentiation.
29. The method of any one of items 1-27, wherein step (d) addition of BMP4 and TGF-β1 is performed when expression of one or more of PITX2, TBX1 and TP63 is detected in the differentiating culture.
30. The method of any one of items 1-29, wherein the BMP-4 added at step (d) is added at 30 pM to 3 nM.
31. The method of any one of items 1-30, wherein the BMP-4 added at step (d) is added at 300 pM.
32. The method of any one of items 1-31, wherein the TGF-β1 is added at 80 nM to 8 μM.
33. The method of any one of items 1-32, wherein the TGF-β1 is added at 800 nM.
34. The method of any one of items 1-33, wherein the incubating of step (d) is to day 16 or more of differentiation.
35. A cultured organoid comprising in an in vitro-differentiated ameloblast.
36. The cultured organoid of item 35, wherein the ameloblast is differentiated from an iPS cell.
37. The cultured ameloblast of item 35 or 36, wherein the ameloblast is human.
38. The cultured organoid of any one of items 35-37, wherein ameloblastin is secreted into a lumen in the organoid.
39. A tooth comprising a dental repair composition comprising ameloblastin produced by an in vitro-differentiated cell.
40. A tooth comprising a dental repair composition comprising enamel produced by an in vitro-differentiated cell.
41. The tooth of item 39 or 40, wherein the dental repair composition further comprises calcium phosphate or hydroxyapatite.
42. The tooth of any one of items 39-41, wherein the dental repair composition further comprises one or more of amelogenin and enamelin.
43. A dental repair composition comprising ameloblastin produced by an in vitro-differentiated cell.
44. A dental repair composition comprising enamel produced by an in vitro-differentiated cell.
45. The dental repair composition of item 43 or 44, which further comprises hydroxyapatite or calcium phosphate.
46. The dental repair composition of any one of items 43-45, which further comprises one or more of amelogenin and enamelin.
47. The composition of any one of items 43 to 46, wherein the in vitro-differentiated cell is an ameloblast differentiated from an iPS cell.
48. The composition of item 47, wherein the iPS cell is a human iPS cell.
49. A method of repairing a tooth, the method comprising contacting a tooth with a dental repair composition of any one of items 43-48.
50. A method of treating amelogenesis imperfecta, the method comprising administering a composition comprising an in vitro-differentiated ameloblast to a subject in need thereof.
51. The method of item 50, wherein the ameloblast is differentiated from an iPS cell.
52. The method of item 50 or 51, wherein the ameloblast is a human ameloblast.
53. The method of any one of items 50-52, wherein the iPS cell is derived from the subject.
54. A method of preparing a tooth enamel repair composition, the method comprising culturing an in vitro-differentiated ameloblast.
55. The method of item 54, wherein the ameloblast is differentiated from an iPS cell.
56. The method of item 54 or 55, wherein the ameloblast is human.
57. The method of any one of items 54-56, wherein the ameloblast is in an organoid.
58. A method of screening for an agent that modulates enamel production, the method comprising contacting an in vitro-differentiated ameloblast or an organoid comprising an in vitro differentiated ameloblast with a candidate agent, and detecting a change in ameloblastin expression.
59. The method of item 58, wherein the ameloblast is differentiated from an iPS cell.
60. The method of item 58 or 59, wherein the ameloblast is differentiated from an iPS cell by the method of any one of items 1-34.
61. The method of any one of items 58-60, wherein the iPS cell is derived from an individual with a defect in enamel production.
62. The method of item 61, wherein the defect in enamel production comprises amelogenesis imperfecta.

The technology described herein is further illustrated by the Examples which in no way should be construed as being further limiting.

### EXAMPLES

The following provides non-limiting Examples demonstrating and supporting the technology as described herein.

### Example 1

Tooth enamel is the hardest tissue in the human body. In addition to providing masticatory function, it protects the underlying dentin and dental pulp from mechanical, chemical, and microbiological damages that can lead to tooth loss. Unlike many other tissues, the adult human tooth does not regenerate enamel due to the absence of the enamel-secreting cell type, ameloblasts (Park et al., 2013), making enamel vulnerable to permanent damage or tooth loss. In addition to injury and damage, congenital genetic diseases such as Amelogenesis Imperfecta can also contribute to enamel loss. Ameloblasts are dental epithelial cells that secrete enamel protein matrix and deposit minerals to achieve hard and mature tooth enamel during human development (Jernvall and Thesleff, 2012). During tooth eruption in humans, ameloblasts undergo apoptosis (Park et al., 2013; Yajima-Himuro et al., 2014). Though almost all humans acquire some damage to the protective enamel shield as adulthood progresses, currently, humans do not have a way to regenerate ameloblasts (Fugolin and Pfeifer, 2017).

Although tooth development has been studied over several years (Yu and Klein, 2020), most of these excellent developmental and molecular studies have been conducted using murine models (Balic and Thesleff, 2015; Chiba et al., 2020; Krivanek et al., 2020; Sharir et al., 2019; Thesleff, 2014) which presents several challenges when applied to human development (Balic, 2019; Fresia et al., 2021; Hovorakova et al., 2018). For example, mouse incisors undergo continuous regeneration due to a population of epithelial stem cells in the labial cervical loop that allows for continued enamel formation throughout life (Harada et al., 2002). Since this regenerative process does not occur in adult human teeth, it is critical to understand tooth differentiation during early human developmental stages. In addition, the enamel organ, which ultimately gives rise to ameloblasts, is comprised of multiple populations of support cells, including the stellate reticulum and the inner and outer enamel epithelium (Nanci and TenCate, 2018). These support cells are thought to be essential for ameloblast function (Harada et al., 2006; Maas and Bei, 1997; Nakamura et al., 1991); however, it is not understood how they are mechanistically involved in ameloblast differentiation and functional maturation. Animal studies have suggested several pathways in driving and regulating this communication, such as the hedgehog (HH)(Koyama et al., 2001), NOTCH (Harada et al., 2006), and FGF (Takamori et al., 2008) pathways. However, the temporal regulation and the extent to which these pathways originate from support cells are not clearly understood since these cells are poorly studied in humans. Dissecting human tooth development at the single-cell level can capture the patterns of gene expression that characterize small populations of support cells that are involved in the differentiation.

In order to understand human tooth development and to facilitate the regeneration of human tooth structures in the future, single-cell combinatorial indexing RNA sequencing (sci-RNA-seq)(Cao et al., 2019) technology has been utilized to study human fetal tooth development at 9-22 gestational weeks (gw). Through computational analysis of the sci-RNA-seq data, it has established for the first time a spatiotemporal single-cell atlas for developing human teeth that includes both the epithelial and mesenchymal cell types. The computational studies established human-specific transcriptional profiles for subtypes of the developing tooth and revealed novel branches in the developmental trajectories of both mesenchymal and epithelial-derived tissues, as well as previously undescribed populations of epithelial support tissues. From the studies, it was able to be identified in developing human tissue, the subodontoblast, a proposed novel odontoblast progenitor. Further, the critical signaling pathways were able to be defined and induced that drove changes in cell fate along the developmental trajectory of ameloblasts. This expedited the development of a 3D organoid that exhibits ameloblasts polarized towards odontoblast-like cells. This 3D organoid shows mineralization (calcium deposition) and expression of Ameloblastin, Amelogenin and Enamelin. Hence, the term Enamel Organoid has been coined to describe this new class of organoids.

These studies enhance our understanding of the regulatory mechanism controlling the differentiation process of dental tissues and lay the groundwork toward the development of disease models and regenerative approaches.

### Results

### A Single-Cell Atlas of the Developing Human Fetal Odontogenic Tissues

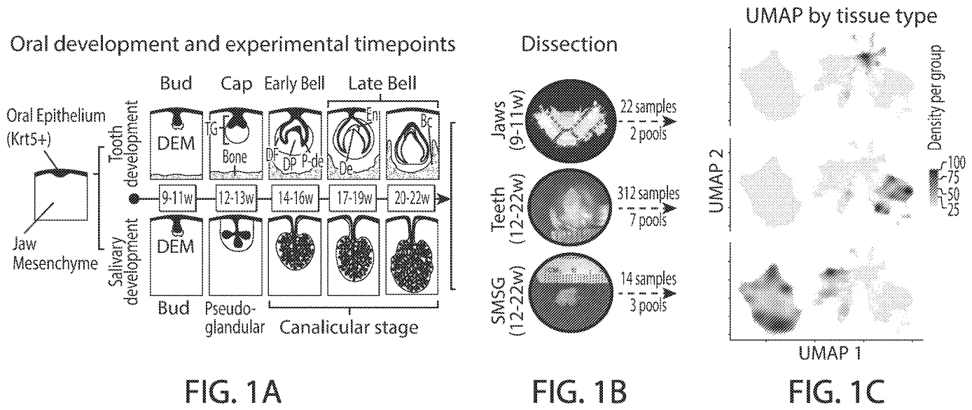

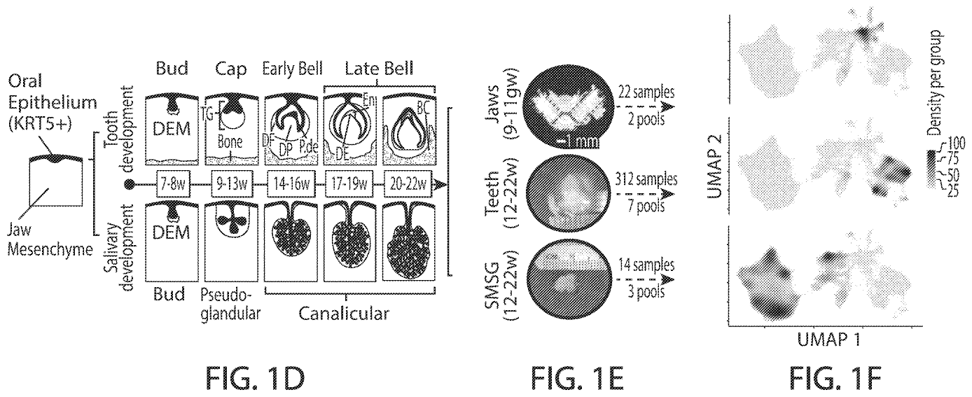

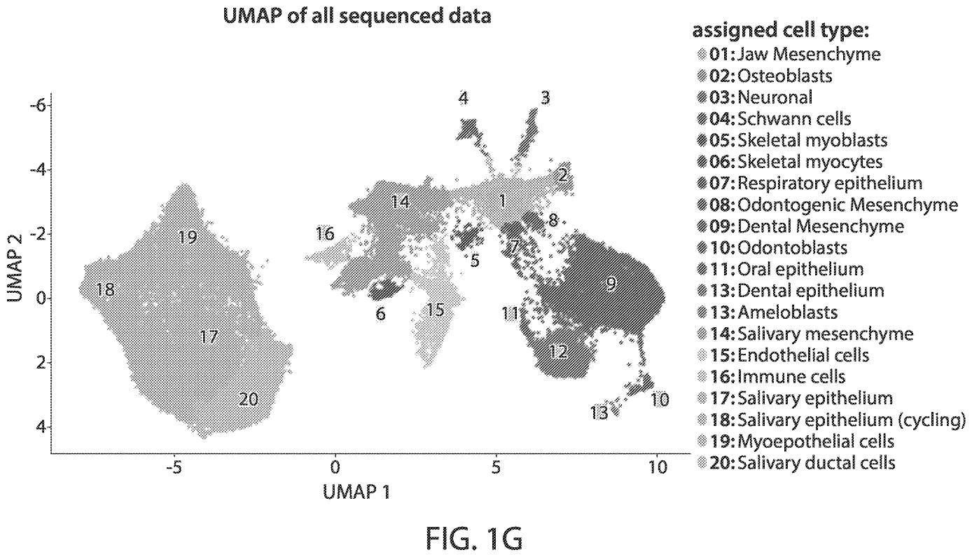

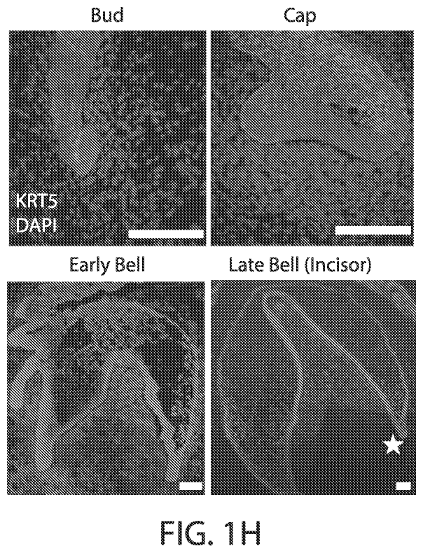

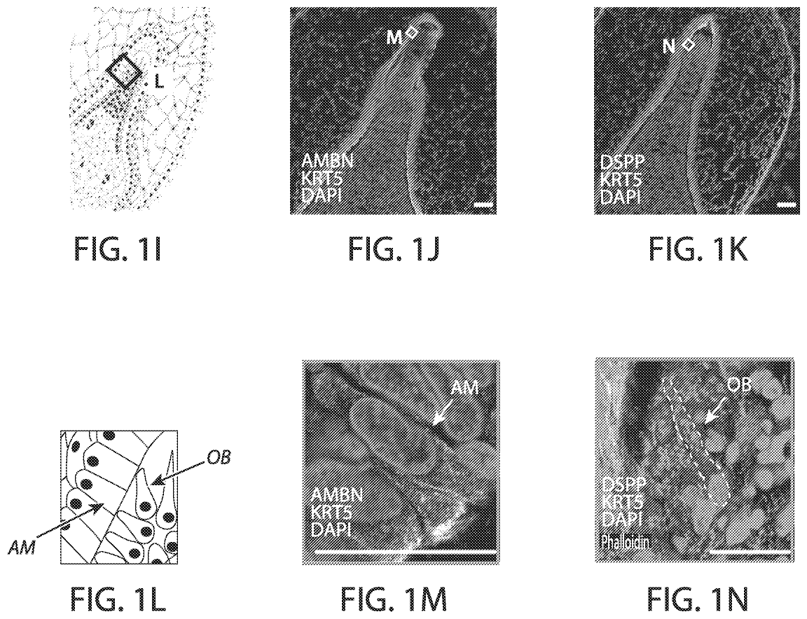

In humans, oral tissue development begins around 6 gw and starts as a thickening in the oral epithelium (de Paula et al., 2017; Jussila and Thesleff, 2012; Nanci and TenCate, 2018), giving rise to all primary teeth and salivary gland tissue. Individual teeth develop independently as an extension of the main dental lamina and progress through a series of morphological stages (bud, cap, & bell) within bony crypts of the jaws (Radlanski et al., 2016). Additionally, each developing tooth is surrounded by thick fibrous tissue called the dental follicle (Wise et al., 1998). The dental follicle and the tissue it contains comprise the toothgerm (Kardos and Hubbard, 1981) (FIG. 1A, ID). The oral epithelium will also give rise to the salivary glands (FIG. 1A, ID). Like teeth, salivary glands derive from the invagination of a thickened sheet of oral epithelium into the underlying mesenchyme, known as the initial bud stage (Cha, 2017) (FIG. 1A, 1D).

To better understand early oral differentiation and to dissect how the epithelial and mesenchymal cell lineages acquire the odontogenic competence, we analyzed the developmental gene expression profiles of human fetal stages by single-cell sequencing. Toothgerm and salivary gland samples were collected from five fetal age groups (FIGS. 1A-1B, FIGS. 1D-1E and FIGS. 9A-9C). These age groups represented the following developmental stages for tooth differentiation: the cap stage (9-13 gw), the early bell stage (14-16 gw), and the late bell stage (17-22 gw) (FIGS. 1A-1H) (Nanci and TenCate, 2018; Nelson, 2020). Submandibular salivary glands (SMSG) were also collected from three matched timepoints (12-13 gw, 14-16 gw, 17-19 gw) that cover the pseudo-glandular and canalicular stages for salivary gland development (Quirós-Terrón et al., 2019) (FIG. 1A, 1D).

Single-cell sequencing data of the tissue samples were analyzed using Monocle3 (Cao et al., 2019; Trapnell et al., 2014) and visualized in uniform manifold approximation and projection (UMAP) space (FIG. 1G). The distribution of the cells from each tissue origin was identified by using density plots based on tissue type (FIG. 1C, 1F) or by individual samples (FIG. 9D). Utilizing a graph-based clustering algorithm, 20 major clusters were annotated based on key marker genes (FIG. 1D, 1F; FIG. 9E) from PanglaoDB (Franzén et al., 2019). The major cell types in salivary gland samples include salivary mesenchyme, salivary epithelium, cycling salivary epithelium, myoepithelium, and ductal cells (FIGS. 1C-1D and FIG. 9E). In the jaw samples (9-11 gw) (FIGS. 1C-1D, 1 and FIG. 9E), we identified mesenchymal progenitors, osteoblasts, neuronal, Schwann cells, muscle, respiratory epithelium, otic epithelium, and oral epithelium (FIGS. 1C, 1F, 1G; and FIG. 9E). The major cell types in tooth samples include dental mesenchyme, epithelium, odontoblasts, and ameloblasts. The cell types observed in all samples include endothelial (Albelda et al., 1991; Jiang et al., 2016; Lampugnani et al., 1992) and immune (Böheim et al., 1987; Filion et al., 1990) cells. The salivary gland sci-RNA-seq data was previously analyzed in more detail (Ehnes et al., 2022). The present disclosure focuses on the gene expression and signaling pathways governing tooth development.

To confirm the timing of the tooth morphological stages, immunohistochemistry was performed on tissue sections. As expected, all the enamel organ derived tissues were visualized by KRT5 (FIG. 1E). There are two critical lineages in tooth development: odontoblasts and ameloblasts. These two cell types secrete the mineralized protective layers that cover the soft dental pulp, which contains the nerves and the nutrient-transporting blood vessels. Odontoblasts are ectomesenchyme-derived cells secreting the inner coverage for the pulp, called dentin, while ameloblasts are ectoderm-derived and secrete the outermost layer, enamel. In order to establish the expression of known odontoblast and ameloblast markers in our tissue, immunohistochemistry was performed on human fetal toothgerm at 20 gw using dentin sialophosphoprotein (DSPP) and ameloblastin (AMBN), respectively (FIGS. 1I-1N and FIGS. 12K-12Y). As expected, ameloblasts express AMBN in secretary vesicles (FIGS. 1L-1M); likewise, odontoblasts secrete DSPP (FIGS. 1L and 1N).

Spatial Localization of Sci-RNA-Seq Defined Clusters Identifies Subodontoblasts in Humans for the First Time and Suggests they Give Rise to Preodontoblasts in an Early Developmental Setting

To dissect the odontoblast lineage, the developing jaw mesenchyme, dental ectomesenchyme, and odontoblast cells was subset and embedded the data into a UMAP space (FIG. 1D and FIGS. 2A, 2J). This analysis yielded six transcriptionally unique clusters: dental papilla (DP), preodontoblast (POB), odontoblast (OB), subodontoblast (SOB), dental ectomesenchyme (DEM), and dental follicle (DF) (FIG. 2A, 2J). Cell types were identified by putative marker genes reported by monocle3 ‘top_marker’ function, which matches with recently identified odontoblast markers (Krivanek et al., 2020) (FIGS. 2B, 2K; FIGS. 10H-10I).

Furthermore, to evaluate the function of each cluster, gene ontology analysis was performed using ViSEAGO (Brionne et al., 2019), which uses data mining to establish semantic links between highly expressed genes in a given cluster. This analysis shows that DP and DEM are characterized by signaling, morphogenesis, and adhesion, supporting their role as precursor populations. In contrast, POB is characterized by their motility and migration, indicative of their alignment to the edge of the dental pulp. SOB indicates secretion, budding, projections, and branching, characteristics of a cell type sensing and influencing its environment, while OB shows GO-terms toward odontogenesis, tooth organization, and mineralization (FIGS. 2B, 2K).

To assess progenitor sources and cells' progression towards differentiation, pseudotime trajectory analysis was performed. This analysis indicates the presence of two progenitor sources within the developing dental mesenchyme: the DP that gives rise to POB and, subsequently, OB; and the DF that gives rise to SOB, which transition through a POB phase before giving rise to OB (FIG. 2L). Pseudotime analysis is supported by real-time density plots that show reduced progenitor type cell population density as the toothgerm develops, indicating fate commitment to OB lineage begins after 13 gw in human fetal development and is largely complete by 20 gw (FIGS. 2C, 2M). Broad expression of dental ectomesenchyme marker PRRX1 is observed in both the DEM and DP (FIG. 10J), supporting previous findings (Chai et al., 2000) that a shared cranial neural crest progenitor gives rise to both DP and DF. Thus, a simplified trajectory is proposed of both the odontoblast and dental follicle lineages (red and grey arrowheads in FIGS. 2H, 2N), with a shared PRRX1+ progenitor giving rise to both DEM and DP.

To localize the computationally identified clusters in human fetal tissue, RNAScope in situ hybridization was performed on toothgerms at early (13 gw, 80d) and late (19 gw, 117d) tooth development (FIGS. 2O and 2Q). After performing signal quantification per cell, the RNAScope images were converted into spatial datasets of single cells (FIGS. 2P and 2R; FIGS. 10L-10M). In agreement with the sci-RNA-seq data (FIG. 2B), dental mesenchyme-derived cell types display spatiotemporally specific expression patterns. At 13 gw (FIGS. 2O-2P), the dental pulp consists of DP with DEM localized to the apical portion. The presence of sparse DF cells within the dental pulp (black arrowheads in FIGS. 2P and 2R) supports the pseudotime trajectory suggesting DF as progenitors for SOB, and this commitment occurs prior to 13 gw. The developing toothgerm is surrounded by DF cells, a pattern that persists to late tooth development (19 gw) (FIGS. 2Q-2R). By 19 gw, it was observed that the dental pulp contains a mixed population of SOB and POB, with small contributions from DP, DEM, and OB at the incisal edge (FIGS. 2Q-2R). SOB was observed directly beneath the OB (black arrowhead, FIG. 2R) and, intermingled with POB at the pulpal periphery (red arrowhead, FIG. 2I). This finding supports the pseudotime trajectory (FIGS. 2F, 2G, 2L), indicating SOB can give rise to OB not only following injury, as seen previously in mouse models (Harada et al., 2008; Ruch et al., 1995) but also during normal human tooth development. SOB represents a small portion of the pulpal cell population (FIGS. 2Q-2R), suggesting that OBs are mainly derived from POB while SOB serves as a reserve with the capacity to differentiate to OB through a POB transitional state (FIGS. 2F, 2G, 2L; 2H, 2N, 21, and 2O-2T). This hypothesis is further strengthened by cell cycle analysis indicating SOB as a progenitor source of OB during normal tooth development, as this cell type has the highest proportion of cells in the G2 μM/S phase (FIG. 10K). Lineage tracing studies are necessary to validate this exciting finding in vivo and further dissect SOB's role in odontoblast development and repair.

### Sci-RNA-Seq and Spatial Localization Reveal Stage-Specific Support Cell Types and Cervical Loop Stem Cells for Ameloblast Differentiation

To further analyze the subtypes of the dental epithelium, oral epithelium, dental epithelium, and ameloblast clusters are subset (FIGS. 3A, 3C). The subset yielded 13 unique clusters that we identified by collating highly expressed cluster-specific genes (FIGS. 3B, 3D and 11C). Oral epithelium was identified (OE), dental epithelium (DE), enamel knot (EK), enamel epithelium (outer enamel epithelium/inner enamel epithelium, OEE/IEE), cervical loop (CL), inner and outer stratum intermedium (SII, SIO), inner and outer stellate reticulum (SRI and SRO), pre-ameloblasts (PA) and two AMBN expressing ameloblast clusters (early ‘eAM’ and secretory ‘sAM’; FIGS. 3A-3F). The identity of these clusters aligned with their likely real-time appearance as represented by a real-time distribution of cells (FIG. 11D). Moreover, GO analysis (FIG. 3B, 3D) indicated cell type-specific roles in tooth development in agreement with our annotations. For example, the OE cluster revealed proper stratified epithelium, including keratinization, keratinocyte differentiation, and cornification (Adams, 1976), while the DE shows epithelial organization and differentiation, indicative of its function in reorganizing to form the tooth bud (Ahtiainen et al., 2016).

To identify the developmental trajectory of the dental epithelial lineages, we performed pseudotime analysis (FIG. 3E) summarized by the simplified tree graphs (FIG. 3F, ameloblast trajectory with red arrows). The trajectory analysis suggests that the OE directly gives rise to DE. The DE then gives rise to the EK and SR lineages and the OEE lineage, which gives rise to SI, IEE/PA, and eAM/sAM. In order to validate our bioinformatic findings, we performed RNAScope in situ hybridization at multiple timepoints. We used combinations of cluster-specific markers identified by transcriptional analysis to map cells from each cluster in the fetal tissues (FIGS. 3K, 3L, and 3N; FIG. 11E). Computational pseudo-spatial mapping of these cells revealed the following insights on EK, support cells, and CL function (FIGS. 3K and 3M).

The EK is a structure that has previously been identified at various times in mouse tooth development and is thought to organize local cell proliferation for epithelial budding or folding during cap and bell stage transitions (Thesleff et al., 2001; Vaahtokari et al., 1996; Yu et al., 2020). Primary EK has been shown to appear at the time of the first folding of the toothgerm to form the cusp, followed by secondary EK formation for subsequent cusp development. A cluster of cells was identified consistent with EK in human fetal development. Real-time distribution showed that cells occupying this cluster appeared at 9-11 gw (early cap stage) and again at 14-16 gw (early bell stage) (FIGS. 3B, 3D; FIGS. 11B, 11D and FIGS. 11E; 3K), in line with the expected appearance of primary and secondary EK, respectively. EK are essential signaling centers in these early stages of tooth morphogenesis, playing a role in determining crown shape. Accordingly, GO terms identified in response to top gene expression associated with these clusters included morphogenesis and appendage development. These findings represent the first time this population has been identified at the transcriptional level and can lead to further understanding of the initiation of tooth morphogenesis and toothgerm type determination.

Multiple types of support tissues exist in the developing enamel organ. The SR are support cells with a star-shaped appearance in histological sections (Liu et al., 2016), which are thought to provide nutrients to and cushion the developing ameloblasts (Nanci and TenCate, 2018). Another support cell type, SI, is thought to support ameloblast differentiation (Liu et al., 2016)(FIGS. 3A-3D). Both types of support tissue were identified in human fetal tissues. Furthermore, the single-cell analysis expanded upon what is understood about these populations. Transcriptomic analysis revealed two subgroups of SR, inner SR (SRI) closer to the inner surface of the toothgerm and outer SR (SRO) (FIGS. 3A-3F; 3M and 11C). Analysis also identified, for the first time, two human SI sub-clusters that appear at 12 gw and persist to later development (FIGS. 3A-3B; 3H and 11C). Inner SI (SII) represents the cell layer closer to ameloblasts lineage, and outer SI (SIO) represents the parallel support cell types adjacent to SII. The SI lineage at the early bell stage consists of two layers of cells, SII and SIO, that lie near ameloblast lineage (IEE, PA, and AM) (17-19 gw) (FIGS. 3L-3M; 3P and 11G), creating a 3^{rd} previously unidentified stage-specific layer of cells (FIG. 3M bottom left enlarged box). Furthermore, at the late bell stage, PA differentiates into eAM and matures to sAM (17-19 gw) (FIG. 3M top enlarged box, FIG. 3P). These novel subgroups of support cells have precise signaling capacity to the specific, nearby epithelial cells in ameloblast lineage.

The enamel epithelium is the basal cell layer on the periphery of the tooth consisting of OEE, lining the outer side of the tooth, and IEE (Krivanek et al., 2020), lining the concave side of the folded tooth (Liu et al., 2016). As predicted, the transcriptional analysis revealed the presence of both of these populations, which was confirmed with RNAScope in situ hybridization (FIGS. 3A-3D and FIG. 3M bottom right enlarged box; FIG. 11G). sci-RNA-Seq and RNAScope analysis revealed that in the cap stage, the core cells of the enamel organ are the DE that will give rise to the signaling center EK and the OEE (12-13 gw) (FIGS. 3K and 3O). sci-RNA-Seq also revealed a small population of LGR6+ CL cells expressing markers previously reported in epithelial stem cells of the regenerating adult mouse incisor (Chang et al., 2013). These cells could be localized in human fetal tissue to the expected location of the CL, where the OEE and IEE meet (FIGS. 3N and 3P). During the early bell stage, OEE are the basal cells on the periphery of the tooth organ that gives rise to SI, CL, & PA lineages (17-19 gw) (FIGS. 3L; 3M and 3P). Importantly, the trajectory analysis predicts that the stem cells in CL can give rise to the ameloblast lineage. These data suggest that CL has a stage-specific role; while CL in humans is traditionally thought to be involved in later root development, our data suggest that during the early stage of fetal development, CL has a vital function in generating ameloblast lineage as the tooth crown expands

Sci-RNA-Seq Reveals Spatio-Temporal Expression Patterns of Critical Signaling Pathways in Ameloblasts and Facilitates the Development of Human iPSC-Derived Ameloblasts (iAM) In Vitro

To understand the signaling pathways involved in ameloblast differentiation, we compiled a comprehensive multiplexed analysis pipeline based on ligand-receptor interactions and downstream transcriptional activity (FIG. 14A). Briefly, a talklr (Wang, 2020) R package was used to identify specific ligand-receptor communications between the cell types at each developmental time point. DEsingle (Miao et al., 2018) and scMLnet (Cheng et al., 2021) programs were used to evaluate the downstream signaling activity by establishing multilayer networks between ligands and receptors and between transcription factors and their differentially expressed targets. Finally, activity scores were assigned to each pathway, which represent a percentage (0-100%) of the overall activity for all pathways included in the analysis.

Following our analysis pipeline, the pathway activities were evaluated between each stage in the ameloblast developmental trajectory and identified the most active pathways with specified ligands in each step (FIGS. 5A-5B; FIGS. 14A-14C). To identify the main sources of the secreted ligands for each active pathway, talklr was used (Wang, 2020), with the ligand gene expression level analysis as a secondary validation (FIGS. 5C-5D and FIGS. 14B-14C). These data revealed that during the transition from OE to DE, the BMP, ACTIVIN and noncanonical WNT (ncWNT) signals are secreted from the dental mesenchyme, while the canonical WNT ligands are secreted from within the OE. Similarly, during DE to OEE, the signaling ligands are secreted from within DE and EK (FIGS. 14B-14C). Meanwhile, the BMP and FGF ligands are mainly secreted from the surrounding dental mesenchyme. During the transition from OEE to IEE, the dental mesenchyme, which is now condensed as the dental papilla, mainly affects the ameloblast lineage by secreting BMP. Perhaps more interestingly, ligands for the prominent TGFβ pathway are mainly secreted from the support cells SRI, highlighting the importance of the spatio-temporal support cells in ameloblast differentiation. Other support types associated with stage-specific signaling behavior include SII ncWNT/HH/EGF, while SIO secretes FGF to support the last stages of ameloblast development and maturation. Additionally, mesoderm-derived POB and OB showed significant interaction with epithelial clusters; both secreted FGF and BMP ligands at PA during the PA to eAM transition or the transition to sAM. During ameloblast maturation, WNT ligands are mainly secreted from within eAM (FIG. 5D).

Based on the signaling pathway prediction analysis, it was found that BMP, ncWNT and ACTIVIN pathways are most active during OE to DE transition. However, ncWNT and ACTIVIN pathways down-regulate during DE to OEE transition when FGF and WNT pathways become more prominent. During the OEE to IEE stage transition, BMP is the most active, followed by WNT and ACTIVIN pathways. Meanwhile, in IEE to PA stage, mostly WNT (40% including canonical and non-canonical), HH (17%), and EGF (10%) become more active. In PA to eAM transition, the HH is the most active at (35%), followed by WNT, BMP and TGFβ. To analyze the maturation stage, the pathway activities were evaluated between eAM and sAM clusters and found that FGF, WNT and EGF signaling are involved in ameloblast maturation (FIGS. 5A-5B and FIGS. 14B and 14D). The analysis of the stages of ameloblast development reveals a critical function for support cells, SI, SR and mesoderm signaling: BMP and ACTIVIN from mesenchyme are involved in the transition from OE to DE, ncWNT from DE, and support cells and BMP again from mesenchyme in the transition from DE to OEE, and from OEE to IEE. Similarly, IEE to PA differentiation utilizes specific accompanying SII to secrete ncWNT/EGF and WNT from SRI (FIGS. 5A-5D and FIGS. 14B-14E). In last stages of ameloblast differentiation, from PA to eAM, PA and SII secrete HH ligands, while SRI and SIO secrete TGFβ. At the final maturation stage, from eAM to sAM, SII secretes EGF & SIO secretes FGF. Interestingly, WNT activity in the transition of OEE to IEE (FIGS. 14B-14E) can be linked to the emergence of SP6 expression in IEE in the junction of the cervical loop (FIG. 5E). WNT pathway has been suggested to work upstream of the expression of the transcription factor SP6 (Aurrekoetxea et al., 2016; Haro et al., 2014; Ibarretxe et al., 2012), which in turn was found to interact with AMBN/AMELX promoters (Rhodes et al., 2021) (FIGS. 5E-5J). Additionally, it was found that SP6 is mostly localized in the cytoplasm of the early stages IEE/PA. However, in later stages, SP6 is localized to the nuclei coinciding with AMBN expression in eAM/sAM (FIG. 5H). These data support the hypothesis that SP6 expression is induced by WNT pathway already in IEE transition stage, but becomes functional in eAM stage when it translocates to the nucleus and induces AMBN expression. Future loss-of-function analysis is required to test this hypothesis. Together these data suggest that WNT, TGFβ, HH, FGF, and BMP pathways are the top active pathways in ameloblast development compared to all 25 pathways included in the analysis.

The inferred signaling pathways were utilized from the sci-RNA-seq data (FIGS. 5A-5D) to develop a novel in vitro differentiation protocol that recapitulated the early stages of human ameloblast development from hiPSCs (iAM differentiation; FIGS. 5A-5B). A protocol was optimized to differentiate iPSC into OE (Ochiai et al., 2015; Suga et al., 2011; Tanaka et al., 2018). At day 10 of differentiation, the OE markers were upregulated, while pluripotency markers were downregulated, and neuroepithelial and early mesodermal markers remained unchanged (FIG. 7B; FIG. 15B). To differentiate the OE cells into an early stage ameloblasts, the main pathways were active and identified (FIGS. 5A-5B) from the OE stage to the PA stage (BMP4, TGFβ1, WNT/CHIR99021, EGF, and HH/SAG pathways in the order of their activity during differentiation (FIGS. 7A; 7C). To transiently inhibit the BMP pathway, the small molecule LDN was used. This differentiation procedure resulted in marked epithelial morphological changes and a high expression of the ameloblast early marker AMBN at day 16 in the differentiation (FIG. 7B; FIGS. 15A-15B, 15I), indicative of the early ameloblast differentiation stage.

To dissect which pathways were essential for the differentiation from day 10 to day 16, the process of elimination was used (FIG. 7C). When removing EGF, SAG, BMP4, or TGFβ1 independently, it was found that the expression of AMBN is significantly reduced to less than half compared to when all factors are present. However, removing GSKi completely abolished AMBN expression, suggesting that WNT signaling is a master regulator upstream to other pathways (FIG. 7C). The pathway prediction pipeline also suggested that the FGF pathway is heavily involved; however, adding bFGF had no significant effect on AMBN expression (FIG. 7C). It was hypothesized that the cells in culture secrete enough FGF ligands to saturate the receptors, and any exogenous ligands would have minimal effects. To test this hypothesis, a computationally designed protein was used, FGFR2 mini-binder (FGFR-mb), that specifically binds and inhibits the activity of the FGFR2 (Cao et al., 2022). Adding the FGFR2-binder almost totally abolished the expression of AMBN, which shows that the FGFR pathway is indeed required for ameloblast differentiation (FIG. 7C). This marks the importance of highly specific AI-designed mini-proteins in analyzing the requirement of signaling pathways in differentiation. It is plausible that temporal preciseness and high penetrance, combined with specificity of designed mini-binders to their targets may partially out-compete in the future genetic perturbations of signaling pathway in iPSC derived differentiation paradigms.

To analyze the efficiency of the differentiation, sci-RNA-seq was performed on Day 10 and Day 16 of iPSC derived ameloblast differentiation (day 10-OE and day 16-Early-ameloblasts) and compared the gene expression data to the fetal tissue gene expression data. The initial clustering and trajectory analysis indicated three major clusters at day 10 and six clusters at day 16 (FIG. 15J-15K). Sequencing revealed a significant overlap between human fetal and iPSC-derived ameloblasts in 2D culture. A survey of relevant markers to the dental epithelium (FIGS. 15K-15M) showed the kinetics of their differential expression across the proposed trajectory (FIGS. 15J-15K). Utilizing the markers for the oral/dental epithelial progenitors (Sun et al., 2016; Yu et al., 2020), enamel epithelium (Nakamura et al., 2017), and ameloblasts (Seidel et al., 2010), all the differentiated cell types were identified (FIG. 15L). For a better comparison between the in vivo and in vitro datasets, the projection method was used in Seurat 4.0 and the integration method in LIGER software packages (Hao et al., 2021; Welch et al., 2019) to overlay the datasets. The dataset was converted from Monocle3 format to Seurat format; then, the projection was performed over the UMAP of the fetal dental epithelium lineage. Lastly, the projected cells were classified using graph-based clustering. A small proportion of the cells in the day 16 sample were OE-like, DE-like, SR-like, and SI-like. However, the majority (60%) were PA and AM-like, indicating that most of the differentiated cells are directed toward the ameloblast lineage (FIG. 5F). River plot analysis was performed using LIGER to show the relationship between the annotated clusters from the fetal dental epithelial lineage and the in vitro day 16 differentiation clusters that share the same space in LIGER joint clusters (FIG. 15L), which allows label matching for the unannotated clusters in the differentiation (FIG. 5G). The fetal OE cluster matched cluster 1 (d16_1; FIG. 15K) in in vitro differentiation; the DE, SR, and OEE from in vivo samples mainly matched cluster 2 (d16_2) and SI matching cluster 4 (d16_4). The pre-ameloblast and ameloblast clusters matched clusters 5 and 6 (d16_5, d16_6), respectively, which represent 47% of total cells (FIG. 5E). Finally, the functionality of the iAM was analyzed by analyzing the number of cells in day 16 differentiated samples that produced AMBN, the product secreted by ameloblasts. Notably, 25% of the cells in 16 days of differentiation can produce and, in some cases, secrete AMBN protein (FIG. 15I). This analysis suggests that the iAMs share similarities with fetal pre-ameloblasts and ameloblasts, demonstrating that the described 2D procedure can generate early differentiated ameloblasts.

### 3D Enamel Organoids Show Mineralization and Ameloblastin, Amelogenin, and Enamelin Secretion

To further characterize iAM and evaluate their capacity to mature in vivo, the differentiated cells (day 16, 2D) were injected intramuscularly into adult SCID mice and allowed the injected cells to develop for 8 weeks (FIG. 8A). The injected region was identified by human nuclear antigen staining (FIG. 8B). The maturation stage of the iAM cells was analyzed in the subsequent serial sections by definitive ameloblast markers: AMELX, AMBN, DSPP, KRT14, and by its calcification capacity. Importantly, the identified iAM cells were significantly more mature (FIG. 8B; 8E and FIG. 16A-16G), showing that the iPSC derived iAM cells have a capacity to develop to a more mature AM stage. The highly elongated morphology of these cells (FIG. 8D) suggests that they have developed into so-called secretory stage AM (sAM) that characteristically consists of tall columnar cells that express amelogenin (AMELX) and ameloblastin (AMBN) and produce mineralization. Accordingly, iAM capacity to produce calcified material was identified via Alizarin red and Von Kossa staining (FIGS. 8E and 16G).

Since close contact between AM and OB is critical for tooth development, we proceeded towards developing an organoid model of the two cell types. An organoid model was first developed of polarized AM. To generate cells expressing AMBN in a culture with apical-basal polarization, similar to ameloblasts in vivo, the cells were grown in suspension to form spheroids (FIG. 8F). Immunofluorescence staining was performed for SP6, AMBN (FIG. 8G), and ZO1, DSPP (FIG. 8H). It was observed that the transcription factor SP6 is expressed in all the differentiated cells and is exclusively localized to the nucleus. The induced ameloblasts show apical-basal polarity and secrete AMBN to the apical surface. As seen in in vivo AM, the nucleus is located towards the basal side of the cell (FIGS. 8H-8I). Early ameloblasts are known to transiently express DSPP during development (FIG. 1K; FIG. 3L and FIGS. 13X-13Y); it was noticed that DSPP expression is also localized to the apical side of the induced cells. Moreover, the tight junction protein ZO1 marks the apical side of these iAM cells (FIGS. 8H-8I). The iAM in the organoids appear as tall columnar cells polarized toward a central lumen (FIG. 8I).

The induced ameloblast organoids were co-cultured with primary human dental pulp stem cells (DPSCs) to assess the interaction level between the two cell types and the effects on ameloblast maturation. The simple coculture in suspension can induce AMELX in iAM organoids and DSPP in the odontoblast organoids, as observed in the developing human tooth (FIGS. 13A-13J); as well as induction of calcified matrix (FIGS. 16H-16K). After confirming that iAM can mature in the presence of OB/DPSCs, the following experiment was designed to coculture the cells in a layered approach. The DPSCs were plated in the bottom of a flat bottom plate and then embedded iAM organoids in a Matrigel layer above the DPSCs (FIG. 8J). The co-culture media contained iAM and odontogenic media at 1:1 ratio, with calcein in addition to detect calcification. Through 3D reconstructed confocal images, it was observed that iAM are associated with calcein, demonstrating the capacity of iAM to produce mineralization/calcification (FIG. 8K). Furthermore, the co-cultured iAM expressed ENAM and AMELX (FIG. 8L) and reverted their polarity towards the differentiating OB (FIG. 8L-8N). This 3D organoid, therefore, mimics the normal cell-to-cell interface observed in developing tooth where the enamel proteins are secreted towards the OB and sets the stage towards developing human tooth organoids in a dish.

### DISCUSSION

Functional ameloblasts and odontoblasts are two critical cell types secreting the protective tooth coverings, enamel, and dentin, that are required to generate the functional structure of teeth. Ameloblasts do not exist in adult oral structures, making enamel regeneration impossible; however, dentin-secreting odontoblasts are critical for the regeneration of adult teeth. While previous morphological studies have suggested that two cell types can give rise to odontoblasts, the developmental lineages and molecular characterization of this process were not understood. Single-cell sequencing was generated and utilized to identify the cell types in the developing human tooth and their molecular interactions across several developmental stages. The major cell types were identified in human oral development that derive from the jaw tissue and give rise to teeth and salivary glands. Significantly, the presence of subodontoblast cells was shown for the first time in human tissue. Additionally, mesenchymal and epithelial odontogenic progenitors were further characterized and revealed potential developmental trajectories that lead to odontoblasts and ameloblasts, respectively. Importantly, novel human support cell types were identified that significantly and precisely promote the differentiation of ameloblasts. Analyzing the signaling interaction in the ameloblast trajectory allowed for the prediction the signaling molecules needed to recapitulate ameloblast development in vitro. Utilizing these findings, a novel differentiation protocol was developed to drive the differentiation of iPSCs toward early ameloblasts (iAM). Their identity was successfully verified by comparing the expression profile of the in vitro generated ameloblast lineage to the in vivo fetal counterpart. Finally, this information was used to develop an enamel organoid that expresses mature ameloblast markers and secretes mineralized calcium.

The sci-RNA-seq data revealed novel transcriptionally defined subgroups of cells in both the epithelial and mesenchymal lineages. The analyses identified 13 subclusters of cell types in the dental epithelial lineage, of which SRI, SRO, SIO, and SII are novel types of support cells in human tooth development. The newly identified support cell type, SRI, produces a TGFβ ligand at an early stage of tooth development to aid in the differentiation of IEE to PA. While SII secrete EGF and SIO secret FGF ligands at later stages to aid in the maturation of AM. The data have amended the detail with which the understanding of how support cells contribute to the patterning and development of ameloblasts. The analysis also revealed a previously undescribed role for SOB in the mesenchymal lineage. It was shown that while human OB are derived from POB, surprisingly, the developing human fetus has two potential sources that generate POB: DP or DF. Both precursor stages are strictly found in early fetal tissue; after 20 gw, these precursors are largely absent in the dental pulp. A portion of DF cells differentiates to SOBs that have a more permanent, but previously unidentified function in tooth development. This novel SOB cell type is characterized on a molecular level and shown that they have characteristics of cells that sense and influence their environment, supporting the idea that these cells can sense the need to regenerate the lost OB population (Harada et al., 2008). Furthermore, a well-known disease gene in tooth development (Duverger and Morasso, 2018; Duverger et al., 2012), DLX3, is a key marker for SOB, calling for further analysis of DLX3 function in this critical preserved group of cells in a disease-in-a-dish approach.

For the first time in human tooth development, the studies have revealed, in extreme detail, the signaling pathways that govern each transition between cell identity. Previous studies of hypodontia and tooth agenesis have shown that disruption of WNT, BMP, and FGF signals results in defective tooth development. However, the detail with which the study has revealed the role of these pathways at various points in development may more mechanistically explain how defects in these pathways lead to tooth loss or tooth agenesis. For example, studies have shown that mutations in BMP4 correlated to tooth agenesis (Yu et al., 2019a). The analysis showed that BMP4 signaling is critical during the early stages of both the OE to DE and DE to OEE transition, suggesting that loss of BMP4 may lead to agenesis by disrupting these transitions. In other studies, disruption of FGF signaling leads to enamel irregularities (Marangoni et al., 2019). Using AI-based protein design, it is revealed that FGF signaling is essential at the point of ameloblast maturation, suggesting that these irregularities are a result of failure of ameloblasts to mature. While some studies have focused on the role of a single signaling pathway, many others have highlighted the importance of crosstalk between pathways in tooth development and maintenance (Liu et al., 2020; Malik et al., 2018; Yu et al., 2019b). The predictive pathway analysis highlights not only the primary pathway responsible for each stage but also ranks the other pathways involved. Overall, the study will facilitate the investigation into both previously identified and yet undescribed crosstalk in driving forward development. The detailed analysis provided in this study will facilitate more detailed and informed studies on degenerative dental diseases and can lead to developing more effective ways to mitigate or reverse tooth loss. Furthermore, the work with AI-designed, de novo receptor mini-binders that specifically bind and inhibit target receptor signaling (Cao et al., 2022) reveals a novel, highly simplified method to identify the exact stage of a specific signaling pathway required in the differentiation process. The method described in this study using the de novo FGFR-mb to unravel the FGFR pathway requirement in ameloblast maturation will be generally applicable and specific to any signaling pathway analyzed in the differentiation of normal and disease organoids.

Ameloblasts secrete the most mineralized and highly vulnerable layer in the human tooth. However, this cell type, and hence enamel regeneration, is absent in adult humans, presenting an impasse for progress in human regenerative dentistry. The studies have revealed multiple new potential avenues through which further study could overcome this. First, the studies present the first single-cell analysis and in vivo localization of the cervical loop in human fetal teeth. The cervical loop is part of the enamel organ in the developing tooth located where the OEE and the IEE join. It has been extensively studied in the mouse, most often in the mouse incisor. However, unlike the human tooth, the mouse incisor grows continuously, with the cervical loop serving as a reservoir of stem cells that contribute to that consistent growth. Therefore, it is necessary to understand the function and contribution of the cervical loop in human tissue. Classically, the cervical loop is known to give rise to Hertwig's Epithelial Root Sheath, which initiates root formation. Intriguingly, the analysis revealed a role for the CL in giving rise to human ameloblasts in early tooth development, as the crown expands before the root begins to form.

Finally, the present work characterized the molecular basis for human ameloblast differentiation. This knowledge has been used to develop an assay for differentiating human iPSC-derived ameloblasts in a dish (iAM). Comparing fetal data to the iAM differentiation suggests that iAM shares high similarity with fetal pre-ameloblasts and early ameloblasts. Further, iAM can reach the secretory stage since Ameloblastin protein production and secretion is observed in these cells. In addition, the iAM cells showed a significant increase in maturation, including calcifications, when tested in vivo. Upon co-culturing iAM and OB lineage we observed the iAM reverting their polarity and apical secretion of enamel proteins toward the OB lineage cells. Hence, it is argued to have developed a chemically defined serum-free differentiation protocol to generate human dental epithelium, and their subsequent differentiation into enamel organ-like 3D organoids. This developed organoid can contribute to dental therapeutic approaches.

The first human single-cell tooth development atlas described here paves the way toward successful human regenerative dentistry. The molecular analysis and in vitro ameloblast differentiation protocol allow further dissection of diseases such as Amelogenesis Imperfecta that can guide the field toward therapeutic approaches.

### Materials and Methods

### Tissue Collection and Dissection

This study is approved by the Institutional Review Boards (IRB) at University of Washington for the use of human fetal tissues: BDRL (CR000000131) and Ruohola-Baker Laboratory (STUDY00005235). Fetal craniofacial tissues were collected from Birth Defect Research Laboratory (BDRL), University of Washington, and transferred to Ruohola-Baker laboratory submerged in Hank's Balanced Salt Solution (HBSS) media (Gibco, #14025092) on ice. Toothgerms and salivary glands were dissected in cold RNase free Phosphate-Buffered Saline (PBS) (Invitrogen, #AM9624) within six hours from the initial dissection at BDRL. To extract the toothgerms, a vertical cut was made at the midline of the upper/lower jaw for orientation, then a horizontal cut was made from the right side of the midline along the top of the alveolar ridge to expose one toothgerm at a time. The first two toothgerms from the midline were the incisors, the next toothgerm was the canine, and the last two toothgerms were the molars. The same procedure was followed to extract toothgerms on the left side of the jaw. The submandibular salivary glands were harvested from the distal end of the lower jaw. The toothgerms from 9 to 11 weeks old were too small for dissection and not useable for sequencing; therefore, these jaws were cut into two posterior sections and one anterior section to separate molars from the incisors and canines at these timepoints. The extracted tissues were transferred into an Eppendorf tube and snap frozen using liquid nitrogen. The frozen samples were stored at −80° C. until nuclei extraction.

### Nuclei Extraction

Frozen tissues were carefully transferred to a stack of chilled aluminum foil kept on dry ice to prevent thawing. The folded foil encapsulating the tissues were placed on a block of dry ice and the foil was pounded with a pestle to pulverize the tissues into powder. 1 mL of lysis buffer that contains nuclei buffer (10 mM Tris-HCl, 10 mM NaCl, 3 mM MgCl₂, pH 7.4), 0.1% IGEPAL CA-630, 1% SUPERase In RNase inhibitor (20 U/μL, Thermo), and 1% BSA (20 mg/mL, NEB) were added onto the tissue powder and transferred to a 1.5 mL tubes. Samples were incubated in the lysis buffer for 1 hour on ice. The samples were pipetted up and down with pre-cut 1000 uL pipette tip to disassociate the tissue further. The dissociated tissues were passed through 70 um cell strainers (Corning) into a 50 mL conical tube. The strainers were rinsed with lysis buffer to minimize nuclei loss. The samples were centrifuged to pellet the nuclei at 500 g for 5 minutes at 4° C. and the supernatant was discarded. The samples resuspended again in 1 ml lysis buffer, transferred into new 15 mL tubes, pelleted again and the supernatant was discarded. The pellets were resuspended in 50 ul of nuclei buffer, and 5 mL of 4% Paraformaldehyde (PFA) (EMS) diluted in RNase free PBS, was added to fix the nuclei for 15 minutes on ice. The tubes were flicked gently every 5 minutes to reduce clumping of nuclei. The fixed nuclei were pelleted at 500 g for 3 minutes at 4° C. and the PFA waste was discarded. The pelleted nuclei washed in nuclei wash buffer (cell lysis buffer without IGEPAL) and then centrifuged again at 500 g for 5 minutes 4° C., and the supernatant was discarded. Finally, the pellets were resuspended again in nuclei wash buffer and then flash-frozen in liquid nitrogen before storing in −80° C.

For nuclei extraction from the differentiation culture, the cells were treated with StemPro Accutase (Thermo, #A1110501) for 7 min to detach the cells and transfer them into 15 mL tube, then incubated in trypsin (Thermo, #25300054) for another 7 min to prevent re-clumping. The cells were span down to remove trypsin after inactivation with more media. The pellet was treated with nuclei lysis buffer and the same steps for nuclei extraction protocol were followed.

### Sci-RNA-seq

Single-cell combinatorial-indexing RNA-sequencing (sci-RNA-seq) protocol is described previously (Cao et al., 2019). sci-RNA-seq relies on the following steps, (i) thawed nuclei were permeabilized with 0.2% TritonX-100 (Sigma, #T9284) (in nuclei wash buffer) for 3 min on ice, and briefly sonicated to reduce nuclei clumping; (ii) nuclei distributed across 96-well plates; (iii) A first molecular index is introduced to the mRNA of cells within each well, with in situ reverse transcription (RT) incorporating the unique molecular identifiers (UMIs); (iv) All cells were pooled and redistributed to multiple 96-well plates in limiting numbers (e.g., 10 to 100 per well) and a second molecular index is introduced by hairpin ligation; (v) Second strand synthesis, tagmentation, purification and indexed PCR; (vi) Library purification and sequencing is performed.

All libraries were sequenced on one NovaSeq platform (Illumina). Base calls, downstream sequence processing and single-cell digital-expression matrix generation steps were similar to what was described in sci-RNA-seq3 paper (Cao et al., 2019). STAR (Dobin et al., 2013) v.2.5.2b54 aligner used with default settings and gene annotations (GRCh38-primary-assembly, gencode.v27). Uniquely mapping reads were extracted, and duplicates were removed using the UMI sequence, reverse transcription index, hairpin ligation adaptor index and read 2 end-coordinate (that is, reads with identical UMI, reverse transcription index, ligation adaptor index and tagmentation site were considered duplicates).

### Data Analysis

All low-quality reads were removed from the data (including jaws, toothgerms and salivary glands samples from all time points) by setting UMI cutoff to greater than 200 and removing all mitochondrial reads (QC table: FIG. 9F). Following Monocle3 workflow (Cao et al., 2019; Qiu et al., 2017; Trapnell et al., 2014), data underwent normalization by size factor, preprocessing, dimension reduction (UMAP algorithm (McInnes et al., 2018)), and unsupervised graph-based clustering analysis (Leiden Algorithm (Levine et al., 2015; Traag et al., 2019)). Certain clusters from the initial analysis were selected for further sub-clustering, and the previous analysis repeated. Pseudotime analysis also was done with Monocle3 following the default workflow, which include learning the graph, ordering the cells, and plotting the trajectory over UMAP. Mutual nearest neighbors (MNNs) algorithm (Haghverdi et al., 2018) was used for batch effect correction only between day 10 differentiation sample and day 16 sample. PanglaoDB (Franzén et al., 2019), a curated single-cell gene expression database was utilized to explore the consensus of cell type markers used across publicly available single-cell datasets.

### Top Marker Genes

Each dataset or subset was analyzed with monocle's top maker function to find potential marker genes. All non-protein coding genes, ribosomal and mitochondrial genes were excluded from the input genes, and only the top 100 genes sorter by marker score were included in the results.

### Heatmap and GO-Terms Enrichment

ComplexHeatmap package (Gu et al., 2016) was used to generate custom heatmaps that integrate GO-terms per clusters. ViSEAGO package (Brionne et al., 2019) used to generate the GO-terms, and simplifyEnrichment package (Gu and Hübschmann, 2021) used to extract keywords from the top 100 GO-terms (by p value) per cluster. The top 50 marker genes in each cluster were utilized as the input for ViSEAGO. The keywords generated by simplifyEnrichment, were filtered to eliminate redundant and irrelevant words, and only the very top words are displayed on the heatmap.

### Pseudotime Analysis

Pseudotime analysis was done using monocle3 and the density outline of each time point were overlayed on the UMAP graph, to give a better indication of the temporal presence of each cluster.

### Top Pathway Analysis

The stages of ameloblast development were analyzed as identified in (FIG. 3F). OEE and CL were combined as one OEE cluster to increase the statistical power (FIGS. 6A, 7A). To analyze those stages in a thorough and reproducible manner, a comprehensive analysis pipeline was compiled that evaluate pathway activity based on ligand receptor interaction and downstream activity. The workflow for our analysis is shown in (FIG. 14A). The first step in the analysis is selecting the appropriate input for each stage of the differentiation to be analyzed. At each stage, the progenitor cells and the target cell type to be differentiated into were considered, as well as all the support cell types that are present in the same stage and that are likely to send the signals. The second step is to analyze all the potential ligand-receptor interactions between the selected cell types, but only focus on in-coming interactions toward the progenitor cells of interest. For this part of the analysis, a software was used, talklr package (Wang, 2020), which uses an information-theoretic approach to identify and rank ligand-receptor interactions with high cell type-specificity. talklr output was further filtered by selecting those ligand-receptor pairs that fall within the major signaling pathway of interest (TGFβ, BMP, GDF, GDNF, NODAL, ACTIVIN, WNT, ncWNT, EGF, NRG, FGF, PDGF, VEGF, IGF, INSULIN, HH, EDA, NGF, NT, FLT3, HGF, ROBO, NOTCH, NRXN, OCLN). The third step of the workflow is to obtain the differentially expressed genes (DEGs) between the progenitor cells of interest and their differentiated cell type. This set of genes can be used to evaluate the downstream activity and can be linked to specific ligand-receptor pairs. DEsingle package was used (Miao et al., 2018) with FDR threshold set to 0.1 to obtain DEGs. The top marker genes for the progenitor cells were also excluded from DEGs in this analysis, to ensure more weight is given to the differentiated cell type. The fourth step is to generate a multilayer network that models the upstream interactions (obtained from step #2) and the downstream interactions that includes transcription factors (TF) and their target genes (DEGs obtained from step #3). The R package scMLnet was used (Cheng et al., 2021) to generate the multilayered network interactions that consists of a top layer for ligands, a layer for receptors, a layer for TFs and a layer for TF-targets. The fifth step is to implement a scoring system to evaluate the connectivity of each part of the multilayered network obtained from previous step, to determine which path is more probably active. Fold-change values were assigned (obtained in step #3) to target genes at the lowest level. At next level, the TF layer, the mean values were assigned to all the connected TF-targets to each TF. Normalization of the scores to the interaction database depth is done after each step, to ensure the scores remain comparable with each category of interactions. At the receptor layer, the sum of the values was calculated for all the connected TFs to each receptor. At the ligand layer, the sum of the values was calculated for all the connected receptors to each ligand. And finally, all ligands that fall within the same pathway family are aggregated together. The sixth step of our pipeline is to rank pathways based on the percentage of activity compared to the overall combined activity scores of all pathways evaluated in this analysis. The results indicate the most active pathways or the most active ligands that are key drivers of the differentiation at a specific stage of development (FIGS. 6A, 7A).

### Differential Expression

DEsingle package (Miao et al., 2018) was used to calculate differential expression (DE) between clusters. DEsingle was designed for single-cell RNA sequencing, and it employs Zero-Inflated Negative Binomial model to estimate the proportion of real and dropout zeros. The cutoff for DE genes were set to include genes with False Discovery Rate (FDR)<0.1 and more than twofold change.

### Multilayer Network Analysis

To generate a multilayer network that models the upstream interactions and the downstream interactions that includes transcription factors (TF) and their target genes, the R package scMLnet was used (Cheng et al., 2021). A custom wrapper code was developed to integrate talklr and DEsingle results with scMLnet.

### Signaling Interaction

In the study, talklr package was used (Wang, 2020) to identify ligand-receptor interaction changes between two adjacent tooth developmental stages. talklr uses an information-theoretic approach to identify ligand-receptor interactions with high cell type-specificity. Ligand-receptor interaction score is defined as Li*Rj, the product of expression levels for the ligand in cell type i and the receptor in cell type j. We normalize interaction scores by dividing Li*Rj with the sum of interaction scores across all n2 cell-cell interactions. talklr uses the Kullback-Leibler divergence to quantify how much the observed interaction score distribution differs from the reference distribution. The reference distribution is the equi-probable distribution where every possible interaction has

$$\frac{1}{{n}^{2}}$$

probability, when the aim is to identify cell type-specific ligand-receptor pairs in a single condition-Compared to existing methods such as cellPhoneDB (Efremova et al., 2020) or singleCellSignalR (Cabello-Aguilar et al., 2020) the unique strength of talklr is that it can automatically uncover changes in ligand-receptor re-wiring between two conditions (e.g. different time points, disease vs. normal), where the reference distribution is the observed interaction scores in the baseline condition. The parameters used were 0.001 for expression threshold, which was determined by calculating the level of expression of the 20th quantile of the aggregated clusters, and 1e-06 for the pseudo-count value which was determined by the minimum averaged expression value in the set. The interactions were considered among the top 100 ligand-receptor pairs returned by talklr, and were further prioritized by selecting those that are known to be from physically proximal cell types.

### Datasets Projection Analysis

Seurat 4.0 package (Hao et al., 2021) was used to project the invitro differentiation sample into the UMAP space of fetal ameloblasts sample. The dataset in monocle object format that contains the precomputed PCA and UMAP was converted into Seurat object. The projection was done with the default parameters. Graph-based clustering was performed on the projected data by calculating the nearest neighbor cluster center of the fetal sample. Package ‘networkD3’ (Allaire et al., 2017) was used to create the river plot showing the proportions of the classified cells.

### Datasets Integration Analysis

LIGER package (Welch et al., 2019) was used to integrate the fetal dental epithelium lineage dataset with the differentiation datasets to facilitate the cell type label transfer between the sets. The following integration parameters were used: k=25, lambda=10, and these settings were determined by utilizing the built-in function that suggest the best values that suit our datasets. For the river plot generation, the minimum fraction of the branching streams was set to 0.25, and the minimum number of cells set to 50. Clusters that have no out- or ingoing connection were eliminated from the graph for clarity.

### RNA Fluorescence In Situ Hybridization (FISH) and Analysis

A 12-probe RNAScope HiPlex assay (Advanced Cell Diagnostics, Inc.) including probes against 13 transcripts differentially expressed between cell type clusters in mesenchyme- and epithelial-derived lineages were selected to distinguish cell populations: VWDE, SALL1, FGF4, IGFBP5, FGF10, PRRX1, FBN2, ENAM, PCDH7, SOX5, KRT5, and either DSPP or LGR6. Fresh frozen tissue sections from d80 and d117 were assayed according to the manufacturer's protocol. Briefly, the fresh-frozen tissue sections were fixed using 4% paraformaldehyde in 1×PBS, dehydrated, and treated with the Protease IV kit component. The first four probes were imaged after completing the manufacturer's specified hybridization steps, counterstaining, and coverslipping. Images of tissue sections were obtained using an Nikon Ti2 with an Aura light engine (Lumencor, Beaverton, OR), and BrightLine Sedat filter set optimized for DAPI, FITC, TRITC, Cy5 & Cy7 (Semrock, Rochester, NY: LED-DA/FI/TR/Cy5/Cy7-5X5 μM-A-000) or a Yokogawa CSU-X1 spinning disk confocal microscope (Yokogawa Corporation, Sugar Land, TX) with a Celesta light engine (Lumencor), ORCA-Fusion scientific CMOS camera (Hamamatsu Corp, Bridgewater, NJ), and a HS-625 high speed emission filter wheel (Finger Lakes Instrumentation, Lima, NY). Coverslips were removed, the first four fluorophores were cleaved, and the process was repeated for probes 5-8 and then probes 9-12 (File S4). Images were analyzed using Fiji (ImageJ2 v2.3.0) and QuPath (v0.3.0) quantitative pathology and bioimage analysis freeware (Bankhead et al., 2017). Briefly, The DAPI channel images for imaging rounds two and three were aligned to the DAPI image for imaging round one using the BigDataViewer>BigWarp plugin in Fiji. Matching reference points were identified across the DAPI images and the resultant landmark tables were used in a custom .groovy script (File S5) to align the FITC, Cy3, Cy5, and Cy7 images from the three rounds of imaging. Images were uniformly background corrected and scaled as indicated in File S4. Cellular segmentation was performed in QuPath and positive signal foci and clusters were identified as subcellular detections. Parameters were set to allow for detection of foci while avoiding false positive detection events using positive and negative control images. From QuPath, the coordinates and the number of spots estimated (sum of individual puncta and estimated number of transcripts for clustered signal) for each segmented cell were processed using custom R scripts to map cell locations and expression levels. Out of the transcripts assayed by RNAScope, probe set criteria (File S3) used to identify a given cell population in RNAScope data was selected based on differential expression across the cell types identified in the sci-RNA-seq data at corresponding time points (Figure S3C). Cells matching expression criteria for a cluster's probe set were designated by cluster color and mapped spatially.

### In Vitro Differentiation

Briefly, hiPSCs (WTC-11 human induced pluripotent stem cells) (Coriell, #GM25256) were seeded on 12-well plates coated with growth factor-reduced Matrigel (Corning, #356231) and cultured in mTeSR1 stem cell medium (StemCell Technologies, #85850) until cells reach confluency with medium changes daily. On the first day of differentiation (deemed Day 0), stem cell media is replaced with ameloblast base media consisted of either EpiCult-C media (StemCell Technologies, #05630) or RPMI 1640 Medium (Thermo, #11875093) mixed with EpiLife (Thermo, #MEPI500CA) at 1:1 ratio, supplemented with 0.1× supplement S7 (Thermo, #S0175), 0.1 uM β-mercaptoethanol (BME) (Sigma, #M7522) and 400 um smoothened agonist (SAG) (Selleckchem, #S7779). At day 3 of differentiation 150 μM of bone morphogenic protein-4 (BMP4) (rndsystems, #314-BP-010) is continuously added daily till day 7. At day 8, the base media is supplemented with 1 uM of BMP-I inhibitor (LDN-193189) (Tocris, #6053), 5 uM of GSK3-Inhibitor (CHIR99021) (Selleckchem, #4423), 500 μM epidermal growth factor (EGF) (rndsystems, #236-EG) and 3.5 μM of Neurotrophin-4 (NT4) (rndsystems, #268-N4). The cultures were then harvested at day 10 at an oral epithelium stage, or extended to day 16 by adding 300 μM BMP4, and 800 nM transforming growth factor beta 1(TGFβ1) (rndsystems, #7754-BH) for the early ameloblast stage at day 16. For testing FGFR signaling requirement for the maturation process we added 50 nM purified FGFR-mb (see below) to the media at day 14 and harvested the samples at day 16 of the differentiation.

### De Novo FGFR-Miniprotein Expression

The gene encoding the designed FGFR-mb protein sequence was synthesized and cloned into modified pET-29b(+) E. coli plasmid expression vectors (GenScript, N-terminal 8-His tag followed by a TEV cleavage site). The sequence of the N-terminal tag is MSHHHHHHHHSENLYFQSGGG (SEQ ID NO. 2), which is followed immediately by the sequence of the designed protein. Plasmids were transformed into chemically competent E. coli Lemo21 cells (NEB). The protein expression was performed using Studier autoinduction medium supplemented with antibiotic, and cultures were grown overnight. Then, IPTG was added to a final concentration of 500 mM and the cells were grown overnight at 22° C. for expression. The cells were collected by spinning at 4,000 g for 10 min and then resuspended in lysis buffer (300 mM NaCl, 30 mM Tris-HCL (pH 8.0), with 0.25% CHAPS for cell assay samples) with DNase and protease inhibitor tablets. The cells were lysed with a sonicator (Qsonica Sonicators) for 4 min in total (2 min each time, 10 s on, 10 s off) with an amplitude of 80%. The soluble fraction was clarified by centrifugation at 20,000 g for 30 min. The soluble fraction was purified by immobilized metal affinity chromatography (Qiagen) followed by FPLC SEC (Superdex 75 10/300 GL, GE Healthcare). The protein samples were characterized by SDS-PAGE, and purity was greater than 95%. Protein concentrations were determined by absorbance at 280 nm measured with a NanoDrop spectrophotometer (Thermo Scientific) using predicted extinction coefficients.

### RNA Extraction and RT-qPCR Analysis

RNA was extracted using Trizol (Life Technologies) according to manufacturer's instructions. RNA samples were treated with Turbo DNase (Thermo Fisher Scientific) and quantified using Nanodrop ND-1000. Reverse transcription was performed using iScript cDNA Synthesis Kit (Bio-Rad). 10 ng of cDNA was used to perform QRT-PCR using SYBR Green (Applied Biosystems) on a 7300 real time PCR system (Applied Biosystems). The PCR conditions were set up as the following: stage 1 as 50° C. for 2 mins, stage 2 as 95° C. for 10 mis, 95° C. for 15 sec, 60° C. for 1 min (40 Cycles). β-actin was used as an endogenous control. The primer sequences used in this work are available in File S6.

### Development of Ameloblast Organoid

The day 16 differentiated iAM cells were trypsinized using TrypLE (Thermo Scientific) and re-plated in in 24-well ultra-low attachment plate (Corning, #4441) containing an ameloblast base medium with 10 μM ROCKi (Y-27632, Selleckchem, #S1049). The organoid cultures were maintained at 37° C. in 5% CO2, and the medium was changed every 3-days until further analysis.

### Co-Culture Protocol for Ameloblast and Odontoblast Organoid

The day 16 differentiated iAM cells were cultured in ultra-low attachment 12-well plate for a week in ameloblast base medium. The odontogenic organoids were made in a similar manner in a separate plate by culturing DPSCs (isolated from primary molar sample of young patient (Macrin et al., 2019)) in odontogenic differentiation medium containing DMEM (Gibco, #11995073) ascorbic acid (Sigma, #A8960), β-Glycerophosphate (Sigma, #35675), and dexamethasone (Sigma, #D2915), 10% FBS (Gibco, #10437028) and 1% Penicillin/Streptomycin (Gibco, #15140122). The two types of organoids were co-cultured in the same wells for two weeks, supplemented with a 1:1 mixture of both odontogenic and ameloblasts base media at 37° C. in 5% CO2. The co-culture was sampled later for further analysis.

### Co-Culture Protocol for Monolayer

The DPSCs were plated as monolayer mixed in 25% (v/v) of Matrigel (Corning, #356231) diluted in odontogenic media in a glass-bottomed 96-well plate (Corning, #3603). The following day, iAM cells suspended in the ameloblast base medium and 10 μM ROCKi (Y-27632, Selleckchem, #S1049) were added on top of the DPSCs monolayer and then incubated for 24 hours at 37° C. in 5% CO2. The formed organoids were supplemented with fresh media (1:1 mixture ameloblast and odontogenic media) containing Calcein solution (Sigma, #C0875) (1 uM, 1:1000) on every three consecutive days. The co-culture was sampled on the 14th day for further analysis.

### Cryosectioning and Immunostaining for the Organoids

The organoids were imbedded in OCT compound (Tissue-Tek, #4583) and slowly frozen on a metal block chilled on dry ice. Frozen organoids were cut using Cryostat (Leica CM1850) to create 10 μm slices and fixed on glass slides (Fiserbrand, #12-55015) for staining. The organoid sections were fixed in 4% paraformaldehyde (PFA) for 10-15 min at RT and later washed thrice with 1×PBS for 5 min each. Slides were then immersed in 0.5% TritonX 100 at RT for 5 minutes to facilitate permeabilization. Later blocking was done for 1 hour at RT in a humidified chamber with a blocking buffer consisting of 0.1% Triton X-100 and 5% Bovine Serum Albumin (VWR). The organoids were incubated in primary antibodies (File S6) overnight at 4° C. in a humidified chamber. After 3×5 minute washes in PBS in a coplin jar, the slides were transferred to a humidified chamber with secondary antibodies. Secondary antibodies and Phalloidin (File S6) were applied for 1 hour at RT in the same blocking agent, followed by rinsing the slides with PBS 3×5 min in coplin jar. The slides were incubated in autofluorescence quenching solution (Vector Labs, #SP-8400) for 5 min at RT under dark conditions and rinsed 1× with PBS. DAPI (Thermo Fisher) was applied for 10 minutes at room temperature in PBS. Slides were then rinsed with PBS for 10 minutes in a coplin jar. Slides were then mounted with Vectashield (Vector Labs) and stored at 4° C. for imaging.

### Wholemount Immunostaining Analysis

The organoids were collected in a 2 ml tube after two weeks and washed thoroughly with 1×PBS before fixation. The organoids were fixed in 4% paraformaldehyde (PFA) for 10-15 min at RT on a rocker. Later the fixed organoids were washed thrice with 1×PBS for 5 min each. The organoids were then immersed in 0.5% TritonX 100 at RT on a rocker for 5 minutes. Later blocking was done for 1 hour at RT on a rocker with a blocking buffer consisting of 0.1% Triton X-100 and 5% goat serum (VWR). The organoids were incubated overnight in the primary antibodies (File S6) at 4° C. on a rocker. After 5-minute washes in PBS for thrice in a coplin jar, the organoids were incubated with secondary antibodies (File S6) for an hour at RT on a rocker. The primary and the secondary antibodies were prepared in the blocking agent consisting of 0.1% Triton X-100 and 3% goat serum (VWR). Followed by washing the organoids with PBS 3×5 min on a rocker. The organoids were incubated in autofluorescence quenching solution (Vector Labs, #SP-8400) for 5 min at RT under dark conditions on a rocker and rinsed 1× with PBS. Incubate the organoids in 200 mL of PBS containing DAPI (Thermo Fisher) for 10 min. The organoids were then rinsed with PBS, mounted with Vectashield (Vector Labs, #H-1700), and stored at 4° C. for imaging.

Injection of iPSC-Derived Ameloblast-Like Cells into Mouse Muscles

hiPSCs (WTC11) were allowed to undergo differentiation for the pre-ameloblast stage at day 16 using the following basal supplements mentioned above cultured in Matrigel. 1×10⁶ iAM cells were resuspended in Matrigel supplemented with a cocktail of prosurvival factors (Laflamme et al., 2007) and injected into the femoral muscle of SCID-Beige mice (Charles River, Wilmington, MA). Mice were kept under BioSafety containment Level 2. Mice were sacrificed and femoral muscles were harvested after 2 months and were dissected at the site of injection (left leg muscle) to perform further analysis. Experiment was performed in compliance with ethical regulations, IACUC protocol #4152-01. After dissection, left leg muscles were embedded in embedding cryo-mold (Polysciences, #18986-1) with minimum amount of Tissue-Tek O.C.T. compound (Sakura, catalog number: 4583) to cover the muscle region. The embedded tissue was then snap-frozen by placing on a cold-resistant beaker of 2-methylbutane solution (EMD. #MX0760-1) into a slurry of liquid nitrogen for 5-mins, which allows fast cooling to −80° C. The snap-frozen samples are then placed in a −80° C. freezer for storage. The cryostat and blade are both pre-chilled to −20° C. before cryo-sectioning. 10 μm-thick sections were made on pre-chilled Superfrost Plus microscope slides (Fisherbrand, #12-550-15) and then store in a −80° C.

### Calcification Assays: Von Kossa and Alizarin Red Staining

Identification of mineralization was performed on tissue sections stained with Von Kossa and Alizarin Red S. Frozen leg muscle sections (Om) were fixed with 4% paraformaldehyde (EMS, #15710) in H₂O at room temperature for 12 min. Rinse the section with deionized distilled water thrice for 5 min each. Sections were incubated in with 5% silver nitrate solution (SIGMA-ALDRICH #209139) placed under ultraviolet light for 1 hour. The section was rinsed with several changes of deionized distilled water for 5 min each and later incubated in 5% Sodium Thiosulfate solution (SIGMA-ALDRICH #217263) for 5 minute to remove un-reacted silver. Similarly, sections were stained with 2% Alizarin red S solution (pH4.2) (Sigma, #A5533) for 1 hour in the dark. The slides were thoroughly with deionized distilled water for 5 min each followed by counterstaining the sections with nuclear fast red stain (EMS, #26078-05) for 5 minutes. Rinsed in deionized distilled water briefly for 5 mins each the slides were successfully transferred into coplin jars to perform dehydration step through graded alcohol and clear the slides in CitriSolv solution (Decon, #1601). Slides were then mounted with Vectashield (Vector Labs, #H-1400-10) and stored at room temperature for imaging.

### Cryosectioning of Fetal Samples

Jaw tissues were fixed with 4% PFA overnight at 4° C. followed by 30% sucrose (Sigma, #RDD023) treatment until the tissue sank to the bottom of the tube. The tissue is then imbedded in OCT compound (Tissue-Tek, #4583) and slowly frozen on a metal block chilled on dry ice. Frozen samples were cut using Cryostat (Leica CM1850) to create 10 μm slices of tissue and fixed on glass slides (Fiserbrand, #12-55015) for staining.

### Immunofluorescence Staining and Confocal Imaging

Toothgerms embedded in O.C.T. were cryosectioned to 10-micron thick sections. The slides were stored at −80° C. after cryosectioning and warmed at room temperature prior to staining. Tissues were fixed in 4% paraformaldehyde (PFA) then immersed in 1×PBS for 3×5 minute washes. Antigen retrieval was performed using 10× Citrate Buffer (Sigma-Aldrich) in a capped coplin jar microwaved for ~45 seconds followed by 15-minutes incubation in microwave. Slides were then allowed to be washed in PBS at room temperature for 7 minutes. Slides were blocked for 90 minutes at room temperature in a humidified chamber with a blocking buffer consisting of 0.1% Triton X-100 and 5% Bovine Serum Albumin (VWR). All the antibodies used in this study and their concentrations are listed in File S6. The primary antibodies were incubated overnight at 4° C. in a humidified chamber. After 3×5 minute washes in PBS in a coplin jar, the slides were transferred to a humidified chamber with secondary antibodies. Secondary antibodies were applied for 75 minutes at room temperature in the same blocking agent. Slides were then rinsed with PBS 4×10 minute washes in a coplin jar. DAPI (Thermo Fisher) was applied for 10 minutes at room temperature in PBS. Slides were then rinsed with PBS for 10 minutes in a coplin jar. Slides were then mounted with Vectashield (Vector Labs) and stored at 4° C. for imaging. Confocal Imaging was done on a Leica TCS-SPE Confocal microscope using a 40× objective and Leica Software. Images were processed with Fiji software distribution of ImageJ v2.3.0 (Schindelin et al., 2012; Schindelin et al., 2015). NIS-Elements (RRID:SCR_014329) was used for 3D reconstruction.

### Data Availability

The data generated in this study can be downloaded in raw and processed forms from the NCBI Gene Expression Omnibus under accession number (GSE184749). Upon publication, raw RNAScope data will be made publicly available on drvad.org (Dryad research data repository).

### Code Availability

The custom R codes used to generate some of the results in this disclosure are available in github.com/Ruohola-Baker-lab/Tooth_sciRNAseq.

### Example 2

### Designed Protein Mini Binders can be Used to Study and Enhance iOE and iAM Differentiation

To test the effect of protein-designed receptor mini binders on the induced oral epithelium (iOE) stage of the differentiation (between day 8 and day 10) the differentiation media was supplemented with a 100 nM concentration of the binder (EGFR mb) (Cao et al. 2022; Natasha I Edman 2022). As a readout of the efficiency of the differentiation in the presence of these binders, the expression level was evaluated of three oral epithelium markers that are downregulated in iAM (PITX2, KRT14, and TBX1) (detected by RT-qPCR in FIG. 17).

EGFR_mb binds to the epidermal growth factor receptor (EGFR) and has been shown to act as an inhibitor that competes with the natural ligand EGF. In iOE differentiation, in which EGF ligands are normally used, it was found that adding EGFR_mb induces a significant reduction in all three OE markers tested. This suggests that the EGF pathway is essential for iOE differentiation.

### REFERENCES

Cao, L., B. Coventry, I. Goreshnik, B. Huang, W. Sheffler, J. S. Park, K. M. Jude, I. Marković, R. U. Kadam, K. H. G. Verschueren, K. Verstraete, S. T. R. Walsh, N. Bennett, A. Phal, A. Yang, L. Kozodoy, M. DeWitt, L. Picton, L. Miller, E. M. Strauch, N. D. DeBouver, A. Pires, A. K. Bera, S. Halabiya, B. Hammerson, W. Yang, S. Bernard, L. Stewart, I. A. Wilson, H. Ruohola-Baker, J. Schlessinger, S. Lee, S. N. Savvides, K. C. Garcia, and D. Baker. 2022. ‘Design of protein-binding proteins from the target structure alone’, Nature, 605: 551-60.

1. A method of preparing an ameloblast culture, the method comprising, in order,
a) contacting, in culture, an induced pluripotent stem cell (iPSC) with an activator of the Hedgehog pathway;
b) adding Bone Morphogenetic Protein 4 (BMP4) to the culture of (a);
c) adding an inhibitor of BMP type I receptors, a Wnt activator, Epidermal Growth Factor (EGF) and Neurotrophin-4 (NT4) to the culture of (b) and incubating to form oral epithelium cells; and
d) adding BMP4 and transforming growth factor β1 (TGF-μ1) to the culture of (c) and incubating to form ameloblasts.

2. The method of claim 1, wherein the ameloblasts express ameloblastin.

3. The method of claim 1, wherein the cells are human.

4. The method of claim 1, wherein the iPSCs are at least one of the following:
seeded on tissue culture plates coated with an extracellular matrix composition,
grown to confluence prior to step (a), and
cultured to confluence in mTeSR1 stem cell medium.

5. The method of claim 4, wherein the extracellular matrix composition comprises a natural or a synthetic extracellular matrix composition.

6. (canceled)

7. (canceled)

8. The method of claim 1, wherein at least one of:
the Hedgehog activator is smoothened agonist (SAG),
the inhibitor of BMP type I receptors is LDN-193189,
the Wnt activator is a GSK-3 inhibitor, and
the Wnt activator is a GSK-3 inhibitor that is CHIR99021.

9. The method of claim 4, wherein confluent iPS cells are switched to medium comprising the Hedgehog activator at day zero of differentiation.

10. The method of claim 8, wherein SAG is added at 200 nM to 1 μM,
wherein SAG is added at 400 nM,
wherein the LDN-193189 is added at 100 nM to 5 μM,
wherein LDN-193189 is added at 1 μM,
wherein CHIR99021 is added at 0.5 μM to 50 μM,
wherein the CHIR99021 is added at 5 μM,
wherein EGF is added at 50 pM to 5 nM,
wherein EGF is added at 500 pM,
wherein NT4 is added at 350 nM to 35 μM,
wherein NT4 is added at 3.5 μM,
wherein the TGF-β1 is added at 80 nM to 8 μM, and/or
wherein the TGF-β1 is added at 800 nM.

11. (canceled)

12. The method of claim 1, wherein addition of BMP4 step (b) is performed at day 3 of differentiation,
wherein addition of BMP4 step (b) adds BMP4 at 100 pM to 750 pM,
wherein addition of BMP4 step (b) adds BMP4 at 150 pM,
wherein cells are incubated with BMP4 of step (b) from day 3 until day 7 of differentiation,
wherein the BMP-4 added at step (d) is added at 30 pM to 3 nM, and/or
wherein the BMP-4 added at step (d) is added at 300 pM.

13.-15. (canceled)

16. The method of claim 1, wherein step (c) addition of an inhibitor of BMP type I receptors, a Wnt activator, EGF and NT4 is performed at day 8 of differentiation.

17.-27. (canceled)

28. The method of claim 1, wherein step (d) addition of BMP4 and TGF-β1 is performed at day 10 of differentiation, and/or
wherein step (d) addition of BMP4 and TGF-β1 is performed when expression of one or more of PITX2, TBX1 and TP63 is detected in the differentiating culture.

29.-33. (canceled)

34. The method of claim 1, wherein the incubating of step (d) is to day 16 or more of differentiation.

35. A cultured organoid comprising in an in vitro-differentiated ameloblast.

36. The cultured organoid of claim 35, wherein the ameloblast is differentiated from an iPS cell.

37. The cultured organoid of claim 35, wherein the ameloblast is human.

38. The cultured organoid of claim 35, wherein ameloblastin is secreted into a lumen in the organoid.

39.-42. (canceled)

43. A dental repair composition comprising ameloblastin or enamel produced by an in vitro-differentiated cell.

44. (canceled)

45. The dental repair composition of claim 43, which further comprises hydroxyapatite or calcium phosphate, and/or
further comprises one or more of amelogenin and enamelin.

46.-48. (canceled)

49. A method of repairing a tooth, the method comprising contacting a tooth with a dental repair composition of claim 43.

50. A method of treating amelogenesis imperfecta, the method comprising administering a composition comprising an in vitro-differentiated ameloblast to a subject in need thereof.

51.-62. (canceled)
